(19) United States
(12) Patent Application Publication
(10) Pub. No.: US 2023/0032087 A1
(43) Pub. Date: Feb. 2, 2023

(54) ANTI-SERUM ALBUMIN ANTIBODIES

(71) Applicant: Cullinan Oncology, Inc., Cambridge, MA (US)

(72) Inventors: Patrick A. Baeuerle, Cambridge, MA (US); Jennifer Michaelson, Cambridge, MA (US); Bochong Li, Boston, MA (US); Naveen Mehta, Cambridge, MA (US); Bianka Prinz, Lebanon, NH (US); Bradley M. Lunde, Lebanon, NH (US); Nga Rewa Houston, Lebanon, NH (US)

(21) Appl. No.: 17/785,011

(22) PCT Filed: Dec. 11, 2020
(86) PCT No.: PCT/US2020/064672

§ 371 Date: Jun. 13, 2022

Related U.S. Application Data
(60) Provisional application No. 62/946,932, filed on Dec. 11, 2019.

(51) Int. Cl. C07K 16/28; C07K 16/46

(52) U.S. Cl. CPC C07K 16/2809; C07K 16/468; A61K 2039/505

(57) ABSTRACT

The invention relates to anti-serum albumin antibodies and multi-specific binding proteins comprising the same. The invention also relates to pharmaceutical compositions comprising the antibodies or multi-specific binding proteins, expression vectors and host cells for making the antibodies or multi-specific binding proteins, and methods of use of the antibodies or multi-specific binding proteins in treatment of diseases or disorders.

### CROSS-REFERENCE TO RELATED APPLICATIONS

This application claims the benefit of and priority to U.S. Provisional Patent Application No. 62/946,932, filed on Dec. 11, 2019, the disclosure of which is hereby incorporated by reference in its entirety for all purposes.

### FIELD OF THE INVENTION

The invention relates to anti-serum albumin antibodies and multi-specific binding proteins comprising the same. The invention also relates to pharmaceutical compositions comprising these antibodies or multi-specific binding proteins, expression vectors and host cells for making these antibodies or multi-specific binding proteins, and methods of use of these antibodies or multi-specific binding proteins in treatment of diseases or disorders.

### BACKGROUND

Serum albumin is the most abundant protein in serum. It has high stability, solubility, and a long circulatory half-life. Polypeptides that bind serum albumin, such as antibodies, have been developed to increase the circulatory half-life of therapeutic proteins. Although significant developments have been made, there remains a need for new and useful anti-serum albumin antibodies and multi-specific binding proteins having improved pharmacokinetic properties.

### SUMMARY OF THE INVENTION

The present invention is based, in part, upon the development of new antibodies that bind serum albumin. Also provided are multi-specific binding proteins comprising a first domain that binds a first target protein expressed on a target cell, such as CD19 (e.g., human CD19), and/or a second domain that binds a second target protein expressed on an immune effector cell, such as CD3 (e.g., human CD3), and a third domain that binds serum albumin (e.g., human serum albumin), wherein the third domain are derived from these new antibodies. These domains are linked in certain manners for favorable therapeutic efficacy and in vivo half-life. The multi-specific binding proteins are useful for treating diseases and disorders associated with aberrant cells expressing the first target protein, such as certain B-cell hematologic malignancies.

Accordingly, in one aspect, the present invention provides an antigen-binding site that binds human serum albumin, comprising a VH comprising complementarity determining regions HCDR1, HCDR2, and HCDR3, wherein the HCDR1, HCDR2, and HCDR3 comprise the amino acid sequences of SEQ ID NOs: 184, 409, and 411, respectively, but not SEQ ID NOs: 129, 133, and 135, respectively.

In certain embodiments, the HCDR1, HCDR2, and HCDR3 comprise the amino acid sequences of SEQ ID NOs: 184, 185, and 187, respectively, but not SEQ ID NOs: 129, 133, and 135, respectively. In certain embodiments, the HCDR1, HCDR2, and HCDR3 comprise the amino acid sequences of SEQ ID NOs: 189, 190, and 192, respectively, but not SEQ ID NOs: 129, 133, and 135, respectively. In certain embodiments, the HCDR1, HCDR2, and HCDR3 comprise the amino acid sequences of SEQ ID NOs: 189, 193, and 195, respectively, but not SEQ ID NOs: 129, 133, and 135, respectively. In certain embodiments, the HCDR1, HCDR2, and HCDR3 comprise the amino acid sequences of SEQ ID NOs: 123, 124, and 126, respectively. In certain embodiments, the VH comprises an amino acid sequence at least 85%, at least 90%, at least 95%, at least 99%, or 100% identical to SEQ ID NO: 121. In certain embodiments, the antigen-binding site binds human serum albumin with a K_{D} lower than or equal to 10 nM. In certain embodiments, the antigen-binding site binds protein A with a K_{D} lower than or equal to 2 nM. The antigen-binding site of any one of claims 1-9, wherein the antigen-binding site has a melting temperature greater than or equal to 60° C.

In another aspect, the instant disclosure provides a multi-specific binding protein comprising: (a) a first antigen-binding site that binds a first target protein expressed on a target cell (e.g., human CD19); (b) a second antigen-binding site that binds a second target protein expressed on an immune effector cell (e.g., human CD3); and (c) a third antigen-binding site that binds human serum albumin, wherein the third antigen-binding site is an antigen-binding site that binds human serum albumin disclosed herein.

In certain embodiments, the multi-specific binding protein comprises a single polypeptide chain. In certain embodiments, the third antigen-binding site is not positioned between the first antigen-binding site and the second antigen-binding site in the polypeptide chain.

In certain embodiments, the third antigen-binding site is positioned N-terminal to both the first antigen-binding site and the second antigen-binding site in the polypeptide chain. In certain embodiments, the third antigen-binding site is positioned N-terminal to the first antigen-binding site, and the first antigen-binding site is positioned N-terminal to the second antigen-binding site in the polypeptide chain. In certain embodiments, the third antigen-binding site is positioned N-terminal to the second antigen-binding site, and the second antigen-binding site is positioned N-terminal to the first antigen-binding site in the polypeptide chain.

In certain embodiments, the third antigen-binding site is positioned C-terminal to both the first antigen-binding site and the second antigen-binding site in the polypeptide chain. In certain embodiments, the first antigen-binding site is positioned N-terminal to the second antigen-binding site, and the second antigen-binding site is positioned N-terminal to the third antigen-binding site in the polypeptide chain. In certain embodiments, the second antigen-binding site is positioned N-terminal to the first antigen-binding site, and the first antigen-binding site is positioned N-terminal of the third antigen-binding site in the polypeptide chain.

In certain embodiments, the first antigen-binding site is positioned N-terminal to the third antigen-binding site, and the third antigen-binding site is positioned N-terminal to the second antigen-binding site in the polypeptide chain. In other embodiments, the second antigen-binding site is positioned N-terminal to the third antigen-binding site, and the third antigen-binding site is positioned N-terminal binding protein the first antigen-binding site in the polypeptide chain.

In certain embodiments, the first antigen-binding site comprises a single-chain variable fragment (scFv). In certain embodiments, the third antigen-binding site comprises a single domain antibody (sdAb). In certain embodiments, the second antigen-binding site comprises an scFv.

In certain embodiments, the second antigen-binding site binds human CD3ε. In certain embodiments, the second antigen-binding site binds human CD3ε with a K_{D} in the range of 1-100 nM.

In certain embodiments, the second antigen-binding site comprises a VH comprising complementarity determining regions HCDR1, HCDR2, and HCDR3, and a VL comprising complementarity determining regions LCDR1, LCDR2, and LCDR3, wherein the HCDR1, HCDR2, HCDR3, LCDR1, LCDR2, and LCDR3 comprise the amino acid sequences set forth in SEQ ID NOs: 415, 416, 418, 419, 420, and 421, respectively. In certain embodiments, the VH comprises an amino acid sequence at least 85%, at least 90%, at least 95%, at least 99%, or 100% identical to SEQ ID NO: 412, and the VL comprises an amino acid sequence at least 85%, at least 90%, at least 95%, at least 99%, or 100% identical to SEQ ID NO: 413. In certain embodiments, the antigen-binding site comprises the amino acid sequence of SEQ ID NO: 422 or 423.

In certain embodiments, the second antigen-binding site comprises a VH comprising complementarity determining regions HCDR1, HCDR2, and HCDR3, and a VL comprising complementarity determining regions LCDR1, LCDR2, and LCDR3, wherein the HCDR1, HCDR2, HCDR3, LCDR1, LCDR2, and LCDR3 comprise the amino acid sequences set forth in SEQ ID NOs: 415, 416, 426, 419, 420, and 421, respectively. In certain embodiments, the VH comprises an amino acid sequence at least 85%, at least 90%, at least 95%, at least 99%, or 100% identical to SEQ ID NO: 424, and the VL comprises an amino acid sequence at least 85%, at least 90%, at least 95%, at least 99%, or 100% identical to SEQ ID NO: 413. In certain embodiments, the antigen-binding site comprises the amino acid sequence of SEQ ID NO: 427 or 428.

In certain embodiments, the second antigen-binding site comprises a VH comprising complementarity determining regions HCDR1, HCDR2, and HCDR3, and a VL comprising complementarity determining regions LCDR1, LCDR2, and LCDR3, wherein the HCDR1, HCDR2, HCDR3, LCDR1, LCDR2, and LCDR3 comprise the amino acid sequences set forth in SEQ ID NOs: 415, 431, 418, 419, 420, and 432, respectively. In certain embodiments, the VH comprises an amino acid sequence at least 85%, at least 90%, at least 95%, at least 99%, or 100% identical to SEQ ID NO: 429, and the VL comprises an amino acid sequence at least 85%, at least 90%, at least 95%, at least 99%, or 100% identical to SEQ ID NO: 430. In certain embodiments, the antigen-binding site comprises the amino acid sequence of SEQ ID NO: 433 or 434.

In certain embodiments, at least two adjacent antigen-binding sites are connected by a peptide linker. In certain embodiments, each of the adjacent antigen-binding sites are connected by a peptide linker. In certain embodiments, the peptide linker comprises the amino acid sequence of SEQ ID NO: 298, 299, or 302. In certain embodiments, the peptide linker consists of the amino acid sequence of SEQ ID NO: 298, 299, or 302.

In certain embodiments, the multi-specific binding protein does not comprise an antibody Fc region. In certain embodiments, the molecular weight of the multi-specific binding protein is at least 65 kD. In certain embodiments, the serum half-life of the multi-specific binding protein is at least 24, 36, 48, or 60 hours.

The instant disclosure also provides an antibody comprising an antigen-binding site that binds human serum albumin disclosed herein.

In another aspect, the instant disclosure provides a pharmaceutical composition comprising: (a) a multi-specific binding protein or an antibody disclosed herein; and (b) a pharmaceutically acceptable carrier.

The instant disclosure also provides an isolated polynucleotide encoding a multi-specific binding protein or an antibody disclosed herein. In addition, the instant disclosure provides a vector comprising the polynucleotide disclosed herein, and a recombinant host cell comprising the polynucleotide or vector disclosed herein.

The instant disclosure also provides a method of producing a multi-specific binding protein or an antibody, the method comprising culturing a host cell disclosed herein under suitable conditions that allow expression of the multi-specific binding protein or the antibody. In certain embodiments, the method further comprises isolating the multi-specific binding protein or the antibody. In certain embodiments, the method further comprises formulating the isolated multi-specific binding protein or antibody with a pharmaceutically acceptable carrier.

In addition, the instant disclosure provides a method of stimulating an immune response against a target cell, the method comprising exposing the cell and a T lymphocyte to a multi-specific binding protein, an antibody, or a pharmaceutical composition disclosed herein.

The instant disclosure also provides a method of treating a hematologic cancer in a subject in need thereof, the method comprising administering to the subject an effective amount of a multi-specific binding protein, an antibody, or a pharmaceutical composition disclosed herein. In certain embodiments, the hematologic cancer is a B-cell hematologic malignancy.

In addition, the instant disclosure provides a complex comprising a T cell expressing CD3, a B cell expressing CD19, and a multi-specific binding protein disclosed herein, wherein the multi-specific binding protein simultaneously bind both the T cell and the B cell. In certain embodiments, the complex further comprises serum albumin.

### BRIEF DESCRIPTION OF THE DRAWINGS

FIG. 1 is a schematic representation of six domain arrangements of single-chain multi-specific binding proteins. The CD19 binding domain in the form of an scFv, the CD3 binding domain in the form of an scFv, and the HSA binding domain in the form of an sdAb are linked in different orientations. The top of each construct represents the N-terminus and the bottom of each construct represents the C-terminus of a given polypeptide chain.

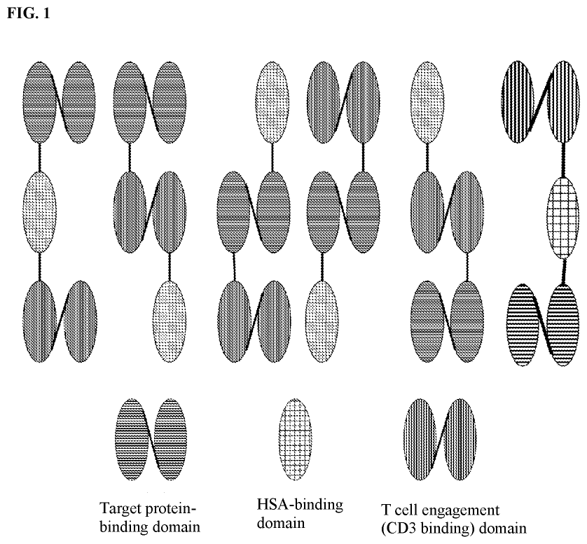

### DETAILED DESCRIPTION

The present invention is based, in part, upon the development of new antibodies that bind serum albumin. Also provided are multi-specific binding proteins comprising a first domain that binds a first target protein expressed on a target cell, such as CD19 (e.g., human CD19), and/or a second domain that binds a second target protein expressed on an immune effector cell, such as CD3 (e.g., human CD3), and a third domain that binds serum albumin (e.g., human serum albumin), wherein the third domain is derived from these new antibodies. These domains are linked in certain manners for favorable therapeutic efficacy and in vivo half-life. The multi-specific binding proteins are useful for treating diseases and disorders associated with aberrant cells expressing the first target protein, such as certain B-cell hematologic malignancies.

To facilitate an understanding of the present invention, a number of terms and phrases are defined below.

The term “multi-specific binding protein” refers to a protein or protein conjugate capable of binding two or more different targets (e.g., two or more different antigens or two or more different epitopes of the same antigen). For example, the multi-specific binding protein can bind two or more different targets through two or more different binding domains. The structure and/or function of the multi-specific binding protein can be based on the structure and/or function of an antibody, e.g., a full-length or whole immunoglobulin molecule, an antibody heavy chain variable domain (VH) and/or light chain variable domain (VL), and/or a single chain antibody. In one example, each one of the binding domains of a multi-specific binding protein according to the invention comprises the minimum structural requirements of an antibody which allow for the target binding. This minimum requirement may be, e.g., defined by the presence of at least the three heavy chain CDRs (i.e. CDR1, CDR2 and CDR3 of the VH domain) and/or the three light chain CDRs (i.e. CDR1, CDR2 and CDR3 of the VL domain). An alternative approach to defining the minimal structural requirements of an antibody is defining the epitope of a specific target to which the antibody binds, or by referring to a known antibody with which the antibody competes to bind to the same epitope that the known antibody binds. The antibodies on which the constructs according to the invention are based include for example monoclonal, recombinant, chimeric, deimmunized, humanized and human antibodies.

Any one of the binding domains of a multi-specific binding protein according to the invention may comprise the above referred groups of CDRs. Those CDRs may be comprised in the framework of a VH and/or VL. Fd fragments, for example, have two VH domains and often retain some antigen-binding function of the intact antigen-binding domain. Additional examples for formats of antibody fragments, antibody variants or binding domains include: (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody; (5) a dAb fragment (Ward et al., (1989) Nature 341:544-546), which has a VH domain; (6) an isolated complementarity determining region (CDR); and (7) a single chain Fv (scFv), which may be derived, for example, from an scFv-library. Exemplary formats of multi-specific binding proteins according to the invention are described in, e.g., WO2000006605A2, WO2005040220A1, WO2008119567A2, WO2010037838A2, WO2013026837A1, WO 2013026833A1, US 20140308285A1, US20140302037A1, WO2014144722A2, WO2014151910A1, and WO2015048272A1.

Multi-specific binding proteins according to the invention may also comprise modified fragments of antibodies, also called antibody variants, such as di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, or single-domain antibodies such as nanobodies or single variable domain antibodies comprising a single variable domain, which might be VH (also called VHH in the context of an sdAb) or VL, that specifically bind an antigen or epitope independently of other V regions or domains.

As used herein, the terms “single-chain Fv,” “single-chain antibody,” and “scFv” refer to a single-polypeptide-chain antibody fragment that comprise the variable regions from both the heavy and light chains, but lack the constant regions. Generally, a single-chain antibody further comprises a peptide linker connecting the VH and VL domains which enables it to form the desired structure to bind to antigen. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, humanized and/or synthetic.

Furthermore, the “multi-specific binding protein” described herein can be a monovalent, bivalent or polyvalent/multivalent construct. Moreover, the “multi-specific binding protein” described herein can include a molecule consisting of only one polypeptide chain, or a molecules consisting of more than one polypeptide chain, wherein the chains can be either identical (homodimers, homotrimers or homo oligomers) or different (heterodimer, heterotrimer or heterooligomer). Examples for the above identified antibodies and the variants or derivatives thereof are described, for example, in Harlow and Lane, Antibodies a laboratory manual, CSHL Press (1988); Using Antibodies: a laboratory manual, CSHL Press (1999); Kontermann and Dibel, Antibody Engineering, Springer, 2nd ed. 2010; and Little, Recombinant Antibodies for Immunotherapy, Cambridge University Press 2009.

The domains of the multi-specific binding protein of the present invention may be connected through one or more peptide bonds and/or peptide linkers. The term “peptide linker” comprises in accordance with the present invention an amino acid sequence linking two domains. The peptide linkers can also be used to fuse the third domain to the other domains of the multi-specific binding protein of the invention. An essential technical feature of such peptide linker is that it does not comprise any polymerization activity. Among the suitable peptide linkers are those described in U.S. Pat. Nos. 4,751,180 and 4,935,233 or WO198809344A1.

The multi-specific binding proteins of the present invention may be in vitro generated multi-specific binding proteins. The term “in vitro generated multi-specific binding protein” refers to a multi-specific binding protein according to the above definition where all or part of the variable region (e.g., at least one CDR) is generated by non-immune cell selection, e.g., an in vitro phage display, protein chip or any other method in which candidate sequences can be tested for their ability to bind to an antigen. The multi-specific binding proteins of the present invention may also be generated by genomic rearrangement in an immune cell in an animal. A “recombinant antibody” is an antibody made through the use of recombinant DNA technology or genetic engineering.

The multi-specific binding protein of the invention may be monoclonal. The term “monoclonal,” as used herein, means that the proteins obtained from a population are substantially homogeneous, i.e., the individual proteins in the population are identical except for naturally occurring mutations and/or post-translation modifications (e.g., isomerizations, amidations) that may be present. In the context of antibodies, monoclonal antibodies are highly specific, being directed against a single antigenic side or determinant on the antigen, in contrast to conventional (polyclonal) antibody preparations which typically include different antibodies directed against different determinants (or epitopes). The modifier “monoclonal” indicates the character of the antibody as being obtained from a substantially homogeneous population of antibodies, and is not to be construed as requiring production of the antibody by any particular method.

The multi-specific binding protein of the invention or one or more antigen-binding site thereof may be affinity matured. In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. Two or three rounds of mutation and selection using display methods such as phage display can result in antibody fragments with affinities in the low nanomolar range.

An amino acid substitution variation can be introduced into the multi-specific binding proteins by substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e.g., 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domains. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The multi-specific binding proteins of the present invention specifically can comprise “chimeric” antibodies (immunoglobulins) or fragments thereof in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, so long as they exhibit the desired biological activity (U.S. Pat. No. 4,816,567; Morrison et al. (1984) Proc. Natl. Acad. Sci. U.S.A., 81: 6851-55). Chimeric antibodies of interest herein include “primatized” antibodies comprising variable domain antigen-binding sequences derived from a non-human primate (e.g., Old World Monkey, Ape etc.) or human constant region sequences. A variety of approaches for making chimeric antibodies have been described. See e.g., Morrison et al. (1985) Proc. Natl. Acad. Sci. U.S.A., 81:6851; Takeda et al. (1985) Nature, 314: 452; U.S. Pat. Nos. 4,816,567; 4,816,397; European Patent No. EP0171496; European Patent Application Publication No. EP0173494; and U.K. Patent No. GB2177096.

The term “binding domain” or “domain that binds (an antigen)” characterizes in connection with the present invention a domain which (specifically) binds to or interacts with a given target epitope or a given target side on the target molecules (antigens), e.g. CD19, serum albumin, and CD3, respectively. The structure and function of the first binding domain, the second binding domain, and/or the third binding domain can be based on the structure and/or function of an antibody, e.g. of a full-length or whole immunoglobulin molecule. A binding domain can be drawn from the VH and/or VL or VHH domain of an antibody or fragment thereof. For example, a binding domain can include three light chain CDRs (i.e., CDR1, CDR2 and CDR3 of the VL domain) and/or three heavy chain CDRs (i.e., CDR1, CDR2 and CDR3 of the VH domain). A binding domain can also include VHH CDRs (i.e., CDR1, CDR2 and CDR3 of the VHH region).

The terms “variable domain” and “variable region” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface.

In the present invention, any one of the binding domains of the multi-specific binding protein may comprise a single domain antibody (sdAb). A single domain antibody comprises a single, monomeric antibody variable domain which is able to bind selectively to a specific antigen, independently of other variable regions or domains. The first single domain antibodies were engineered from heavy chain antibodies found in camelids, and these are called WEI fragments. Cartilaginous fishes also have heavy chain antibodies (IgNAR) from which single domain antibodies called V_{NAR} fragments can be obtained. An alternative approach is to split the dimeric variable domains from common immunoglobulins e.g., from humans or rodents into monomers, hence obtaining VH or VL as a single domain antibody. Although most research into single domain antibodies is currently based on heavy chain variable domains, nanobodies derived from light chains have also been shown to bind specifically to target epitopes. Examples of single domain antibodies include nanobodies and single variable domain antibodies.

As used herein, the term “antigen-binding site” refers to the part of an immunoglobulin molecule or a derivative or variant thereof that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

As used herein, the term “antibody” refers to a protein or a protein conjugate that comprises an antigen-binding site. An antibody can be monospecific or multi-specific (e.g., bispecific).

The terms “a” and “an” as used herein mean “one or more” and include the plural unless the context is inappropriate.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. (1975).

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### 1. Anti-Serum Albumin Antibodies

In one aspect, the present disclosure provides an antigen-binding site that binds serum albumin (e.g., human serum albumin (HSA)) derived from the single domain antibodies listed in Table 1. The present disclosure also provides an antibody comprising the antigen-binding site. The CDR sequences are identified under the Kabat numbering scheme unless indicated by an asterisk (*).

TABLE 1
Sequences of Exemplary Antibodies That Bind Serum Albumin

| Antibody | VH and HCDRs |
|---|---|
| CNG-HSA- | KVQLVESGGGLVQPGGSLRLSCAASGFTFSSFGMTWVRQAPGKGLEWVSSISG |
| 101 | SGSDTLYADSVRGRFTISRDNSKNTLYLQMNSLRAEDTAVYYCTIGGSLSPSSQ |
| | GTLVTVSS (SEQ ID NO: 121) |
| | HCDR1*: FTFSSFGMT (SEQ ID NO: 122) |
| | HCDR1: SFGMT (SEQ ID NO: 123) |
| | HCDR2: SISGSGSDTLYADSVRG (SEQ ID NO: 124) |
| | HCDR3*: TIGGSLSP (SEQ ID NO: 125) |
| | HCDR3: GGSLSP (SEQ ID NO: 126) |
| CNG-HSA- | KVQLLESGGGLVQPGGSLRLSCAASGFTFSSFGMSWVRQAPGKGLEWVSSISG |
| 102 | SGSDTLYAD S VRGRFTISRDNSKNTLYLQMNSLRPEDTAVYYCTIGGSLSPSSQ |
| | GTLVTVSS (SEQ ID NO: 127) |
| | HCDR1*: FTFSSFGMS (SEQ ID NO: 128) |
| | HCDR1: SFGMS (SEQ ID NO: 129) |
| | HCDR2: SISGSGSDTLYADSVRG (SEQ ID NO: 124) |
| | HCDR3*: TIGGSLSP (SEQ ID NO: 125) |
| | HCDR3: GGSLSP (SEQ ID NO: 126) |
| CNG-HSA- | KVQLVESGGGLVQPGGSLRLSCAASGFTFSSFGMTWVRQAPGKGLEWVSSISG |
| 103 | SGSDTLYADSVRGRFTISRDNSKNTLYLQMNSLRPEDTAVYYCTIGGSLSPSSQ |
| | GTLVTVSS (SEQ ID NO: 130) |
| | HCDR1*: FTFSSFGMT (SEQ ID NO: 122) |
| | HCDR1: SFGMT (SEQ ID NO: 123) |
| | HCDR2: SISGSGSDTLYADSVRG (SEQ ID NO: 124) |
| | HCDR3*: TIGGSLSP (SEQ ID NO: 125) |
| | HCDR3: GGSLSP (SEQ ID NO: 126) |
| CNG-HSA- | KVQLLESGGGLVQPGGSLRLSCAASGFTFHSFGMSWVRQAPGKGLEWVSSISG |
| 104 | SGSDTLYADSVKGRFTISRDNSKNTLYLQMNSLRPEDTAVYYCTIGGSLSRSSQ |
| | GTLVTVSS (SEQ ID NO: 131) |
| | HCDR1*: FTFHSFGMS (SEQ ID NO: 132) |
| | HCDR1: SFGMS (SEQ ID NO: 129) |
| | HCDR2: SISGSGSDTLYADSVKG (SEQ ID NO: 133) |
| | HCDR3*: TIGGSLSR (SEQ ID NO: 134) |
| | HCDR3: GGSLSR (SEQ ID NO: 135) |
| CNG-HSA- | EVQLLESGGGLVQPGGSLRLSCAASGFVFSSFGMSWVRQAPGKGLEWVSSISG |
| 105 | SGSDTLYADSVKGRFTISRDNSKNTLYLQMNSLRPEDTAVYYCTIGGSLSRSSQ |
| | GTLVTVSS (SEQ ID NO: 136) |
| | HCDR1*: FVFSSFGMS (SEQ ID NO: 137) |
| | HCDR1: SFGMS (SEQ ID NO: 129) |
| | HCDR2: SISGSGSDTLYADSVKG (SEQ ID NO: 133) |
| | HCDR3*: TIGGSLSR (SEQ ID NO: 134) |
| | HCDR3: GGSLSR (SEQ ID NO: 135) |
| CNG-HSA- | KVQLLESGGGLVQPGGSLRLSCAASGFTFSSFGMSWVRQAPGKGLEWVSSISG |
| 106 | SGSDTLYADSVRGRFTISRDNSKNTLYLQMDSLRAEDTAVYYCTIGGSRSISSQ |
| | GTLVTVSS (SEQ ID NO: 138) |
| | HCDR1*: FTFSSFGMS (SEQ ID NO: 128) |
| | HCDR1: SFGMS (SEQ ID NO: 129) |
| | HCDR2: SISGSGSDTLYADSVRG (SEQ ID NO: 124) |
| | HCDR3*: TIGGSRSI (SEQ ID NO: 139) |
| | HCDR3: GGSRSI (SEQ ID NO: 140) |
| CNG-HSA- | KVQLLESGGGLVQPGGSLRLSCAASGFTFSSFGMSWVRQAPGKGLEWVSSISG |
| 107 | SGSDTLYADSVRGRFTISRDNSKNTLYLQMNSLRAEDTAVYYCSIGGSLIRSSQ |
| | GTLVTVSS (SEQ ID NO: 141) |
| | HCDR1*: FTFSSFGMS (SEQ ID NO: 128) |
| | HCDR1: SFGMS (SEQ ID NO: 129) |
| | HCDR2: SISGSGSDTLYADSVRG (SEQ ID NO: 124) |
| | HCDR3*: SIGGSLIR (SEQ ID NO: 142) |
| | HCDR3: GGSLIR (SEQ ID NO: 143) |
| CNG-HSA- | KVQLVESGGGLVQPGGSLRLSCAASGFTFGSFGMSWVRQAPGKGLEWVSSISG |
| 108 | SGADTLYADSVKGRFTISRDNSKNTLYLQMNSLRAEDTAVYYCTIGGSLRASS |
| | QGTLVTVSS (SEQ ID NO: 144) |
| | HCDR1*: FTFGSFGMS (SEQ ID NO: 145) |
| | HCDR1: SFGMS (SEQ ID NO: 129) |
| | HCDR2: SISGSGADTLYADSVKG (SEQ ID NO: 146) |
| | HCDR3*: TIGGSLRA (SEQ ID NO: 147) |
| | HCDR3: GGSLRA (SEQ ID NO: 148) |
| CNG-HSA- | KVQLVESGGGLVQPGNSLRLSCAASGFTFGSFGMSWVRQAPGKGPEWVSSISG |
| 109 | SGSDTLYADSVKGRFTISRDNSKNTLYLQMNSLRPEDTAVYYCTIGGSLSRSSQ |
| | GTLVTVSS (SEQ ID NO: 149) |
| | HCDR1*: FTFGSFGMS (SEQ ID NO: 145) |
| | HCDR1: SFGMS (SEQ ID NO: 129) |
| | HCDR2: SISGSGSDTLYADSVKG (SEQ ID NO: 133) |
| | HCDR3*: TIGGSLSR (SEQ ID NO: 134) |
| | HCDR3: GGSLSR (SEQ ID NO: 135) |
| CNG-HSA- | KVQLVESGGGLVQPGNSLRLSCAASGFTFSSFGMSWVRQAPGKGLEWVSSISG |
| 110 | SGGDTLYADSAKGRFTISRDNSKNTLYLQMNSLRPEDTAVYYCTIGGSLSRSSQ |
| | GTLVTVSS (SEQ ID NO: 150) |
| | HCDR1*: FTFSSFGMS (SEQ ID NO: 128) |
| | HCDR1: SFGMS (SEQ ID NO: 129) |
| | HCDR2: SISGSGGDTLYADSAKG (SEQ ID NO: 151) |
| | HCDR3*: TIGGSLSR (SEQ ID NO: 134) |
| | HCDR3: GGSLSR (SEQ ID NO: 135) |
| CNG-HSA- | KVQLVESGGGLVQPGGSLRLSCAASGFTFSSFGMSWVRQAPGKGLEWVSSISG |
| 111 | SGSDTLYADSVEGRFTISRDNSKNTLYLQMNSLRAEDTAVYYCTIGGSLKQSSQ |
| | GTLVTVSS (SEQ ID NO: 152) |
| | HCDR1*: FTFSSFGMS (SEQ ID NO: 128) |
| | HCDR1: SFGMS (SEQ ID NO: 129) |
| | HCDR2: SISGSGSDTLYADSVEG (SEQ ID NO: 153) |
| | HCDR3*: TIGGSLKQ (SEQ ID NO: 154) |
| | HCDR3: GGSLKQ (SEQ ID NO: 155) |
| CNG-HSA- | KVQLLESGGGLVQPGGSLRLSCAASGFTFPSFGMSWVRQAPGKGLEWVSSISG |
| 112 | SGSDTLYADSVKGRFTISRDNAKTTLYLQMNSLRPEDTAVYYCTIGGSVSKSSR |
| | GTLVTVSS (SEQ ID NO: 156) |
| | HCDR1*: FTFPSFGMS (SEQ ID NO: 157) |
| | HCDR1: SFGMS (SEQ ID NO: 129) |
| | HCDR2: SISGSGSDTLYADSVKG (SEQ ID NO: 133) |
| | HCDR3*: TIGGSVSK (SEQ ID NO: 158) |
| | HCDR3: GGSVSK (SEQ ID NO: 159) |
| CNG-HSA- | GVQLLESGGGLVQPGGSLRLSCAASGFTFSSFGMSWVRQAPGKGLEWVSSISG |
| 113 | TGSDTLYADSVKGRFTISRDNSKNTLYLQMNSLRPEDTAVYYCTIGGSLRYSSQ |
| | GTLVTVSS (SEQ ID NO: 160) |
| | HCDR1*: FTFSSFGMS (SEQ ID NO: 128) |
| | HCDR1: SFGMS (SEQ ID NO: 129) |
| | HCDR2: SISGTGSDTLYADSVKG (SEQ ID NO: 161) |
| | HCDR3*: TIGGSLRY (SEQ ID NO: 162) |
| | HCDR3: GGSLRY (SEQ ID NO: 163) |
| CNG-HSA- | KVQLVESGGGLVQPGNSLRLSCAASGFTFSSFGMSWVRQAPGKGLEWVSSISG |
| 114 | SGSDTLTADSVKGRFTISRDNSKNTLYLQMNSLRAEDTAVYYCTIGGSLVRSSQ |
| | GTLVTVSS (SEQ ID NO: 164) |
| | HCDR1*: FTFSSFGMS (SEQ ID NO: 128) |
| | HCDR1: SFGMS (SEQ ID NO: 129) |
| | HCDR2: SISGSGSDTLTADSVKG (SEQ ID NO: 165) |
| | HCDR3*: TIGGSLVR (SEQ ID NO: 166) |
| | HCDR3: GGSLVR (SEQ ID NO: 167) |
| CNG-HSA- | KVQLVESGGGLVQPGNSLRLSCAASGFTFSSFGMGWVRQAPGKGLEWVSSISG |
| 115 | SGSDTLYAPSVKGRFTISRDNSKNTLYLQMNSLRPEDTAVYYCTFAGSLSRSSQ |
| | GTLVTVSS (SEQ ID NO: 168) |
| | HCDR1*: FTFSSFGMG (SEQ ID NO: 169) |
| | HCDR1: SFGMG (SEQ ID NO: 170) |
| | HCDR2: SISGSGSDTLYAPSVKG (SEQ ID NO: 171) |
| | HCDR3*: TFAGSLSR (SEQ ID NO: 172) |
| | HCDR3: AGSLSR (SEQ ID NO: 173) |
| CNG-HSA- | KVQLVESGGGLVQPGGSLRLSCAASGFTFSSFGMSWVRQAPGKGPEWVSSISG |
| 116 | SGSDTLYADSVKGRFTISRDNSKNTLYLQMNSLRAEDTAVYYCTIGGSLRASSQ |
| | GTLVTVSS (SEQ ID NO: 174) |
| | HCDR1*: FTFSSFGMS (SEQ ID NO: 128) |
| | HCDR1: SFGMS (SEQ ID NO: 129) |
| | HCDR2: SISGSGSDTLYADSVKG (SEQ ID NO: 133) |
| | HCDR3*: TIGGSLRA (SEQ ID NO: 147) |
| | HCDR3: GGSLRA (SEQ ID NO: 148) |
| CNG-HSA- | KVQLVESGGGLVQPGNSLRLSCAASGFTFSSFGMSWVRQAPGKGLEWVSSISG |
| 117 | SGSDTLYADSVKGRFTISRDNSKNTLYLQMNSLRAEDTAVYYCTIGGSLTRSSQ |
| | GTLVTVSS (SEQ ID NO: 175) |
| | HCDR1*: FTFSSFGMS (SEQ ID NO: 128) |
| | HCDR1: SFGMS (SEQ ID NO: 129) |
| | HCDR2: SISGSGSDTLYADSVKG (SEQ ID NO: 133) |
| | HCDR3*: TIGGSLTR (SEQ ID NO: 176) |
| | HCDR3: GGSLTR (SEQ ID NO: 177) |
| CNG-HSA- | KVQLVESGGGLVQPGNSLRLSCAASGFTFSSFGMSWVRQAPGKGLEWVSSISG |
| 118 | GGSDTLYADSVRGRFTISRDNSKNTLYLQMNSLRAEDTAVYYCTIGGSLRASS |
| | QGTLVTVSS (SEQ ID NO: 178) |
| | HCDR1*: FTFSSFGMS (SEQ ID NO: 128) |
| | HCDR1: SFGMS (SEQ ID NO: 129) |
| | HCDR2: SISGGGSDTLYADSVRG (SEQ ID NO: 179) |
| | HCDR3*: TIGGSLRA (SEQ ID NO: 147) |
| | HCDR3: GGSLRA (SEQ ID NO: 148) |
| CNG-HSA- | KVQLVESGGGLVQPGNSLRLSCAASGFTFSSFGMSWVRQAPGKGLEWVSSISG |
| 119 | SGSDALYADSAKGRFTISRDNAKTTLYLQMNSLRAEDTAVYYCTIGGSLSPSSQ |
| | GTLVTVSS (SEQ ID NO: 180) |
| | HCDR1*: FTFSSFGMS (SEQ ID NO: 128) |
| | HCDR1: SFGMS (SEQ ID NO: 129) |
| | HCDR2: SISGSGSDALYADSAKG (SEQ ID NO: 181) |
| | HCDR3*: TIGGSLSP (SEQ ID NO: 125) |
| | HCDR3: GGSLSP (SEQ ID NO: 126) |
| CNG-HSA- | KVQLVESGGGLVQPGGSLRLSCAASGFTFSSFGMSWVRQAPGKGPEWVSSISG |
| 120 | SGSDTLYADSVKGRFTISRDNSKNTLYLQMNSLRPEDTAVYYCTIGGSLKQSSQ |
| | GTLVTVSS (SEQ ID NO: 182) |
| | HCDR1*: FTFSSFGMS (SEQ ID NO: 128) |
| | HCDR1: SFGMS (SEQ ID NO: 129) |
| | HCDR2: SISGSGSDTLYADSVKG (SEQ ID NO: 133) |
| | HCDR3*: TIGGSLKQ (SEQ ID NO: 154) |
| | HCDR3: GGSLKQ (SEQ ID NO: 155) |
| Consensus-1 | HCDR1*: FX₁FX₂SFGMX₃, wherein X₁ is T or V; X₂ is S, H, G, or P; and X₃ is S, T, |
| (CNG-HSA- | or G (SEQ ID NO: 408) |
| 101 to -120) | HCDR1: SFGMX, whereinX is S, T, or G (SEQ ID NO: 184) |
| | HCDR2: SISGX₁GX₂DX₃LX₄AX₅SX₆X₇G, wherein X₁ is S, T, or G; X₂ is S, A, or G; |
| | X₃ is T or A; X₄ is Y or T; X₅ is D or P; X₆ is V or A; and X₇ is K, R, or E |
| | (SEQ ID NO: 409) |
| | HCDR3*: X₁X₂X₃GSX₄X₅X₆, wherein X₁ is T or S; X₂ is I or F; X₃ is G or A; X₄ is L, |
| | R, or V; X₅ is S, I, R, K, V, or T; and X₆ is R, P, I, A, Q, K, or Y |
| | (SEQ ID NO: 410) |
| | HCDR3: X₁GSX₂X₃X₄, wherein X₁ is G or A; X₂ is L, R, or V; X₃ is S, I, R, K, V, |
| | or T; and X₄ is R, P, I, A, Q, K, or Y (SEQ ID NO: 411) |
| Consensus-2 | HCDR1*: FTFX₁SFGMX₂, wherein X₁ is S, H, G, or P; and X₂ is S, T, or G (SEQ ID |
| (CNG-HSA- | NO: 183) |
| 101 to -104, | HCDR1: SFGMX, wherein X is S, T, or G (SEQ ID NO: 184) |
| -108 to-112, | HCDR2: SISGX₁GX₂DX₃LYAX₄SX₅X₆G, wherein X₁ is S or G; X₂ is S, A, or G; X₃ is |
| and -115 to | T or A; X₄ is D or P; X₅ is V or A; and X₆ is K, R, or E (SEQ ID NO: 185) |
| -120) | HCDR3*: TX₁X₂GSX₃X₄X₅, wherein X₁ is I or F; X₂ is G or A; X₃ is L or V; X₄ is S, |
| | R, K, or T; and X₅ is R, P, A, Q, or K (SEQ ID NO: 186) |
| | HCDR3: X₁GSX₂X₃X₄, wherein X₁ is G or A; X₂ is L or V; X₃ is S, R, K, or T; and |
| | X₄ is R, P, A, Q, or K (SEQ ID NO: 187) |
| Consensus-3 | HCDR1*: FTFX₁SFGMX₂, wherein X₁ is S, H, or G; and X₂ is S or T (SEQ ID NO: |
| (CNG-HSA- | 188) |
| 101, -102, | HCDR1: SFGMX, wherein X is S or T (SEQ ID NO: 189) |
| -104, -109, | HCDR2: SISGX₁GSDTLYADSVX₂G, wherein X₁ is S or T; and X₂ is K or R (SEQ ID |
| -113, | NO: 190) |
| -116, and-117) | HCDR3*: TIGGSLX₁X₂, wherein X₁ is S, R, or T; and X₂ is R, P, Y, or A (SEQ ID |
| | NO: 191) |
| | HCDR3: GGSLX₁X₂, wherein X₁ is S, R, or T; and X₂ is R, P, Y, or A (SEQ ID NO: |
| | 192) |
| Consensus-4 | HCDR1*: FTFX₁SFGMX₂, wherein X₁ is S, H, or G; and X₂ is S or T (SEQ ID NO: |
| (CNG-HSA- | 188) |
| 101, -102, | HCDR1: SFGMX, wherein X is S or T (SEQ ID NO: 189) |
| -104, -109, | HCDR2: SISGSGSDTLYADSVXG, wherein X is K or R (SEQ ID NO: 193) |
| -116, and-117) | HCDR3*: TIGGSLXX2, wherein X₁ is S, R, or T; and X₂ is R, P, or A (SEQ ID NO: |
| | 194) |
| | HCDR3: GGSLX₁X₂, wherein X₁ is S, R, or T; and X₂ is R, P, or A (SEQ ID NO: 195) |

In certain embodiments, the antigen-binding site that binds serum albumin of the present invention comprises a VH that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VH of an antibody disclosed in Table 1. In certain embodiments, the antigen-binding site comprises the HCDR1, HCDR2, and HCDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), IMGT (see Lefranc, (1999) The Immunologist, 7, 132-136), or any other CDR determination method known in the art, of the VH sequence of an antibody disclosed in Table 1. In certain embodiments, the antigen-binding site comprises the HCDR1, HCDR2, and HCDR3 sequences of an antibody disclosed in Table 1. In certain embodiments, the antigen-binding site comprises the VH sequence of an antibody disclosed in Table 1.

### Series 1 Constructs

In certain embodiments, the antigen-binding site that binds HSA comprises a VH comprising HCDR1, HCDR2, and HCDR3 sequences set forth in SEQ ID NOs: 408, 409, and 410, respectively, wherein the antigen-binding site does not comprise HCDR1, HCDR2, and HCDR3, sequences set forth in SEQ ID NOs: 128, 133, and 134, respectively. In certain embodiments, the HCDR1 sequence is selected from the group consisting of SEQ ID NOs: 122, 128, 132, 137, 145, 157, and 169; the HCDR2 sequence is selected from the group consisting of SEQ ID NOs: 124, 133, 146, 151, 153, 161, 165, 171, 179, and 181; and/or the HCDR3 sequence is selected from the group consisting of SEQ ID NOs: 125, 134, 142, 147, 154, 158, 162, 166, 172, and 176.

In certain embodiments, the antigen-binding site comprises a VH comprising HCDR1, HCDR2, and HCDR3 sequences set forth in SEQ ID NOs: 184, 409, and 411, respectively, wherein the antigen-binding site does not comprise HCDR1, HCDR2, and HCDR3 sequences set forth in SEQ ID NOs: 129, 133, and 135, respectively. In certain embodiments, the HCDR1 sequence is selected from the group consisting of SEQ ID NOs: 123, 129, and 170; the HCDR2 sequence is selected from the group consisting of SEQ ID NOs: 124, 133, 146, 151, 153, 161, 165, 171, 179, and 181; and/or the HCDR3 sequence is selected from the group consisting of SEQ ID NOs: 126, 135, 143, 148, 155, 159, 163, 167, 173, and 177.

In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121.

In certain embodiments, the antigen-binding site has a higher binding affinity to human serum albumin, cynomolgus serum albumin, mouse serum albumin, and/or protein A relative to an antigen-binding site having VH sequence set forth in SEQ ID NO: 196.

### Series 2 Constructs

In certain embodiments, the antigen-binding site that binds HSA comprises a VH comprising HCDR1, HCDR2, and HCDR3 sequences set forth in SEQ ID NOs: 183, 185, and 186, respectively, wherein the antigen-binding site does not comprise HCDR1, HCDR2, and HCDR3, sequences set forth in SEQ ID NOs: 128, 133, and 134, respectively. In certain embodiments, the HCDR1 sequence is selected from the group consisting of SEQ ID NOs: 122, 128, 132, 145, 157, and 169; the HCDR2 sequence is selected from the group consisting of SEQ ID NOs: 124, 133, 146, 151, 153, 171, 179, and 181; and/or the HCDR3 sequence is selected from the group consisting of SEQ ID NOs: 125, 134, 147, 154, 158, 172, and 176.

In certain embodiments, the antigen-binding site comprises a VH comprising HCDR1, HCDR2, and HCDR3 sequences set forth in SEQ ID NOs: 184, 185, and 187, respectively, wherein the antigen-binding site does not comprise HCDR1, HCDR2, and HCDR3 sequences set forth in SEQ ID NOs: 129, 133, and 135, respectively. In certain embodiments, the HCDR1 sequence is selected from the group consisting of SEQ ID NOs: 123, 129, and 170; the HCDR2 sequence is selected from the group consisting of SEQ ID NOs: 124, 133, 146, 151, 153, 171, 179, and 181; and/or the HCDR3 sequence is selected from the group consisting of SEQ ID NOs: 126, 135, 148, 155, 159, 173, and 177.

In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121.

In certain embodiments, the antigen-binding site has a higher binding affinity to human serum albumin relative to an antigen-binding site having VH sequence set forth in SEQ ID NO: 196. In certain embodiments, the antigen-binding site binds human serum albumin with a K_{D} lower than or equal to 10 nM, 9 nM, 8 nM, 7 nM, 6 nM, 5 nM, 4 nM, or 3 nM, as measured by SPR when the antigen-binding site is present as a monomer. In certain embodiments, the antigen-binding site binds human serum albumin with a K_{D} in the range of 1-10 nM, 1-9 nM, 1-8 nM, 1-7 nM, 1-6 nM, 1-5 nM, 1-4 nM, or 1-3 nM, as measured by SPR when the antigen-binding site is present as a monomer.

### Series 3 Constructs

In certain embodiments, the antigen-binding site that binds HSA comprises a VH comprising HCDR1, HCDR2, and HCDR3 sequences set forth in SEQ ID NOs: 188, 190, and 191, respectively, wherein the antigen-binding site does not comprise HCDR1, HCDR2, and HCDR3, sequences set forth in SEQ ID NOs: 128, 133, and 134, respectively. In certain embodiments, the HCDR1 sequence is selected from the group consisting of SEQ ID NOs: 122, 128, 132, and 145; the HCDR2 sequence is selected from the group consisting of SEQ ID NOs: 124, 133, and 161; and/or the HCDR3 sequence is selected from the group consisting of SEQ ID NOs: 125, 134, 162, 147, and 176.

In certain embodiments, the antigen-binding site comprises a VH comprising HCDR1, HCDR2, and HCDR3 sequences set forth in SEQ ID NOs: 189, 190, and 192, respectively, wherein the antigen-binding site does not comprise HCDR1, HCDR2, and HCDR3 sequences set forth in SEQ ID NOs: 129, 133, and 135, respectively. In certain embodiments, the HCDR1 sequence is selected from the group consisting of SEQ ID NOs: 123 and 129; the HCDR2 sequence is selected from the group consisting of SEQ ID NOs: 124, 133, and 161; and/or the HCDR3 sequence is selected from the group consisting of SEQ ID NOs: 126, 135, 163, 148, and 177.

In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121.

In certain embodiments, the antigen-binding site has a higher binding affinity to protein A relative to an antigen-binding site having VH sequence set forth in SEQ ID NO: 196. In certain embodiments, the antigen-binding site binds protein A with a K_{D} lower than or equal to 2.5 nM or 2 nM, as measured by SPR when the antigen-binding site is present as a monomer. In certain embodiments, the antigen-binding site binds protein A with a K_{D} in the range of 1-2.5 nM or 1-2 nM, as measured by SPR when the antigen-binding site is present as a monomer.

### Series 4 Constructs

In certain embodiments, the antigen-binding site that binds HSA comprises a VH comprising HCDR1, HCDR2, and HCDR3 sequences set forth in SEQ ID NOs: 188, 193, and 194, respectively, wherein the antigen-binding site does not comprise HCDR1, HCDR2, and HCDR3, sequences set forth in SEQ ID NOs: 128, 133, and 134, respectively. In certain embodiments, the HCDR1 sequence is selected from the group consisting of SEQ ID NOs: 122, 128, 132, and 145; the HCDR2 sequence is selected from the group consisting of SEQ ID NOs: 124 and 133; and/or the HCDR3 sequence is selected from the group consisting of SEQ ID NOs: 125, 134, 147, and 176.

In certain embodiments, the antigen-binding site comprises a VH comprising HCDR1, HCDR2, and HCDR3 sequences set forth in SEQ ID NOs: 189, 193, and 195, respectively, wherein the antigen-binding site does not comprise HCDR1, HCDR2, and HCDR3 sequences set forth in SEQ ID NOs: 129, 133, and 135, respectively. In certain embodiments, the HCDR1 sequence is selected from the group consisting of SEQ ID NOs: 123 and 129; the HCDR2 sequence is selected from the group consisting of SEQ ID NOs: 124 and 133; and/or the HCDR3 sequence is selected from the group consisting of SEQ ID NOs: 126, 135, 148, and 177.

In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121.

In certain embodiments, the antigen-binding site has a higher binding affinity to human serum albumin and a higher affinity to protein A relative to an antigen-binding site having VH sequence set forth in SEQ ID NO: 196. In certain embodiments, the antigen-binding site binds human serum albumin with a K_{D} lower than or equal to 10 nM, 9 nM, 8 nM, 7 nM, 6 nM, 5 nM, 4 nM, or 3 nM and binds protein A with a K_{D} lower than or equal to 2.5 nM or 2 nM, as measured by SPR when the antigen-binding site is present as a monomer. In certain embodiments, the antigen-binding site binds human serum albumin with a K_{D} in the range of 1-10 nM, 1-9 nM, 1-8 nM, 1-7 nM, 1-6 nM, 1-5 nM, 1-4 nM, or 1-3 nM and binds protein A with a K_{D} in the range of 1-2.5 nM or 1-2 nM, as measured by SPR when the antigen-binding site is present as a monomer.

### Individual Constructs

In certain embodiments, the antigen-binding site that binds serum albumin is derived from CNG-HSA-101. In certain embodiments, the antigen-binding site comprises a VH comprising HCDR1, HCDR2, and HCDR3 sequences set forth in SEQ ID NOs: 122, 124, and 125, respectively. In certain embodiments, the antigen-binding site comprises a VH comprising HCDR1, HCDR2, and HCDR3 sequences set forth in SEQ ID NOs: 123, 124, and 126, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 121. In certain embodiments, the VH comprises the amino acid sequence of SEQ ID NO: 121.

In certain embodiments, the antigen-binding site that binds serum albumin is derived from CNG-HSA-102. In certain embodiments, the antigen-binding site comprises a VH comprising HCDR1, HCDR2, and HCDR3 sequences set forth in SEQ ID NOs: 128, 124, and 125, respectively. In certain embodiments, the antigen-binding site comprises a VH comprising HCDR1, HCDR2, and HCDR3 sequences set forth in SEQ ID NOs: 129, 124, and 126, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 127. In certain embodiments, the VH comprises the amino acid sequence of SEQ ID NO: 127.

In certain embodiments, the antigen-binding site that binds serum albumin is derived from CNG-HSA-103. In certain embodiments, the antigen-binding site comprises a VH comprising HCDR1, HCDR2, and HCDR3 sequences set forth in SEQ ID NOs: 122, 124, and 125, respectively. In certain embodiments, the antigen-binding site comprises a VH comprising HCDR1, HCDR2, and HCDR3 sequences set forth in SEQ ID NOs: 123, 124, and 126, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 130. In certain embodiments, the VH comprises the amino acid sequence of SEQ ID NO: 130.

In certain embodiments, the antigen-binding site that binds serum albumin is derived from CNG-HSA-104. In certain embodiments, the antigen-binding site comprises a VH comprising HCDR1, HCDR2, and HCDR3 sequences set forth in SEQ ID NOs: 132, 133, and 134, respectively. In certain embodiments, the antigen-binding site comprises a VH comprising HCDR1, HCDR2, and HCDR3 sequences set forth in SEQ ID NOs: 129, 133, and 135, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 131. In certain embodiments, the VH comprises the amino acid sequence of SEQ ID NO: 131.

In certain embodiments, the antigen-binding site that binds serum albumin is derived from CNG-HSA-105. In certain embodiments, the antigen-binding site comprises a VH comprising HCDR1, HCDR2, and HCDR3 sequences set forth in SEQ ID NOs: 137, 133, and 134, respectively. In certain embodiments, the antigen-binding site comprises a VH comprising HCDR1, HCDR2, and HCDR3 sequences set forth in SEQ ID NOs: 129, 133, and 135, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 136. In certain embodiments, the VH comprises the amino acid sequence of SEQ ID NO: 136.

In certain embodiments, the antigen-binding site that binds serum albumin is derived from CNG-HSA-106. In certain embodiments, the antigen-binding site comprises a VH comprising HCDR1, HCDR2, and HCDR3 sequences set forth in SEQ ID NOs: 128, 124, and 139, respectively. In certain embodiments, the antigen-binding site comprises a VH comprising HCDR1, HCDR2, and HCDR3 sequences set forth in SEQ ID NOs: 129, 124, and 140, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 138. In certain embodiments, the VH comprises the amino acid sequence of SEQ ID NO: 138.

In certain embodiments, the antigen-binding site that binds serum albumin is derived from CNG-HSA-107. In certain embodiments, the antigen-binding site comprises a VH comprising HCDR1, HCDR2, and HCDR3 sequences set forth in SEQ ID NOs: 128, 124, and 142, respectively. In certain embodiments, the antigen-binding site comprises a VH comprising HCDR1, HCDR2, and HCDR3 sequences set forth in SEQ ID NOs: 129, 124, and 143, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 141. In certain embodiments, the VH comprises the amino acid sequence of SEQ ID NO: 141.

In certain embodiments, the antigen-binding site that binds serum albumin is derived from CNG-HSA-108. In certain embodiments, the antigen-binding site comprises a VH comprising HCDR1, HCDR2, and HCDR3 sequences set forth in SEQ ID NOs: 145, 146, and 147, respectively. In certain embodiments, the antigen-binding site comprises a VH comprising HCDR1, HCDR2, and HCDR3 sequences set forth in SEQ ID NOs: 129, 146, and 148, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 144. In certain embodiments, the VH comprises the amino acid sequence of SEQ ID NO: 144.

In certain embodiments, the antigen-binding site that binds serum albumin is derived from CNG-HSA-109. In certain embodiments, the antigen-binding site comprises a VH comprising HCDR1, HCDR2, and HCDR3 sequences set forth in SEQ ID NOs: 145, 133, and 134, respectively. In certain embodiments, the antigen-binding site comprises a VH comprising HCDR1, HCDR2, and HCDR3 sequences set forth in SEQ ID NOs: 129, 133, and 135, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 149. In certain embodiments, the VH comprises the amino acid sequence of SEQ ID NO: 149.

In certain embodiments, the antigen-binding site that binds serum albumin is derived from CNG-HSA-110. In certain embodiments, the antigen-binding site comprises a VH comprising HCDR1, HCDR2, and HCDR3 sequences set forth in SEQ ID NOs: 128, 151, and 134, respectively. In certain embodiments, the antigen-binding site comprises a VH comprising HCDR1, HCDR2, and HCDR3 sequences set forth in SEQ ID NOs: 129, 151, and 135, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 150. In certain embodiments, the VH comprises the amino acid sequence of SEQ ID NO: 150.

In certain embodiments, the antigen-binding site that binds serum albumin is derived from CNG-HSA-111. In certain embodiments, the antigen-binding site comprises a VH comprising HCDR1, HCDR2, and HCDR3 sequences set forth in SEQ ID NOs: 128, 153, and 154, respectively. In certain embodiments, the antigen-binding site comprises a VH comprising HCDR1, HCDR2, and HCDR3 sequences set forth in SEQ ID NOs: 129, 153, and 155, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 152. In certain embodiments, the VH comprises the amino acid sequence of SEQ ID NO: 152.

In certain embodiments, the antigen-binding site that binds serum albumin is derived from CNG-HSA-112. In certain embodiments, the antigen-binding site comprises a VH comprising HCDR1, HCDR2, and HCDR3 sequences set forth in SEQ ID NOs: 157, 133, and 158, respectively. In certain embodiments, the antigen-binding site comprises a VH comprising HCDR1, HCDR2, and HCDR3 sequences set forth in SEQ ID NOs: 129, 133, and 159, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 156. In certain embodiments, the VH comprises the amino acid sequence of SEQ ID NO: 156.

In certain embodiments, the antigen-binding site that binds serum albumin is derived from CNG-HSA-113. In certain embodiments, the antigen-binding site comprises a VH comprising HCDR1, HCDR2, and HCDR3 sequences set forth in SEQ ID NOs: 128, 161, and 162, respectively. In certain embodiments, the antigen-binding site comprises a VH comprising HCDR1, HCDR2, and HCDR3 sequences set forth in SEQ ID NOs: 129, 161, and 163, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 160. In certain embodiments, the VH comprises the amino acid sequence of SEQ ID NO: 160.

In certain embodiments, the antigen-binding site that binds serum albumin is derived from CNG-HSA-114. In certain embodiments, the antigen-binding site comprises a VH comprising HCDR1, HCDR2, and HCDR3 sequences set forth in SEQ ID NOs: 128, 165, and 166, respectively. In certain embodiments, the antigen-binding site comprises a VH comprising HCDR1, HCDR2, and HCDR3 sequences set forth in SEQ ID NOs: 129, 165, and 167, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 164. In certain embodiments, the VH comprises the amino acid sequence of SEQ ID NO: 164.

In certain embodiments, the antigen-binding site that binds serum albumin is derived from CNG-HSA-115. In certain embodiments, the antigen-binding site comprises a VH comprising HCDR1, HCDR2, and HCDR3 sequences set forth in SEQ ID NOs: 169, 171, and 172, respectively. In certain embodiments, the antigen-binding site comprises a VH comprising HCDR1, HCDR2, and HCDR3 sequences set forth in SEQ ID NOs: 170, 171, and 173, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 168. In certain embodiments, the VH comprises the amino acid sequence of SEQ ID NO: 168.

In certain embodiments, the antigen-binding site that binds serum albumin is derived from CNG-HSA-116. In certain embodiments, the antigen-binding site comprises a VH comprising HCDR1, HCDR2, and HCDR3 sequences set forth in SEQ ID NOs: 128, 133, and 147, respectively. In certain embodiments, the antigen-binding site comprises a VH comprising HCDR1, HCDR2, and HCDR3 sequences set forth in SEQ ID NOs: 129, 133, and 148, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 174. In certain embodiments, the VH comprises the amino acid sequence of SEQ ID NO: 174.

In certain embodiments, the antigen-binding site that binds serum albumin is derived from CNG-HSA-117. In certain embodiments, the antigen-binding site comprises a VH comprising HCDR1, HCDR2, and HCDR3 sequences set forth in SEQ ID NOs: 128, 133, and 176, respectively. In certain embodiments, the antigen-binding site comprises a VH comprising HCDR1, HCDR2, and HCDR3 sequences set forth in SEQ ID NOs: 129, 133, and 177, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 175. In certain embodiments, the VH comprises the amino acid sequence of SEQ ID NO: 175.

In certain embodiments, the antigen-binding site that binds serum albumin is derived from CNG-HSA-118. In certain embodiments, the antigen-binding site comprises a VH comprising HCDR1, HCDR2, and HCDR3 sequences set forth in SEQ ID NOs: 128, 179, and 147, respectively. In certain embodiments, the antigen-binding site comprises a VH comprising HCDR1, HCDR2, and HCDR3 sequences set forth in SEQ ID NOs: 129, 179, and 148, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 178. In certain embodiments, the VH comprises the amino acid sequence of SEQ ID NO: 178.

In certain embodiments, the antigen-binding site that binds serum albumin is derived from CNG-HSA-119. In certain embodiments, the antigen-binding site comprises a VH comprising HCDR1, HCDR2, and HCDR3 sequences set forth in SEQ ID NOs: 128, 181, and 125, respectively. In certain embodiments, the antigen-binding site comprises a VH comprising HCDR1, HCDR2, and HCDR3 sequences set forth in SEQ ID NOs: 129, 181, and 126, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 180. In certain embodiments, the VH comprises the amino acid sequence of SEQ ID NO: 180.

In certain embodiments, the antigen-binding site that binds serum albumin is derived from CNG-HSA-120. In certain embodiments, the antigen-binding site comprises a VH comprising HCDR1, HCDR2, and HCDR3 sequences set forth in SEQ ID NOs: 128, 133, and 154, respectively. In certain embodiments, the antigen-binding site comprises a VH comprising HCDR1, HCDR2, and HCDR3 sequences set forth in SEQ ID NOs: 129, 133, and 155, respectively. In certain embodiments, the antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 180. In certain embodiments, the VH comprises the amino acid sequence of SEQ ID NO: 180.

In certain embodiments, the antigen-binding site derived from CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, or CNG-HSA-120 has a higher binding affinity to human, cynomolgus, and/or mouse serum albumin relative to an antigen-binding site having VH sequence set forth in SEQ ID NO: 196.

In certain embodiments, the antigen-binding site derived from CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, or CNG-HSA-120 binds human serum albumin with a K_{D} lower than or equal to 10 nM, 9 nM, 8 nM, 7 nM, 6 nM, 5 nM, 4 nM, or 3 nM, as measured by SPR when the antigen-binding site is present as a monomer. In certain embodiments, the antigen-binding site derived from CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, or CNG-HSA-120 binds human serum albumin with a K_{D} in the range of 1-10 nM, 1-9 nM, 1-8 nM, 1-7 nM, 1-6 nM, 1-5 nM, 1-4 nM, or 1-3 nM, as measured by SPR when the antigen-binding site is present as a monomer.

In certain embodiments, the antigen-binding site derived from CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, or CNG-HSA-120 binds cynomolgus serum albumin with a K_{D} lower than or equal to 9 nM, 8 nM, 7 nM, 6 nM, 5 nM, 4 nM, or 3 nM, as measured by SPR when the antigen-binding site is present as a monomer. In certain embodiments, the antigen-binding site derived from CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, or CNG-HSA-120 binds cynomolgus serum albumin with a K_{D} in the range of 1-9 nM, 1-8 nM, 1-7 nM, 1-6 nM, 1-5 nM, 1-4 nM, or 1-3 nM, as measured by SPR when the antigen-binding site is present as a monomer.

In certain embodiments, the antigen-binding site derived from CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, or CNG-HSA-120 binds mouse serum albumin with a K_{D} lower than or equal to 100 nM, 90 nM, 80 nM, 70 nM, 60 nM, 50 nM, 40 nM, 30 nM, 20 nM, or 10 nM, as measured by SPR when the antigen-binding site is present as a monomer. In certain embodiments, the antigen-binding site derived from CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, or CNG-HSA-120 binds mouse serum albumin with a K_{D} in the range of 1-100 nM, 1-90 nM, 1-80 nM, 1-70 nM, 1-60 nM, 1-50 nM, 1-40 nM, 1-30 nM, 1-20 nM, or 1-10 nM, as measured by SPR when the antigen-binding site is present as a monomer.

In certain embodiments, the antigen-binding site derived from CNG-HSA-101, CNG-HSA-103, CNG-HSA-106, CNG-HSA-107, CNG-HSA-108, CNG-HSA-109, CNG-HSA-111, CNG-HSA-113, CNG-HSA-114, CNG-HSA-115, CNG-HSA-116, CNG-HSA-118, or CNG-HSA-120 binds human serum albumin with a first K_{D} and binds mouse serum albumin with a second K_{D}, wherein the ratio of the second K_{D} to the first K_{D} is in the range of 0.5-10, 0.5-9, 0.5-8, 0.5-7, 0.5-6, 0.5-5, 0.5-4, 0.5-3, 0.5-2, 0.9-10, 0.9-9, 0.9-8, 0.9-7, 0.9-6, 0.9-5, 0.9-4, 0.9-3, 0.9-2, 1-10, 1-9, 1-8, 1-7, 1-6, 1-5, 1-4, 1-3, or 1-2. It is understood that an antigen-binding site having a ratio closer to 1 has more similar affinity to mouse serum albumin relative to affinity to human serum albumin, which allows assessment of the pharmacokinetics of the antigen-binding site or a protein comprising the same using a mouse model at higher accuracy.

In certain embodiments, the antigen-binding site derived from CNG-HSA-101, CNG-HSA-102, CNG-HSA-104, CNG-HSA-109, CNG-HSA-113, CNG-HSA-116, or CNG-HSA-117 has a higher binding affinity to protein A relative to an antigen-binding site having VH sequence set forth in SEQ ID NO: 196. It is understood that increased affinity to protein A allows purification of the antigen-binding site, or a protein that comprises the antigen-binding site but not an antibody Fc region, by protein A chromatography. In certain embodiments, the antigen-binding site derived from CNG-HSA-101, CNG-HSA-102, CNG-HSA-104, CNG-HSA-109, CNG-HSA-113, CNG-HSA-116, or CNG-HSA-117 binds human serum albumin with a K_{D} lower than or equal to 10 nM, 9 nM, 8 nM, 7 nM, 6 nM, 5 nM, 4 nM, or 3 nM and binds protein A with a K_{D} lower than or equal to 2.5 nM or 2 nM, as measured by SPR when the antigen-binding site is present as a monomer. In certain embodiments, the antigen-binding site derived from CNG-HSA-101, CNG-HSA-102, CNG-HSA-104, CNG-HSA-109, CNG-HSA-113, CNG-HSA-116, or CNG-HSA-117 binds human serum albumin with a K_{D} in the range of 1-10 nM, 1-9 nM, 1-8 nM, 1-7 nM, 1-6 nM, 1-5 nM, 1-4 nM, or 1-3 nM and binds protein A with a K_{D} in the range of 1-2.5 nM or 1-2 nM, as measured by SPR when the antigen-binding site is present as a monomer.

In certain embodiments, the antigen-binding site derived from CNG-HSA-101, CNG-HSA-102, CNG-HSA-104, CNG-HSA-109, CNG-HSA-116, or CNG-HSA-117 has a higher binding affinity to human serum albumin and a higher affinity to protein A relative to an antigen-binding site having VH sequence set forth in SEQ ID NO: 196. In certain embodiments, the antigen-binding site derived from CNG-HSA-101, CNG-HSA-102, CNG-HSA-104, CNG-HSA-109, CNG-HSA-116, or CNG-HSA-117 binds protein A with a K_{D} lower than or equal to 2.5 nM or 2 nM, as measured by SPR when the antigen-binding site is present as a monomer. In certain embodiments, the antigen-binding site derived from CNG-HSA-101, CNG-HSA-102, CNG-HSA-104, CNG-HSA-109, CNG-HSA-116, or CNG-HSA-117 binds protein A with a K_{D} in the range of 1-2.5 nM or 1-2 nM, as measured by SPR when the antigen-binding site is present as a monomer.

Melting temperature represents the thermostability of the antigen-binding site and can be measured by differential scanning fluorimetry, for example, as described in Durowoju et al. (2017) J. Vis. Exp. (121): 55262. The thermostability of an antibody or fragment thereof may be enhanced by grafting CDRs onto stable frameworks, introducing non-canonical disulfide bonds, and other mutagenesis, as described in McConnell et al. (2014) MAbs, 6(5): 1274-82; and Goldman et al. (2017) Front. Immunol., 8: 865. In certain embodiments, the antigen-binding site derived from CNG-HSA-101, CNG-HSA-102, CNG-HSA-103, CNG-HSA-104, CNG-HSA-105, CNG-HSA-106, CNG-HSA-108, CNG-HSA-109, CNG-HSA-113, CNG-HSA-116, CNG-HSA-117, or CNG-HSA-120 has a melting temperature greater than or equal to 60° C., as measured by differential scanning fluorimetry. In certain embodiments, the antigen-binding site derived from CNG-HSA-101, CNG-HSA-102, CNG-HSA-103, CNG-HSA-106, or CNG-HSA-120 has a melting temperature greater than or equal to 65° C., as measured by differential scanning fluorimetry.

The present disclosure also provides an antigen-binding site that competes for binding serum (e.g., human serum albumin) and/or competes for binding protein A with an antibody or antigen-binding site comprising the VH sequence provided in Table 1.

### II. Multi-Specific Binding Proteins

In one aspect, the present disclosure provides a multi-specific binding protein that comprises a domain that binds a target molecule (e.g., a target protein expressed on a target cell) and an antigen-binding site disclosed in section I above titled “Anti-Serum Albumin Antibodies.” In certain embodiments, the multi-specific binding protein comprises a first domain (e.g., a first antigen-binding site) that binds a first target protein expressed on a target cell e.g.; and/or a second domain (e.g., a second antigen-binding site) that binds a second target protein expressed on an immune effector cell e.g.; and a third domain (e.g., a third antigen-binding site) that binds serum albumin (e.g., HSA), wherein the third domain comprises an antigen-binding site disclosed in section I above titled “Anti-Serum Albumin Antibodies.” The first target can be a molecule (e.g., protein) expressed on a target cell that is desirable to clear (e.g., a cancer cell or a cell in a tumor microenvironment), such as CD19, HER2, BCMA, CD33, or EGFR. The second target can be a molecule (e.g., protein) expressed on an immune effector cell (e.g., T cell or NK cell), such as CD3 (e.g., CD3ε (epsilon), CD3δ (delta), and/or CD3γ (gamma)), 4-1BB, NKG2D, or NKp30. It is contemplated that multi-specific binding proteins binding such first and second targets can facilitate clearance of the cells that express the first target.

In certain embodiments, the first, second, and third domains comprise a first antigen-binding site, a second antigen-binding site, and a third antigen-binding site, respectively. Each of the antigen-binding sites of the multi-specific binding protein can take various forms, such as single-chain variable fragment (scFv), Fab fragment, or single domain antibody (sdAb). In certain embodiments, the first antigen-binding site comprises an scFv. In certain embodiments, the second antigen-binding site comprises an scFv. In certain embodiments, the third antigen-binding site comprises an sdAb.

Alternatively, it is also contemplated that one or more of the binding domains may not comprise an antigen-binding site. For example, U.S. Patent Application Publication No. US20130316952A1 discloses a polypeptide that binds serum albumin having the amino acid sequence of LKEAKEKAIEELKKAGITSDYYFDLINKAKTVEGVNALKDEILKA (SEQ ID NO: 282). Additional exemplary polypeptides that bind HSA are described in Dennis et al. (2002) J. Biol. Chem., 277: 35035-43; Jacobs et al. (2015) Protein Eng. Des. Sel., 28: 385-93; and Zorzi et al. (2017) Nat. Commun., 8: 16092.

In certain embodiments, the multi-specific binding protein further comprises an antibody Fc region. The presence of an Fc region may increase the serum half-life of the multi-specific binding protein. Depending on the specific Fc subtype and variant used, the Fc region may also alter the activity (e.g., cytotoxic activity) of the multi-specific binding protein.

In other embodiments, the multi-specific binding protein does not comprise an antibody Fc region. The absence of Fc contributes to a smaller size of the multi-specific binding protein, which can exhibit improved tissue penetration and pharmacokinetic properties. In certain embodiments, the multi-specific binding proteins consists of or consists essentially of the first, second, and third antigen-binding sites and the linkers between them. In certain embodiments, the multi-specific binding proteins consists essentially of the first, second, and third antigen-binding sites.

In certain embodiments, the multi-specific binding protein binds the first target protein, the second target protein, and/or serum albumin monovalently. The exclusion of additional binding domains reduces the risk of non-specific immune cell activation and decreases the size of the multi-specific binding protein.

### A. First Antigen-Binding Site

In certain embodiments, the first antigen-binding site of the multi-specific binding protein binds CD19 (e.g., human CD19). In certain embodiments, the first antigen-binding site of the multi-specific binding protein binds FLT3 (e.g., human FLT3).

The first antigen-binding site that binds CD19 can be derived from, for example, MT-103 (a single-chain bispecific CD19/CD3 antibody; see, Hoffman et al. (2005) Int. J. Cancer, 115: 98-104; Schlereth et al. (2006) Cancer Immunol. Immunother. 55: 503-14), a CD19/CD16 diabody (see, Schlenzka et al. (2004) Anti-cancer Drugs 15: 915-19; Kipriyanov et al. (2002) J. Immunol. 169: 137-44), BU12-saporin (see, Flavell et al. (1995) Br. J. Cancer 72: 1373-79), and anti-CD19-idarubicin (see, Rowland et al. (1993) Cancer Immunol. Immunother. 55: 503-14). Additional exemplary antigen-binding sites that bind CD19, from which the instant first antigen-binding site may be derived, are disclosed in U.S. Patent Application Publication Nos. US20170174786A1, US20090042291A1, US20160046730A1, US20070154473A1, US20090142349A1, US20180142018A1, US20090136526A1, US20060257398A1, and US20180230225A1, and PCT Publication No. WO2019057100A1. For example, in certain embodiments, the first antigen-binding site that binds CD19 is derived from an antibody listed in Table 2.

TABLE 2
Sequences of Exemplary Antibodies That Bind CD19

| Antibody | VH and HCDRs | VL and LCDRs |
|---|---|---|
| CNG- | QVQLVQSGAEVKKPGASVKVSCKASG | DIVMTQTPLSLSVTPGQPASISCKSS |
| CD19-1 | YTFTDYIMHWVRQAPGQGLEWMGYIN | QSLETSTGTTYLNWYLQKPGQSPQ |
| | PYNDGSKYTEKFQGRVTMTSDTSISTA | LLIYRVSKRFSGVPDRFSGSGSGTD |
| | YMELSRLRSDDTAVYYCARGTYYYGP | FTLKISRVEAEDVGVYYCLQLLEDP |
| | QLFDYWGQGTTVTVSS (SEQ ID NO: | YTFGQGTKLEIK (SEQ ID NO: 13) |
| | 20) | LCDR1: KSSQSLETSTGTTYLN (SEQ |
| | HCDR1*: YTFTDYIMH (SEQ ID NO: 22) | ID NO: 18) |
| | HCDR1: DYIMH (SEQ ID NO: 4) | LCDR2: RVSKRFS (SEQ ID NO: 19) |
| | HCDR2: YINPYNDGSKYTEKFQG (SEQ | LCDR3: LQLLEDPYT (SEQ ID NO: |
| | ID NO: 23) | 10) |
| | HCDR3*: ARGTYYYGPQLFDY (SEQ ID | |
| | NO: 16) | |
| | HCDR3: GTYYYGPQLFDY (SEQ ID NO: | |
| | 17) | |
| CNG- | QVQLQESGPGLVKPSQTLSLTCTVSGGS | EIVLTQSPATLSLSPGERATLSCSAS |
| CD19-7 | ISTSGMGVGWIRQHPGKGLEWIGHIWW | SSVSYMHWYQQKPGQAPRLLIYDT |
| | DDDKRYNPALKSRVTISVDTSKNQFSL | SKLASGIPARFSGSGSGTDFTLTISSL |
| | KLSSVTAADTAVYYCARMELWSYYFD | EPEDFAVYYCFQGSVYPFTFGQGT |
| | YWGQGTLVTVSS (SEQ ID NO: 90) | KLEIK (SEQ ID NO: 74) |
| | HCDR1*: GSISTSGMGVG (SEQ ID NO: | LCDR1: SASSSVSYMH (SEQ ID NO: |
| | 92) | 80) |
| | HCDR1: TSGMGVG (SEQ ID NO: 93) | LCDR2: DTSKLAS (SEQ ID NO: 70) |
| | HCDR2: HIWWDDDKRYNPALKS (SEQ | LCDR3: FQGSVYPFT (SEQ ID NO: |
| | ID NO: 87) | 71) |
| | HCDR3*: ARMELWSYYFDY (SEQ ID | |
| | NO: 67) | |
| | HCDR3: MELWSYYFDY (SEQ ID NO: | |
| | 68) | |
| Xencor | EVQLVESGGGLVKPGGSLKLSCAASGY | DIVMTQSPATLSLSPGERATLSCRSS |
| mAb | TFTSYVMHWVRQAPGKGLEWIGYINP | KSLQNVNGNTYLYWFQQKPGQSP |
| | YNDGTKYNEKFQGRVTISSDKSISTAY | QLLIYRMSNLNSGVPDRFSGSGSGT |
| | MELSSLRSEDTAMYYCARGTYYYGTR | EFTLTISSLEPEDFAVYYCMQHLEY |
| | VFDYWGQGTLVTVSS (SEQ ID NO: 197) | PITFGAGTKLEIK (SEQ ID NO: 198) |
| | HCDR1: SYVMH (SEQ ID NO: 199) | LCDR1: RSSKSLQNVNGNTYLY |
| | HCDR2: WIGYINPYNDGTKY (SEQ ID | (SEQ ID NO: 202) |
| | NO: 200) | LCDR2: RMSNLNS (SEQ ID NO: 203) |
| | HCDR3: GTYYYGTRVFDY (SEQ ID NO: | LCDR3: MQHLEYPIT (SEQ ID NO: |
| | 201) | 204) |
| Abbvie | QVQLQQSGAELVRPGSSVKISCKASGY | DILLTQTPASLAVSLGQRATISCKAS |
| mAb | AFSSYWMNWVKQRPGQGLEWIGQIWP | QSVDYDGDSYLNWYQQIPGQPPKL |
| | GDGDTNYNGKFKGKATLTADESSSTA | LIYDASNLVSGIPPRFSGSGSGTDFT |
| | YMQLSSLASEDSAVYFCARRETTTVGR | LNIHPVEKVDAATYHCQQSTEDPW |
| | YYYAMDYWGQGTSVTVSS (SEQ ID | TFGGGTKLEIK (SEQ ID NO: 206) |
| | NO: 205) | LCDR1: KASQSVDYDGDSYLN |
| | HCDR1: SYWMN (SEQ ID NO: 207) | (SEQ ID NO: 210) |
| | HCDR2: QIWPGDGDTNYNGKFKG (SEQ | LCDR2: DASNLVS (SEQ ID NO: 211) |
| | ID NO: 208) | LCDR3: QQSTEDPWT (SEQ ID NO: |
| | HCDR3: RETTTVGRYYYAMDY (SEQ ID | 212) |
| | NO: 209) | |
| Immunomedics | QVQLQQSGAEVKKPGSSVKVSCKASG | DIQLTQSPSSLSASVGDRVTITCKAS |
| mAb | YAFSSYWMNWVRQRPGQGLEWIGQIW | QSVDYDGDSYLNWYQQIPGKAPKL |
| | PGDGDTNYNGKFKGRATITADESTNTA | LIYDASNLVSGIPPRFSGSGSGTDYT |
| | YMELSSLRSEDTAFYSCARRETTTVGR | FTISSLQPEDIATYHCQQSTEDPWTF |
| | YYYAMDYWGQGTTVTVSS (SEQ ID | GGGTKLQIKR (SEQ ID NO: 214) |
| | NO: 213) | LCDR1: KASQSVDYDGDSYLN |
| | HCDR1: SYWMN (SEQ ID NO: 29) | (SEQ ID NO: 217) |
| | HCDR2: QIWPGDGDTNYNGKFKG (SEQ | LCDR2: DASNLVS (SEQ ID NO: 218) |
| | ID NO: 215) | LCDR3: QQSTEDPWT (SEQ ID NO: |
| | HCDR3: RETTTVGRYYYAMDY (SEQ ID | 219) |
| | NO: 216) | |
| Merck | QVQLEQPGAEVVKPGASVKVSCKTSG | QIVLTQSPATLSASPGEKATMTCSA |
| mAb | YTFTSNWMHWVKQTPGKGLEWIGEID | SSGVNYMHWYQQKPGTSPKRWIY |
| | PSDSYTNYNQKFDGKAKLTVDKSSSTA | DTDKTASGVPARFSGSGSGTSYSLT |
| | YMEVSDLTAEDSATYYCARGSNPYYY | ISSMEAEDAATYYCHQRGSYTFGG |
| | AMDYWGQGTSVTVSS (SEQ ID NO: | GTKLEIK (SEQ ID NO: 221) |
| | 220) | LCDR1: SASSGVNYMH (SEQ ID NO: |
| | HCDR1: SNWMH (SEQ ID NO: 222) | 225) |
| | HCDR2: EIDPSDSYTN (SEQ ID NO: 223) | LCDR2: DTDKTAS (SEQ ID NO: 226) |
| | HCDR3: GSNPYYYAMDY (SEQ ID NO: | LCDR3: HQRGSYT (SEQ ID NO: 227) |
| | 224) | |
| Medarex | EVQLVQSGAEVKKPGESLKISCKGSGY | AIQLTQSPSSLSASVGDRVTITCRAS |
| mAb | SFSSSWIGWVRQMPGKGLEWMGIIYPD | QGISSALAWYQQKPGKAPKLLIYD |
| 21D4a | DSDTRYSPSFQGQVTISADKSIRTAYLQ | ASSLESGVPSRFSGSGSGTDFTLTIS |
| | WSSLKASDTAMYYCARHVTMIWGVII | SLQPEDFATYYCQQFNSYPFTFGPG |
| | DFWGQGTLVTVSS (SEQ ID NO: 228) | TKVDIK (SEQ ID NO: 229) |
| | HCDR1: SSWIG (SEQ ID NO: 230) | LCDR1: RASQGISSALA (SEQ ID NO: |
| | HCDR2: IIYPDDSDTRYSPSFQG (SEQ ID | 233) |
| | NO: 231) | LCDR2: DASSLES (SEQ ID NO: 234) |
| | HCDR3: HVTMIWGVIIDF (SEQ ID NO: | LCDR3: QQFNSYPFT (SEQ ID NO: |
| | 232) | 235) |
| Medarex | EVQLVQSGAEVKKPGESLKISCKGSGY | AIQLTQSPSSLSASVGDRVTITCRAS |
| mAb | SFSSSWIGWVRQMPGKGLEWMGIIYPD | QGISSALAWYQQKPGKAPKLLIYD |
| 21D4 | DSDTRYSPSFQGQVTISADKSIRTAYLQ | ASSLESGVPSRFSGSGSGTDFTLTIS |
| | WSSLKASDTAMYYCARHVTMIWGVII | SLQPEDFATYYCQQFNSYPYTFGQ |
| | DFWGQGTLVTVSS (SEQ ID NO: 236) | GTKLEIK (SEQ ID NO: 237) |
| | HCDR1: SSWIG (SEQ ID NO: 238) | LCDR1: RASQGISSALA (SEQ ID NO: |
| | HCDR2: IIYPDDSDTRYSPSFQG (SEQ ID | 241) |
| | NO: 239) | LCDR2: DASSLES (SEQ ID NO: 242) |
| | HCDR3: HVTMIWGVIIDF (SEQ ID NO: | LCDR3: QQFNSYPYT (SEQ ID NO: |
| | 240) | 243) |
| Medarex | QVQLVQSGAEVKKPGSSVKVSCKDSG | EIVLTQSPGTLSLSPGERATLSCRAS |
| mAb | GTFSSYAISWVRQAPGQGLEWMGGIIPI | QSVSSSYLAWYQQKPGQAPRLLIY |
| 47G4 | FGTTNYAQQFQGRVTITADESTSTAYM | GASSRATGIPDRFSGSGSGTDFTLTI |
| | ELSSLRSEDTAVYYCAREAVAADWLDP | SRLEPEDFAVYYCQQYGSSRFTFGP |
| | WGQGTLVTVSS (SEQ ID NO: 244) | GTKVDIK (SEQ ID NO: 245) |
| | HCDR1: SYAIS (SEQ ID NO: 246) | LCDR1: RASQSVSSSYLA (SEQ ID |
| | HCDR2: GIIPIFGTTNYAQQFQG (SEQ ID | NO: 249) |
| | NO: 247) | LCDR2: GASSRAT (SEQ ID NO: 250) |
| | HCDR3: EAVAADWLDP (SEQ ID NO: | LCDR3: QQYGSSRFT (SEQ ID NO: |
| | 248) | 251) |
| Medarex | EVQLVQSGAEVKKPGESLKISCKGSGY | AIQLTQSPSSLSASVGDRVTITCRAS |
| mAb 27F3 | SFTSYWIAWVRQMPGKGLEWMGIIYPG | QGISSALAWYQQKPGKAPKLLIYD |
| | DSDTRYSPSFQGQVTISADKSISTAYLQ | ASSLESGVPSRFSGSGSGTDFTLTIS |
| | WSSLKASDTAMYYCARQGYSSGWDSY | SLQPEDFATYYCQQFNSYPYTFGQ |
| | YGMGVWGQGTTVTVSS (SEQ ID NO: | GTKLEIK (SEQ ID NO: 253) |
| | 252) | LCDR1: RASQGISSALA (SEQ ID NO: |
| | HCDR1: SYWIA (SEQ ID NO: 254) | 257) |
| | HCDR2: IIYPGDSDTRYSPSFQG (SEQ ID | LCDR2: DASSLES (SEQ ID NO: 258) |
| | NO: 255) | LCDR3: QQFNSYPYT (SEQ ID NO: |
| | HCDR3: QGYSSGWDSYYGMGV (SEQ | 259) |
| | ID NO: 256) | |
| Medarex | QVQLVQSGAEVKKPGSSVKVSCKASG | DIQMTQSPSSLSASVGDRVTITCRA |
| mAb | GTFSSYTINWVRQAPGQGLEWMGGIIPI | SQGISSWLAWYQQKPEKAPKSLIY |
| 3C10 | FGIPNYAQKFQGRVTITADESTNTAYM | AASSLQSGVPSRFSGSGSGTDFTLTI |
| | ELSSLRAEDTAVYYCARASGGSADYSY | SSLQPEDFATYYCQQYKRYPYTFG |
| | GMDVWGQGTAVTVSS (SEQ ID NO: | QGTKLEIK (SEQ ID NO: 261) |
| | 260) | LCDR1: RASQGISSWLA (SEQ ID |
| | HCDR1: SYTIN (SEQ ID NO: 262) | NO: 265) |
| | HCDR2: GIIPIFGIPNYAQKFQG (SEQ ID | LCDR2: AASSLQS (SEQ ID NO: 266) |
| | NO: 263) | LCDR3: QQYKRYPYT (SEQ ID NO: |
| | HCDR3: ASGGSADYSYGMDV (SEQ ID | 267) |
| | NO: 264) | |
| Medarex | EVQLVQSGAEVKKPGESLNISCKGSGY | AIQLTQSPSSLSASVGDRVTITCRAS |
| mAb 5G7 | SFTSYWIGWVRQMPGKGLEWMGIIYPG | QGISSALAWYQQKPGKAPKLLIYD |
| | DSDTRYSPSFQGQVTISADKSINTAYLQ | ASSLESGVPSRFSGSGSGTDFTLTIS |
| | WSSLKASDTAMYYCARGVSMIWGVIM | SLQPEDFATYYCQQFNSYPWTFGQ |
| | DVWGQGTTVTVSS (SEQ ID NO: 268) | GTKVEIK (SEQ ID NO: 269) |
| | HCDR1: SYWIG (SEQ ID NO: 270) | LCDR1: RASQGISSALA (SEQ ID NO: |
| | HCDR2: IIYPGDSDTRYSPSFQG (SEQ ID | 273) |
| | NO: 271) | LCDR2: DASSLES (SEQ ID NO: 274) |
| | HCDR3: GVSMIWGVIMDV (SEQ ID NO: | LCDR3: QQFNSYPWT (SEQ ID NO: |
| | 272) | 275) |
| Medarex | EVQLVQSGAEVKKPGESLQISCKGSGY | AIQLTQSPSSLSASVGDRVTITCRAS |
| mAb 13F1 | TFTNYWIAWVRQMPGKGLEWMGIIYP | QGISSALAWYQQKPGKAPKLLIYD |
| | GDSDTRYSPSFQGQVTISADKSISTAYL | ASSLESGVPSRFSGSGSGTDFTLTIS |
| | QWSGLKASDTAMYYCARQGYSSGWR | SLQPEDFATYYCQQFNSYPHTFGQ |
| | SYYGMGVWGQGTTVTVSS (SEQ ID | GTKLEIK (SEQ ID NO: 277) |
| | NO: 276) | LCDR1: RASQGISSALA (SEQ ID NO: |
| | HCDR1: NYWIA (SEQ ID NO: 278) | 281) |
| | HCDR2: IIYPGDSDTRYSPSFQG (SEQ ID | LCDR2: DASSLES (SEQ ID NO: 282) |
| | NO: 279) | LCDR3: QQFNSYPHT (SEQ ID NO: |
| | HCDR3: QGYSSGWRSYYGMGV (SEQ | 283) |
| | ID NO: 280) | |
| Medarex | EVQLVQSGAEVKKPGESLQISCKGSGY | AIQLTQSPSSLSASVGDRVTITCRAS |
| mAb 46E8 | TFTNYWIAWVRQMPGKGLEWMGIIYP | QGISSALAWYQQKPGKAPKLLIYD |
| | GDSDTRYSPSFQGQVTISADKSISTAYL | ASSLESGVPSRFSGSGSGTDFTLTIS |
| | QWSGLKASDTAMYYCARQGYSSGWR | SLQPEDFATYYCQQFNSYPHTFGQ |
| | SYYGMGVWGQGTTVTVSS (SEQ ID | GTKLEIK (SEQ ID NO: 285) |
| | NO: 284) | LCDR1: RASQGISSALA (SEQ ID NO: |
| | HCDR1: NYWIA (SEQ ID NO: 286) | 289) |
| | HCDR2: IIYPGDSDTRYSPSFQG (SEQ ID | LCDR2: DASSLES (SEQ ID NO: 290) |
| | NO: 287) | LCDR3: QQFNSYPHT (SEQ ID NO: |
| | HCDR3: QGYSSGWRSYYGMGV (SEQ | 291) |
| | ID NO: 288) | |
| Novimmune | EVQLVQSGAEVKKPGESLKISCKGSGY | DIQMTQSPSSLSASVGDRVTITCRA |
| mAb | SFTSYWIGWVRQMPGKGLEWMGIIYPG | SQSISSYLNWYQQKPGKAPKLLIYA |
| | DSDTRYSPSFQGQVTISADKSISTAYLQ | ASSLQSGVPSRFSGSGSGTDFTLTIS |
| | WSSLKASDTAMYYCARGVSGIYNLHG | SLQPEDFATYYCQQGRFGSPFTFGQ |
| | FDIWGQGTLVTVSS (SEQ ID NO: 315) | GTKVEIK (SEQ ID NO: 316) |
| | HCDR1: GYSFTSYW (SEQ ID NO: 317) | LCDR1: QSISSY (SEQ ID NO: 320) |
| | HCDR2: IYPGDSDT (SEQ ID NO: 318) | LCDR2: AAS (SEQ ID NO: 321) |
| | HCDR3: ARGVSGIYNLHGFDI (SEQ ID | LCDR3: QQGRFGSPFT (SEQ ID NO: |
| | NO: 319) | 322) |
| Eureka | QVQLVETGGGLVQPGGSLRLSCAASGF | QTVVTQEPSVSAAPGQKVTISCSGS |
| mAb-1 | TFSSYAMSWVRQAPGKGLEWVSAISGS | SSNIGNNYVSWYQQLPGTAPKLLIY |
| | GGSTYYADSVKGRFTISRDNSKNTLYL | DNNKRPSGIPDRFSGSKSGTSATLGI |
| | QMNSLRAEDTAVYYCARYYYSRLDY | TGLQTGDEADYYCGTWDSSLSAGV |
| | WGQGTLVTVSS (SEQ ID NO: 323) | FGTGTKLTVLGSR (SEQ ID NO: 324) |
| Eureka | QVQLVESGGGLVQPGGSLRLSCAASGF | QSVLTQPPSVSAAPGQKVTISCSGSS |
| mAb-2 | TFSSYAMSWVRQAPGKGLEWVSGISAS | SNIGNNYVSWYRQLPGTAPKLLIYE |
| | GGSTYYADSVKGRFTISRDNSKNTLYL | NNKRPSGIPDRFSGSKSGTSATLGIT |
| | QMNSLRAEDTAVYYCARYYLSQIDSW | GLQTGDEADYYCGTWDSSLRAGV |
| | GQGTLVTVSS (SEQ ID NO: 325) | FGTGTKVTVL (SEQ ID NO: 326) |
| Eureka | EVQLVQSGAEVKKPGATVKISCKVSGY | QSVLTQPPSASGTPGQRVTISCSGSS |
| mAb-3 | TFTDYYMHWVQQAPGKGLEWMGLVD | SNIGSNTVNWYQQLPGTAPKLLIYS |
| | PEDGETIYAEKFQGRVTITADTSTDTAY | NNQRPSGVPDRFSGSKSGTSASLAI |
| | MELSSLRSEDTAVYYCATGIYSRPLGY | SGLQSEDEADYYCAAWDDSLNGH |
| | WGQGTLVTVSS (SEQ ID NO: 327) | VVFGGGTKLTVL (SEQ ID NO: 328) |
| Eureka | EVQLVETGGGLVQPGGSLRLSCAASGF | SYVLTQPPSASGTPGQRVTISCSGSS |
| mAb-4 | TFSSYAMSWVRQAPGKGLEWVSAISGS | SNIGSHTVNWYQQLPGTAPKLLIYS |
| | GGSTYYADSVKGRFTISRDNSKNTLYL | NNQRPSGVPDRFSGSKSGTSASLAI |
| | QMNSLRAEDTAVYYCARSDGKHFWQ | SGLQSEDEADYYCAAWDDSLNGY |
| | QYDAWGQGTLVTVSS (SEQ ID NO: | VFGTGTKVTVL (SEQ ID NO: 330) |
| | 329) | |
| Eureka | EVQLVESGGGLVQPGGSLRLSCAASGF | DIQLTQSPSSLSAYVGDRVTITCRAS |
| mAb-5 | TVS SNYMS WVRQAPGKGLEWVS AISG | QGITNSLAWYQQKPGKAPKLLLHA |
| | SGGSTYYADSVKGRFTISRDNSKNTLY | ASRLESGVPSRFSGSGFGTDFTLTIS |
| | LQMNSLRAEDTAVYYCARMNIDYWG | SLQPEDFAVYYCQHYLGTPYSFGQ |
| | QGTLVTVSS (SEQ ID NO: 331) | GTKVEIK (SEQ ID NO: 332) |
| Eureka | EVQLVQSGAEVKRPGESLTISCKGSEYS | EIVLTQSPSSLSASVGDRVTISCRAS |
| mAb-6 | FASYWITWVRQMPGKGLEWMGRIDPS | QSVSRFLNWYQQKPGKAPKLLIYG |
| | DSYTNYSPSFQGHVTISADKSISTAYLQ | VSTLERGVPSRFSGSGSGTDFTLTIS |
| | WS SLKASDTAIYYCARPFQYDYGGYSD | SLQPEDFATYYCQESYIIPLTFGGGT |
| | AFDIWGQGTMVTVSS (SEQ ID NO: 333) | KLEIK (SEQ ID NO: 334) |
| Eureka | QMQLVQSGAEVKKAGSSVKVSCETSG | EIVMTQSPLSLSVTPGEPASISCRSS |
| mAb-7 | GTFSSSSVNWVRQAPGQGLEWMGGIIPI | QSLLDSNGFNSLDWYLQKPGQSPQ |
| | VGTPNYAQKFQDRVTITAVESTFTAYM | LLIHLGSDRASGVPDRFSGSGSGTD |
| | ELSGLRSEDTAVYYCARGGYRDYMDV | FTLKISRVEAEDVGIYYCMQSLQIPT |
| | WGRGTTVTVSS (SEQ ID NO: 335) | FGQGTKVEIK (SEQ ID NO: 336) |
| Eureka | EVQLVESGGGLIQPGGSLRLSCAASGFT | SYELTQPPSASGTPGQRVTISCSGSS |
| mAb-8 | VSSNYMSWVRQAPGKGLEWVSVIYSG | SNIGSNYVYWYQQLPGTAPKLLIYR |
| | GSTYYADSVKGRFTISRDNSKNTLYLQ | NNQRPSGVPDRFSGSKSGTSASLAI |
| | MNSLRAEDTAVYYCARGGFGAEFDYW | SGLRSEDEADYYCAAWDDSLSGYV |
| | GQGTLVTVSS (SEQ ID NO: 337) | FGTGTKVTVL (SEQ ID NO: 338) |
| Eureka | EVQLVESGGGLIQPGGSLRLSCAASGFT | SYVLTQPPSVSVSPGQTASITCSGD |
| mAb-9 | VSSNYMSWVRQAPGKGLEWVSVIYSG | KLGDKYASWYQQKPGQSPVLVIYQ |
| | GSTYYADSVKGRFTISRDNSKNTLYLQ | DNKRPSGIPERFSGSNSGNTATLTIS |
| | MNSLRAEDTAVYYCARGGISDDYYGS | GTQAMDEADYYCQAWDSSTEDVF |
| | GSYDNWGQGTLVTVSS (SEQ ID NO: | GPGTKVTVL (SEQ ID NO: 340) |
| | 339) | |
| Eureka | EVQLVESGGGLVQPGGSLRLSCAASGF | DIQLTQSPSSLSASVGDRVTITCRAS |
| mAb-10 | TVSSNYMSWVRQAPGKGLEWVSVIYS | QSISSYLNWYQQKPGKAPKLLIYAA |
| | GGSTYYADSVKGRFTISRDNSKNTLYL | SSLQSGVPSRFSGSGSGTDFTLTISS |
| | QMNSLRAEDTAVYYCARERGMGYAFD | LQPEDFATYYCQQSYSTPFTFGGGT |
| | IWGQGTMVTVSS (SEQ ID NO: 341) | KVEIK (SEQ ID NO: 342) |
| Eureka | QLQLQESGPGLVKPSETLSLTCSVSGVS | DIQMTQSPSSLSASVGDRVTITCRA |
| mAb-11 | MSENYWSWIRQPPGKRLEWIGCAHYT | SQGIGSYLAWYQQKPGKAPKLLIYP |
| | GDTHYNPSLKGRVTISLDTSMNQFSLRL | ASTLQSGVPSRFSGSGSGTEFTLTIS |
| | NSVTAADTAVYYCASYHPFNYWGQGT | SLQPEDFATYYCQQLNSLFGQGTRL |
| | LVTVSS (SEQ ID NO: 343) | EIK (SEQ ID NO: 344) |
| Eureka | EVQLVQSGAEVRRPGATVKISCKVSGY | QAVLTQPPSASGTPGQRVTISCSGSS |
| mAb-12 | TFNDFYLHWVRQAPGKGLEWMGRIDP | SNIGTKTVNWYQVLPGTAPKLLIYS |
| | EDGKTRYAEKFQGRLTITADTSTDTLY | NYRRPSGVPDRFSGSKSGTSASLAIS |
| | MQLGGLTSDDTAVYYCTTDWGYSSSL | GLQSDDEADYYCALWDDSLDGYV |
| | REEDIWYDCWGQGTLVTVSS (SEQ ID | FGTGTKVTVL (SEQ ID NO: 346) |
| | NO: 345) | |
| Eureka | EVQLVQSGAEVKKPGSSVKVSCKASGG | SYELTQPPSVSVAPGKTARITCGGN |
| mAb-13 | TFSSYAISWVRQAPGQGLEWMGGIIPIF | NIGSKSVHWYQQKPGQAPVLVIYY |
| | GTANYAQKFQGRVTITADESTSTAYME | DSDRPSGIPERFSGSNSGNTATLTIS |
| | LSSLRSEDTAVYYCARDYGYGD YGD A | RVEAGDEADYYCQVWDSSSDHYV |
| | FDIWGQGTMVTVSS (SEQ ID NO: 347) | FGTGTKVTVL (SEQ ID NO: 348) |
| Eureka | EVQLVQSGAEVKKPGESLKISCKGSGY | SYVLTQPPSVSVAPGKTARITCGGN |
| mAb-14 | SFTSYWIGWVRQMPGKGLEWMGIIYPG | NIGSKSVHWYQQRPGQAPVLVVYD |
| | DSDTRYSPSFQGQVTISADKSISTAYLQ | DSDRPSGIPERFSGSNSGNTATLTIS |
| | WSSLKASDTAMYYCARVVGTIYSMQY | RVEAGDEADYSCQVWDSSSDHYV |
| | DVWGQGTLVTVSS (SEQ ID NO: 349) | FGPGTKVTVL (SEQ ID NO: 350) |
| Eureka | EVQLVQSGAEVKKPGESLKISCKGSGY | LPVLTQPPSVSVAPGKTARITCGGN |
| mAb-15 | SFTSYWIGWVRQMPGKGLEWMGIIYPG | NIGSKSVHWYQQKPGQAPVLVVY |
| | DSDTRYSPSFQGQVTISADKSISTAYLQ | DDSDRPSGIPERFSGSNSGNTATLTI |
| | WSSLKASDTAMYYCARQVWGWQGG | SRVEAGDEADYYCQVWDSSSDYV |
| | MYPRSNWWYNMDSWGQGTLVTVSS | VFGGGTKLTVL (SEQ ID NO: 352) |
| | (SEQ ID NO: 351) | |
| Eureka | EVQLVQSGAEVKKPGESLKISCKGSGY | QAVLTQPPSVSEAPRQRVTISCSGSS |
| mAb-16 | SFTSYWIGWVRQMPGKGLEWMGIIYPG | SNVGNNAVNWYQQVPGKAPKLLI |
| | DSDTRYSPSFQGQVTISADKSISTAYLQ | YYDDLLSSGVSDRFSGSKSGTSASL |
| | WSSLKASDTAMYYCARWSSTWDSMY | AISGLQSEDEADYYCAAWDDSLNG |
| | MDYWGQGTLVTVSS (SEQ ID NO: 353) | PVFGGGTKLTVL (SEQ ID NO: 354) |
| Eureka | EVQLVQSGAEVKKPGESLRISCKGSGY | QPVLTQPPSVSVAPGKTARITCGGN |
| mAb-17 | SFTSYWIGWVRQMPGKGLEWMGIIYPG | NIGSESVHWYQQKPGQAPMVVIYY |
| | DSDTRYSPSFQGQVTISADKSISTAYLQ | DSNRPSGIPERFSGSNSGNTATLTVS |
| | WSSLKASDTAMYYCARVTYSMDSYYF | RVEAEDEADYYCQVWNSSSDHRG |
| | DSWGQGTLVTVSS (SEQ ID NO: 355) | VFGGGTKLTV (SEQ ID NO: 356) |
| WuXi | EVQLQQSGPELVKPGASVKMSCKASG | DAVMTQTPLSLPVSLGDQASISCRS |
| WBP7011- | YTFTNYVIHWVKQKPGQGLEWIGYFNP | SQSLENSNGNTYLNWYLQKPGQSP |
| 4.34.11 | YNDGTEYNEKFKAKATLTSDKSSSTAY | QLLIYRVSNRFSGVLDRFSGSGSGT |
| | MELSSLTSEDSAVYYCAKGPYYYGSSP | DFTLKISRVEAEDLGVYFCLQVTHV |
| | FDYWGQGTTLTVSS (SEQ ID NO: 357) | PYTFGGGTKLEIK (SEQ ID NO: 358) |
| | HCDR1: GYTFTNYVIH (SEQ ID NO: 359) | LCDR1: RSSQSLENSNGNTYLN |
| | HCDR2: YFNPYNDGTEYNEKFKA (SEQ | (SEQ ID NO: 362) |
| | ID NO: 360) | LCDR2: RVSNRFS (SEQ ID NO: 363) |
| | HCDR3: GPYYYGSSPFDY (SEQ ID NO: | LCDR3: RVSNRFS (SEQ ID NO: 364) |
| | 361) | |
| WuXi | QVQLQQSGAELVRPGSSVKISCKASGY | DIQMTQTTSSLSASLGDRVTISCRAS |
| WBP7011- | AFSTYWMNWVKQRPGQGLEWIGQIYP | QDISNYLNWYQQKPDGTVKLLIYY |
| 4.87.6 | GDDDTKYNGKFKGKASLTADKSSSTA | TSRLHSGVPARFSGSGSGTDYSLTIS |
| | YMQLISLTSEDSAVYFCARRYFRYDYW | NLEQEDIATYFCHQGNTLPLTFGAG |
| | YSDVWGAGTTVTVTS (SEQ ID NO: 365) | TKLELK (SEQ ID NO: 366) |
| | HCDR1: GYAFSTYWMN (SEQ ID NO: | LCDR1: RASQDISNYLN (SEQ ID |
| | 367) | NO: 370) |
| | HCDR2: QIYPGDDDTKYNGKFKG (SEQ | LCDR2: YTSRLHS (SEQ ID NO: 371) |
| | ID NO: 368) | LCDR3: HQGNTLPLT (SEQ ID NO: |
| | HCDR3: RYFRYDYWYSDV (SEQ ID NO: | 372) |
| | 369) | |
| WuXi | EIQLQQSGPELVKPGASVKVSCKASGY | QIVLTQSPAIMSASLGEEITLTCSAS |
| WBP7011_ | AFTSYNMYWVKQSHGKSLEWIGYIDP | STVNYMHWYQQKSGTSPKLLIYST |
| 4.155.8 | YNGDTTYNQKFKGKATLTVDKSSSTA | SNLASGVPSRFSGSGSGTFYSLTIRS |
| | YMHLNSLTSEDSAVYYCLTTAYAMDY | VEAEDAADYYCHQWSSYPYTFGG |
| | WGQGTSVTVSS (SEQ ID NO: 373) | GTKLEIK (SEQ ID NO: 374) |
| | HCDR1: GYAFTSYNMY (SEQ ID NO: | LCDR1: SASSTVNYMH (SEQ ID NO: |
| | 375) | 378) |
| | HCDR2: YIDPYNGDTTYNQKFKG (SEQ | LCDR2: STSNLAS (SEQ ID NO: 379) |
| | ID NO: 376) | LCDR3: HQWSSYPYT (SEQ ID NO: |
| | HCDR3: TAYAMDY (SEQ ID NO: 377) | 380) |
| WuXi | QVQLVQSGAEVKKPGSSVKVSCKASG | DIVMTQTPLSLPVTPGEPASISCRSS |
| WBP7011- | YTFTDYVIHWVRQAPGQGLEWMGYFN | QSLENSNHNTYINWYLQKPGQSPQ |
| 4.34.11- | PYNDGTEYNEKFKARVTITADKSTSTA | LLIYRVSKRFSGVPDRFSGSGSGTD |
| z1-m5 | YMELSSLRSEDTAVYYCARGPYYYGSS | FTLKISRVEAEDVGVYYCHQVTHV |
| | PFDYWGQGTTVTVSS (SEQ ID NO: 381) | PYTFGQGTKLEIK (SEQ ID NO: 382) |
| | HCDR1: GYTFTNYVIH (SEQ ID NO: 383) | LCDR1: RSSQSLENSNHNTYIN (SEQ |
| | HCDR2: YFNPYNDGTEYNEKFKA (SEQ | ID NO: 386) |
| | ID NO: 384) | LCDR2: RVSKRFS (SEQ ID NO: 19) |
| | HCDR3: GPYYYGSSPFDY (SEQ ID NO: | LCDR3: HQVTHVPYT (SEQ ID NO: |
| | 385) | 387) |
| WuXi | QVQLVQSGAEVKKPGASVKVSCKASG | DIQMTQSPSSLSASVGDRVTITCRA |
| WBP7011- | YAFSTYWMNWVRQAPGQGLEWMGQI | SQDISNYLNWYQQKPGKVPKLLIY |
| 4.87.6- | YPGDDDTKYSGKFKGRVTITADKSTST | YTSRLHSGVPSRFSGSGSGTDFTLTI |
| z1(N-S) | AYMELSSLRSEDTAVYYCARRYFRYD | SSLQPEDVATYYCHQGNTLPLTFG |
| | YWYSDVWGQGTTVTVSS (SEQ ID NO: | QGTKLEIK (SEQ ID NO: 389) |
| | 388) | LCDR1: RASQDISNYLN (SEQ ID |
| | HCDR1: GYAFSTYWMN (SEQ ID NO: | NO: 393) |
| | 390) | LCDR2: YTSRLHS (SEQ ID NO: 394) |
| | HCDR2: QIYPGDDDTKYSGKFKG (SEQ | LCDR3: HQGNTLPLT (SEQ ID NO: |
| | ID NO: 391) | 395) |
| | HCDR3: RYFRYDYWYSDV (SEQ ID NO: | |
| | 392) | |
| WuXi | QMQLVQSGPEVKKPGTSVKVSCKASG | DIQLTQSPSFLSASVGDRVTITCSAS |
| WBP7011_ | YAFTSYNMYWVRQARGQRLEWIGYID | STVNYMHWYQQKPGKAPKLLIYST |
| 4.155.8- | PYNADTTYNQKFKGRVTITRDMSTSTA | SNLASGVPSRFSGSGSGTEFTLTISS |
| z1-P15 | YMELS SLRSEDTAVYYCLTTAYAMD Y | LQPEDFATYYCHQWSSYPYTFGQG |
| | WGQGTLVTVSS (SEQ ID NO: 396) | TKLEIK (SEQ ID NO: 397) |
| | HCDR1: GYAFTSYNMY (SEQ ID NO: | LCDR1: SASSTVNYMH (SEQ ID NO: |
| | 398) | 401) |
| | HCDR2: YIDPYNADTTYNQKFKG (SEQ | LCDR2: STSNLAS (SEQ ID NO: 402) |
| | ID NO: 399) | LCDR3: HQWSSYPYT (SEQ ID NO: |
| | HCDR3: TAYAMDY (SEQ ID NO: 400) | 403) |
| Legend | QVKLEESGGELVQPGGPLRLSCAASGNI | N/A |
| mAb | FSINRMGWYRQAPGKQRAFVASITVRG | |
| | ITNYADSVKGRFTISVDKSKNTIYLQMN | |
| | ALKPEDTAVYYCNAVSSNRDPDYWGQ | |
| | GTQVTVSS (SEQ ID NO: 404) | |
| | HCDR1: INRMG (SEQ ID NO: 405) | |
| | HCDR2: SITVRGITNYADSVKG (SEQ ID | |
| | NO: 406) | |
| | HCDR3: VSSNRDPDY (SEQ ID NO: 407) | |

Where the VL and LCDR sequences are noted as “N/A,” the antigen-binding site is an sdAb having a VH (e.g., VHH) only.

In certain embodiments, the first antigen-binding site comprises a VH that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VH of an antibody disclosed in Table 2, and a VL that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VL of the same antibody disclosed in Table 2. In certain embodiments, the antigen-binding site comprises the HCDR1, HCDR2, HCDR3, LCDR1, LCDR2, and LCDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), IMGT (see Lefranc, (1999) The Immunologist, 7, 132-136), or any other CDR determination method known in the art, of the VH and/or VL sequences of an antibody disclosed in Table 2. In certain embodiments, the antigen-binding site comprises the HCDR1, HCDR2, HCDR3, LCDR1, LCDR2, and LCDR3 sequences of an antibody disclosed in Table 2. In certain embodiments, the antigen-binding site comprises the VH and VL sequences of an antibody disclosed in Table 2.

Such antigen-binding site may take the form of scFv. In certain embodiments, the VH is positioned C-terminal to the VL. In certain embodiments, the VH is positioned N-terminal to the VL. In certain embodiments, the VH and the VL are linked by a peptide linker, for example, a linker disclosed in subsection D below titled “Linkers.” To stabilize the scFv, the amino acid residues at position 44 of the VH and at position 100 of the VL (under Kabat numbering) can be substituted by Cys, thereby facilitating the formation of a disulfide bond between the VH and the VL. Accordingly, in certain embodiments, the VH and VL comprise Cys at positions 100 and 44, respectively.

In other embodiments, the first antigen-binding site comprises an sdAb comprising a VH comprising complementarity determining regions HCDR1, HCDR2, and HCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an sdAb antibody provided in Table 2. In certain embodiments, the VH comprises the HCDR1, HCDR2, and HCDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), IMGT (see Lefranc, (1999) The Immunologist, 7, 132-136), or any other CDR determination method known in the art, of the VH sequence of an antibody disclosed in Table 2. In certain embodiments, the VH comprises the HCDR1, HCDR2, and HCDR3 sequences of the antibody provided in Table 2. In certain embodiments, the VH comprises the amino acid sequence of the VH of an sdAb provided in Table 2.

In certain embodiments, the first antigen-binding site binds CD19 with a K_{D} lower than or equal to 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, or 10 pM. For example, in certain embodiments, the first antigen-binding site binds CD19 with a K_{D} about 10 pM-about 1 nM, about 10 pM-about 0.9 nM, about 10 pM-about 0.8 nM, about 10 pM-about 0.7 nM, about 10 pM-about 0.6 nM, about 10 pM nM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, or about 9 nM-about 10 nM.

It is understood that the binding affinity to CD19 of the first antigen-binding site alone may be different from the binding affinity of the same antigen-binding site in the context of the multi-specific binding protein disclosed herein, possibly due to the conformational restraint from the other domains. The context-dependent binding affinity is described in subsection E below titled “Binding Affinity”

In certain embodiments, the first antigen-binding site, when present in the form of an Fab, has a melting temperature of at least 60° C., at least 65° C., at least 70° C., at least 75° C., or at least 80° C. In certain embodiments, the first antigen-binding site, when present in the form of an Fab, has a melting temperature in the range of 60-85° C., 60-80° C., 60-75° C., 60-70° C., 60-65° C., 65-85° C., 65-80° C., 65-75° C., 65-70° C., 70-85° C., 70-80° C., 70-75° C., 75-85° C., 75-80° C., or 80-85° C.

### B. Second Antigen-Binding Site

In certain embodiments, the second antigen-binding site of the multi-specific binding protein binds CD3 (e.g., human CD3 and/or Macaca CD3). In certain embodiments, the second antigen-binding site binds CD3ε (epsilon). In certain embodiments, the second antigen-binding site binds CD3δ (delta). In certain embodiments, the second antigen-binding site binds CD3γ (gamma).

Earlier BiTE® constructs bind conformational epitopes of CD3 and typically are species-specific (see, PCT Publication No. WO2008119567A2). Improved BiTE® constructs, such as blinatumomab (also called AMG 103; see, PCT Publication No. WO1999054440A1) and solitomab (also called AMG 110; see, PCT Publication No. WO2005040220A1), bind context-independent epitopes at the N-terminus of CD3ε chain (e.g., amino acid residues 1-27 of human CD3ε extracellular domain) and show cross-species specificity for human, Callithrix jacchus, Saguinus Oedipus, and Saimiri sciureus CD3ε chain (see id.). These constructs do not nonspecifically activate T cells to the same degree as observed with the earlier BiTE® constructs, and are therefore believed to bear a lower risk of side effects (see, Brischwein et al. (2007) J. Immunother., 30(8): 798-807).

In certain embodiments, the second antigen-binding site of the multi-specific binding protein binds an epitope at the N-terminus of CD3ε chain. In certain embodiments, the second antigen-binding site binds an epitope localized in amino acid residues 1-27 of human CD3ε extracellular domain. This epitope or a homologous variant thereof is also present in certain non-human primates. Accordingly, in certain embodiments, the second antigen-binding site binds CD3 in different primates, for example, human, new world primates (such as Callithrix jacchus, Saguinus Oedipus, or Saimiri sciureus), old world primates (such as baboons and macaques), gibbons, and non-human homininae. Callithrix jacchus and Saguinus oedipus are new world primates belonging to the family of Callitrichidae, while Saimiri sciureus is a new world primate belonging to the family of Cebidae. In certain embodiments, the second antigen-binding site binds human CD3ε and/or Macaca CD3ε. In certain embodiments, the second antigen-binding site further binds Callithrix jacchus, Saguinus Oedipus, and/or Saimiri sciureus CD3ε.

The second antigen-binding site that binds an extracellular epitope of human and/or Macaca CD3 can be derived from, for example, muromonab-CD3 (OKT3) as described in WO2008101154; otelixizumab (TRX4) as described in WO2007145941; teplizumab (MGA031) as described in WO2013040164; visilizumab (Nuvion) as described in WO2004052397; SP34 as described in WO2015181098; X35, VIT3, or BMA030 (BW264/56) as described in WO2015006749; CLB-T3/3, CRIS7, CLB-T3.4.2, WT32, 11D8, XIII-141, XIII-46, XIII-87, 12F6, T3/RW2-8C8, T3/RW2-4B6, OKT3D, M-T301, SMC2, or F101.01 as described in WO2004106381, YTH12.5 or SPv-T3b as described in WO2004106383, F1 11-409 as described in WO2012084895, TR-66 as described in WO2013158856; UCHT-1 as described in WO2000041474; WT-31 as depicted in WO2016085889, or an antibody described in WO2008119567. For example, in certain embodiments, the second antigen-binding site that binds CD3 is derived from an antibody listed in Table 3.

TABLE 3
Sequences of Exemplary Antibodies That Bind CD3

| Antibody | VH and HCDRs | VL and LCDRs |
|---|---|---|
| CNG-CD3- | QVQLVQSGAEVKKPGASVKVSCKASG | DIVMTQSPDSLAVSLGERATINCKS |
| 1 | FNIKDYYMHWVRQAPGQRLEWMGWI | SQSLLNARTGKNYLAWYQQKPGQP |
| | DLENANTIYDAKFQGRVTITRDTSAST | PKLLIYWASTRESGVPDRFSGSGSG |
| | AYMELSSLRSEDTAVYYCARDAYGRY | TDFTLTISSLQAEDVAVYYCKQSYS |
| | FYDVWGQGTLVTVSS (SEQ ID NO: | RRTFGGGTKVEIK (SEQ ID NO: 413) |
| | 412) | LCDR1: KSSQSLLNARTGKNYLA |
| | HCDR1*: FNIKDYYMH (SEQ ID NO: | (SEQ ID NO: 419) |
| | 414) | LCDR2: WASTRES (SEQ ID NO: 420) |
| | HCDR1: DYYMH (SEQ ID NO: 415) | LCDR3: KQSYSRRT (SEQ ID NO: |
| | HCDR2: WIDLENANTIYDAKFQG (SEQ | 421) |
| | ID NO: 416) | |
| | HCDR3*: ARDAYGRYFYDV (SEQ ID | |
| | NO: 417) | |
| | HCDR3: DAYGRYFYDV (SEQ ID NO: | |
| | 418) | |
| | scFv: | |
| | DIVMTQSPDSLAVSLGERATINCKSSQSLLNARTGKNYLAWYQQKPGQPPKLLI | |
| | YWASTRESGVPDRFSGSGSGTDFTLTISSLQAEDVAVYYCKQSYSRRTFGGGT | |
| | KVEIKGGGGSGGGGSGGGGSGGGGSQVOLVOSGAEVKKPGASVKVSCKASGF | |
| | NIKDYYMHWVRQAPGQRLEWMGWIDLENANTIYDAKFQGRVTITRDTSASTA | |
| | YMELSSLRSEDTAVYYCARDAYGRYFYDVWGQGTLVTVSS (SEQ ID NO: 422) | |
| | scFv with Cys substitutions: | |
| | DIVMTQSPDSLAVSLGERATINCKSSQSLLNARTGKNYLAWYQQKPGQPPKLLI | |
| | YWASTRESGVPDRFSGSGSGTDFTLTISSLQAEDVAVYYCKQSYSRRTFGCGT | |
| | KVEIKGGGGSGGGGSGGGGSGGGGSQVOLVOSGAEVKKPGASVKVSCKASGF | |
| | NIKDYYMHWVRQAPGQCLEWMGWIDLENANTIYDAKFQGRVTITRDTSASTA | |
| | YMELSSLRSEDTAVYYCARDAYGRYFYDVWGQGTLVTVSS (SEQ ID NO: 423) | |
| CNG-CD3- | QVQLVQSGAEVKKPGASVKVSCKASG | DIVMTQSPDSLAVSLGERATINCKS |
| 2 | FNIKDYYMHWVRQAPGQRLEWMGWI | SQSLLNARTGKNYLAWYQQKPGQP |
| | DLENANTIYDAKFQGRVTITRDTSAST | PKLLIYWASTRESGVPDRFSGSGSG |
| | AYMELSSLRSEDTAVYYCARDQYGRY | TDFTLTISSLQAEDVAVYYCKQSYS |
| | FYDVWGQGTLVTVSS (SEQ ID NO: | RRTFGGGTKVEIK (SEQ ID NO: 413) |
| | 424) | LCDR1: KSSQSLLNARTGKNYLA |
| | HCDR1*: FNIKDYYMH (SEQ ID NO: | (SEQ ID NO: 419) |
| | 414) | LCDR2: WASTRES (SEQ ID NO: 420) |
| | HCDR1: DYYMH (SEQ ID NO: 415) | LCDR3: KQSYSRRT (SEQ ID NO: |
| | HCDR2: WIDLENANTIYDAKFQG (SEQ | 421) |
| | ID NO: 416) | |
| | HCDR3*: ARDQYGRYFYDV (SEQ ID | |
| | NO: 425) | |
| | HCDR3: DQYGRYFYDV (SEQ ID NO: | |
| | 426) | |
| | scFv: | |
| | DIVMTQSPDSLAVSLGERATINCKSSQSLLNARTGKNYLAWYQQKPGQPPKLLI | |
| | YWASTRESGVPDRFSGSGSGTDFTLTISSLQAEDVAVYYCKQSYSRRTFGGGT | |
| | KVEIKGGGGSGGGGSGGGGSGGGGSQVOLVOSGAEVKKPGASVKVSCKASGF | |
| | NIKDYYMHWVRQAPGQRLEWMGWIDLENANTIYDAKFQGRVTITRDTSASTA | |
| | YMELSSLRSEDTAVYYCARDQYGRYFYDVWGQGTLVTVSS (SEQ ID NO: 427) | |
| | scFv with Cys substitutions: | |
| | DIVMTQSPDSLAVSLGERATINCKSSQSLLNARTGKNYLAWYQQKPGQPPKLLI | |
| | YWASTRESGVPDRFSGSGSGTDFTLTISSLQAEDVAVYYCKQSYSRRTFGCGT | |
| | KVEIKGGGGSGGGGSGGGGSGGGGSQVOLVOSGAEVKKPGASVKVSCKASGF | |
| | NIKDYYMHWVRQAPGQCLEWMGWIDLENANTIYDAKFQGRVTITRDTSASTA | |
| | YMELSSLRSEDTAVYYCARDQYGRYFYDVWGQGTLVTVSS (SEQ ID NO: 428) | |
| CNG-CD3- | QVQLVQSGAEVKKPGASVKVSCKASG | DIVMTQSPDSLAVSLGERATINCKS |
| 3 | FNIKDYYMHWVRQAPGQRLEWMGWI | SQSLLNARTGKNYLAWYQQKPGQP |
| | DLEEGNTIYDAKFQGRVTITRDTSAST | PKLLIYWASTRESGVPDRFSGSGSG |
| | AYMELSSLRSEDTAVYYCARDAYGRY | TDFTLTISSLQAEDVAVYYCKQSYS |
| | FYDVWGQGTLVTVSS (SEQ ID NO: | LRTFGGGTKVEIK (SEQ ID NO: 430) |
| | 429) | LCDR1: KSSQSLLNARTGKNYLA |
| | HCDR1*: FNIKDYYMH (SEQ ID NO: | (SEQ ID NO: 419) |
| | 414) | LCDR2: WASTRES (SEQ ID NO: 420) |
| | HCDR1: DYYMH (SEQ ID NO: 415) | LCDR3: KQSYSLRT (SEQ ID NO: |
| | HCDR2: WIDLEEGNTIYDAKFQG (SEQ | 432) |
| | ID NO: 431) | |
| | HCDR3*: ARDAYGRYFYDV (SEQ ID | |
| | NO: 417) | |
| | HCDR3: DAYGRYFYDV (SEQ ID NO: | |
| | 418) | |
| | scFv: | |
| | DIVMTQSPDSLAVSLGERATINCKSSQSLLNARTGKNYLAWYQQKPGQPPKLLI | |
| | YWASTRESGVPDRFSGSGSGTDFTLTISSLQAEDVAVYYCKQSYSLRTFGGGT | |
| | KVEIKGGGGSGGGGSGGGGSGGGGSQVQLVQSGAEVKKPGASVKVSCKASGF | |
| | NIKDYYMHWVRQAPGQRLEWMGWIDLEEGNTIYDAKFQGRVTITRDTSASTA | |
| | YMELSSLRSEDTAVYYCARDAYGRYFYDVWGQGTLVTVSS (SEQ ID NO: 433) | |
| | scFv with Cys substitutions: | |
| | DIVMTQSPDSLAVSLGERATINCKSSQSLLNARTGKNYLAWYQQKPGQPPKLLI | |
| | YWASTRESGVPDRFSGSGSGTDFTLTISSLQAEDVAVYYCKQSYSLRTFGCGT | |
| | KVEIKGGGGSGGGGSGGGGSGGGGSQVQLVQSGAEVKKPGASVKVSCKASGF | |
| | NIKDYYMHWVRQAPGQCLEWMGWIDLEEGNTIYDAKFQGRVTITRDTSASTA | |
| | YMELSSLRSEDTAVYYCARDAYGRYFYDVWGQGTLVTVSS (SEQ ID NO: 434) | |
| Adimab | QVQLVQSGAEVKKPGASVKVSCKASG | DIVMTQSPDSLAVSLGERATINCKS |
| mAb333 | FNIKDYYMHWVRQAPGQRLEWMGWI | SQSLLESRTGKNYLAWYQQKPGQP |
| | DLENANTIYDAKFQGRVTITRDTSAST | PKLLIYWASTRESGVPDRFSGSGSG |
| | AYMELSSLRSEDTAVYYCARDVYGRY | TDFTLTISSLQAEDVAVYYCKQSYS |
| | FYDLWGQGTLVTVSS (SEQ ID NO: | RRTFGGGTKVEIK (SEQ ID NO: 436) |
| | 435) | LCDR1: KSSQSLLNARTGKNYLA |
| | HCDR1: FNIKDYYMH (SEQ ID NO: 437) | (SEQ ID NO: 440) |
| | HCDR2: WIDLENANTIYDAKFQG (SEQ | LCDR2: WASTRES (SEQ ID NO: 441) |
| | ID NO: 438) | LCDR3: KQSYSRRT (SEQ ID NO: |
| | HCDR3: ARDVYGRYFYDL (SEQ ID NO: | 442) |
| | 439) | |
| Adimab | QVQLVQSGAEVKKPGASVKVSCKASG | DIVMTQSPDSLAVSLGERATINCKS |
| mAb334 | FNIKDYYMHWVRQAPGQRLEWMGWI | SQSLLNSRTGKNYLAWYQQKPGQP |
| | DLENANTIYDAKFQGRVTITRDTSAST | PKLLIYWASTRESGVPDRFSGSGSG |
| | AYMELSSLRSEDTAVYYCARDAYGGY | TDFTLTISSLQAEDVAVYYCKQSYS |
| | FYDVWGQGTLVTVSS (SEQ ID NO: | RRTFGCGTKVEIK (SEQ ID NO: 444) |
| | 443) | LCDR1: KSSQSLLNARTGKNYLA |
| | HCDR1: FNIKDYYMH (SEQ ID NO: 445) | (SEQ ID NO: 448) |
| | HCDR2: WIDLENANTIYDAKFQG (SEQ | LCDR2: WASTRES (SEQ ID NO: 449) |
| | ID NO: 446) | LCDR3: KQSYSRRT (SEQ ID NO: |
| | HCDR3: ARDAYGGYFYDV (SEQ ID | 450) |
| | NO: 447) | |
| Adimab | EVQLLESGGGLVQPGGSLRLSCAASGF | QTVVTQEPSLSVSPGGTVTLTCGSS |
| mAb404 | TFDTYAMNWVRQAPGKGLEWVARIR | TGAVTTSNYANWVQQTPGQAPRG |
| | SKYNNYATYYADSVKDRFTISRDDSKS | LIGGTDKRAPGVPDRFSGSLLGDKA |
| | TLYLQMESLRAEDTAVYYCVRHGNFG | ALTITGAQAEDEADYYCALWYSNH |
| | NYAVSWFAHWGQGTLVTVSS (SEQ ID | WVFGGGTKLTVL (SEQ ID NO: 452) |
| | NO: 451) | LCDR1: GSSTGAVTTSNYAN (SEQ |
| | HCDR1: FTFDTYAMN (SEQ ID NO: 453) | ID NO: 456) |
| | HCDR2: RIRSKYNNYATYYADSVKD | LCDR2: GTDKRAP (SEQ ID NO: 457) |
| | (SEQ ID NO: 454) | LCDR3: ALWYSNHWV (SEQ ID NO: |
| | HCDR3: VRHGNFGNYAVSWFAH (SEQ | 458) |
| | ID NO: 455) | |
| Adimab | EVQLLESGGGLVQPGGSLRLSCAASGF | QTVVTQEPSLSVSPGGTVTLTCGSS |
| mAb405 | TFDTYAMNWVRQAPGKGLEWVARIR | TGAVTTSNYANWVQQTPGQAPRG |
| | SKYNNYATYYADSVKDRFTISRDDSKS | LIGGTDKRAPGVPDRFSGSLLGDKA |
| | TLYLQMESLRAEDTAVYYCVRHGSFG | ALTITGAQAEDEADYYCALWYSNH |
| | NHIVSWFAHWGQGTLVTVSS (SEQ ID | WVFGGGTKLTVL (SEQ ID NO: 460) |
| | NO: 459) | LCDR1: GSSTGAVTTSNYAN (SEQ |
| | HCDR1: FTFDTYAMN (SEQ ID NO: 461) | ID NO: 464) |
| | HCDR2: RIRSKYNNYATYYADSVKD | LCDR2: GTDKRAP (SEQ ID NO: 465) |
| | (SEQ ID NO: 462) | LCDR3: ALWYSNHWV (SEQ ID NO: |
| | HCDR3: VRHGSFGNHIVSWFAH (SEQ | 466) |
| | ID NO: 463) | |
| Adimab | QVQLVQSGAEVKKPGASVKVSCKASG | DIVMTQSPDSLAVSLGERATINCKS |
| mAb-1 | FNIKDYYMHWVRQAPGQRLEWMGWI | SQSLLNSRTRKNYLAWYQQKPGQP |
| | DLENGNTIYDAKFQGRVTITRDTSAST | PKLLIYWASTRESGVPDRFSGSGSG |
| | AYMELSSLRSEDTAVYYCARDGYGRY | TDFTLTISSLQAEDVAVYYCKQSYS |
| | FYDVWGQGTLVTVSS (SEQ ID NO: | RRTFGGGTKVEIK (SEQ ID NO: 468) |
| | 467) | |
| Adimab | QVQLVQSGAEVKKPGASVKVSCKASG | DIVMTQSPDSLAVSLGERATINCKS |
| mAb-2 | FNIKDYYMHWVRQAPGQRLEWMGWI | SQSLLNNRTRKNYLAWYQQKPGQP |
| | DLENANTIYDAKFQGRVTITRDTSAST | PKLLIYWASTRESGVPDRFSGSGSG |
| | AYMELSSLRSEDTAVYYCARDVYGRY | TDFTLTISSLQAEDVAVYYCKQSYS |
| | LYDVWGQGTLVTVSS (SEQ ID NO: | RRTFGGGTKVEIK (SEQ ID NO: 470) |
| | 469) | |
| Adimab | QVQLVQSGAEVKKPGASVKVSCKASG | DIVMTQSPDSLAVSLGERATINCKS |
| mAb-3 | FNIKDYYMHWVRQAPGQRLEWMGWI | SQSLLNSRTRKNYLAWYQQKPGQP |
| | DLENGNTIYDPKFQGRVTITRDTSAST | PKLLIYWASTRESGVPDRFSGSGSG |
| | AYMELSSLRSEDTAVYYCARDAYGRY | TDFTLTISSLQAEDVAVYYCKQSYS |
| | FYDVWGQGTLVTVSS (SEQ ID NO: | RRTFGGGTKVEIK (SEQ ID NO: 472) |
| | 471) | |
| Adimab | QVQLVQSGAEVKKPGASVKVSCKASG | DIVMTQSPDSLAVSLGERATINCKS |
| mAb-4 | FNIKDYYMHWVRQAPGQRLEWMGWI | SQSLLNGRTRKNYLAWYQQKPGQP |
| | DLEEGNTIYDAKFQGRVTITRDTSAST | PKLLIYWASTRESGVPDRFSGSGTG |
| | AYMELSSLRSEDTAVYYCARDAYGRY | TDFTLTISSLQAEDVAVYYCKQSYS |
| | FYDVWGQGTLVTVSS (SEQ ID NO: | RRTFGGGTKVEIK (SEQ ID NO: 473) |
| | 429) | |
| Adimab | QVQLVQSGAEVKKPGASVKVSCKASG | DIVMTQSPDSLAVPLGERATINCKS |
| mAb-5 | FNIKDYYMHWVRQAPGQRLEWMGWI | SQSLLNSRTRKNYLAWYQQKPGQP |
| | DLENANTIYDAKFQGRVTITRDTSAST | PKLLIYWASTRESGVPDRFSGSGSG |
| | AYMELSSLRSEDTAVYYCARDNYGGY | TDFTLTISSLQAEDVAVYYCKQSYS |
| | FYDVWGQGTLVTVSS (SEQ ID NO: | RRTFGGGTKVEIK (SEQ ID NO: 475) |
| | 474) | |
| Adimab | QVQLVQSGAEVKKPGASVKVSCKASG | DIVMTQSPDSLAVSLGERATINCKS |
| mAb-6 | FNIKDYYMHWVRQAPGQRLEWMGWI | SQSLLNSRTRKNYLAWYQQKPGQP |
| | DLENGNTIYDPKFQGRVTITRDTSAST | PKLLIYWASTRESGVPDRFSGSGSG |
| | AYMELSSLRSEDTAVYYCARDGYGRY | TDFTLTISSLQAEDVAVYYCKQSYS |
| | FYDVWGQGTLVTVSS (SEQ ID NO: | LRTFGGGTKVEIK (SEQ ID NO: 477) |
| | 476) | |
| Adimab | QVQLVQSGAEVKKPGASVKVSCKASG | DIVMTQSPDSLAVSLGERATINCKS |
| mAb-7 | FNIKDYYMHWVRQAPGQRLEWMGWI | SQSLLNSRTRKNYLAWYQQKPGQP |
| | DLENGNTIYDPKFQGRVTITRDTSAST | PKLLIYWASTRESGVPDRFSGSGSG |
| | AYMELSSLRSEDTAVYYCARDGYGRY | TDFTLTISSLQAEDVAVYYCKQSYN |
| | FFDVWGQGTLVTVSS (SEQ ID NO: | LRTFGGGTKVEIK (SEQ ID NO: 479) |
| | 478) | |
| Adimab | QVQLVQSGAEVKKPGASVKVSCKASG | DIVMTQSPDSLAVSLGERATINCKS |
| mAb-8 | FNIKDYYMHWVRQAPGQRLEWMGWI | SQSLLNSRTRKNYLAWYQQKPGQP |
| | DLENGNTIYDPKFQGRVTITRDTSAST | PKLLIYWASTRESGVPDRFSGSGSG |
| | AYMELSSLRSEDTAVYYCAREGYGRY | TDFTLTISSLQAEDVAVYYCKQSYF |
| | FYDVWGQGTLVTVSS (SEQ ID NO: | RRAFGGGTKVEIK (SEQ ID NO: 481) |
| | 480) | |
| Adimab | QVQLVQSGAEVKKPGASVKVSCKASG | DIVMTQSPDSLAVSLGERATINCKS |
| mAb-9 | FNIKDYYMHWVRQAPGQRLEWMGWI | SQSLLNSRTRKNYLAWYQQKPGQP |
| | DLENGNTIYDPKFQGRVTITRDTSAST | PKLLIYWASTRESGVPDRFSGSGSG |
| | AYMELSSLRSEDTAVYYCARDGYGRY | TDFTLTISSLQAEDVAVYYCKQSYN |
| | YYDVWGQGTLVTVSS (SEQ ID NO: | LRTFGGGTKLEIK (SEQ ID NO: 483) |
| | 482) | |
| Adimab | QVQLVQSGAEVKKPGASVKVSCKASG | DIVMTQSPDSLAVSLGERATINCKS |
| mAb-10 | FNIKDYYMHWVRQAPGQRLEWMGWI | SQSLLNSRTRKNYLAWYQQKPGQS |
| | DLENGNTIYQPKFQGRVTITRDTSAST | PKLLIYWASTRESGVPDRFTGSGSG |
| | AYMELSSLRSEDTAVYYCARDGYGRY | TDFTLTISSLQAEDVAVYYCKQSYS |
| | FYDVWGQGTLVTVSS (SEQ ID NO: | LRTFGGGTKVEIK (SEQ ID NO: 485) |
| | 484) | |
| Adimab | QVQLVQSGAEVKKPGASVKVSCKASG | DIVMTQSPDSLAVSLGERATINCKS |
| mAb-11 | FNIKDYYMHWVRQAPGQRLEWMGWI | SQSLLESRTGKNYLAWYQQKPGQP |
| | DLENGNTIYDPKFQGRVTITRDTSAST | PKLLIYWASTRESGVPDRFSGSGSG |
| | AYMELSSLRSEDTAVYYCARDGYGRY | TDFTLTISSLQAEDVAVYYCKQSYS |
| | FYDYWGQGTLVTVSS (SEQ ID NO: | LRTFGGGTKVEIK (SEQ ID NO: 487) |
| | 486) | |
| CD3 | DIKLQQSGAELARPGASVKMSCKTSG | VDDIQLTQSPAIMSASPGEKVTMTC |
| binding | YTFTRYTMHWVKQRPGQGLEWIGYIN | RASSSVSYMNWYQQKSGTSPKRWI |
| domain in | PSRGYTNYNQKFKDKATLTTDKSSSTA | YDTSKVASGVPYRFSGSGSGTSYSL |
| blinatumo | YMQLSSLTSEDSAVYYCARYYDDHYC | TISSMEAEDAATYYCQQWSSNPLTF |
| mab | LDYWGQGTTLTVSSVE (SEQ ID NO: | GAGTKLELK (SEQ ID NO: 489) |
| | 488) | |
| | scFv: | |
| | DIKLQQSGAELARPGASVKMSCKTSGYTFTRYTMHWVKQRPGQGLEWIGYIN | |
| | PSRGYTNYNQKFKDKATLTTDKSSSTAYMQLSSLTSEDSAVYYCARYYDDHY | |
| | CLDYWGQGTTLTVSSVEGGSGGSGGSGGSGGVDDIQLTQSPAIMSASPGEKVT | |
| | MTCRASSSVSYMNWYQQKSGTSPKRWIYDTSKVASGVPYRFSGSGSGTSYSLT | |
| | ISSMEAEDAATYYCQQWSSNPLTFGAGTKLELK (SEQ ID NO: 490) | |
| Novimmune | QVQLVESGGGVVQPGRSLRLSCAASG | EIVLTQSPATLSLSPGERATLSCRAS |
| 28F11 | FKFSGYGMHWVRQAPGKGLEWVAVI | QSVSSYLAWYQQKPGQAPRLLIYD |
| | WYDGSKKYYVDSVKGRFTISRDNSKN | ASNRATGIPARFSGSGSGTDFTLTIS |
| | TLYLQMNSLRAEDTAVYYCARQMGY | SLEPEDFAVYYCQQRSNWPPLTFG |
| | WHFDLWGRGTLVTVSS (SEQ ID NO: | GGTKVEIK (SEQ ID NO: 492) |
| | 491) | LCDR1: RASQSVSSYLA (SEQ ID |
| | HCDR1: GYGMH (SEQ ID NO: 493) | NO: 496) |
| | HCDR2: VIWYDGSKKYYVDSVKG | LCDR2: DASNRAT (SEQ ID NO: 497) |
| | (SEQ ID NO: 494) | LCDR3: QQRSNWPPLT (SEQ ID NO: |
| | HCDR3: QMGYWHFDL (SEQ ID NO: | 498) |
| | 495) | |
| Novimmune | EVQLLESGGGLVQPGGSLRLSCAASGF | DFMLTQPHSVSESPGKTVIISCWYQ |
| 27H5 | TFSSFPMAWVRQAPGKGLEWVSTISTS | QRPGRAPTTVIFGVPDRFSGSIDRSS |
| | GGRTYYRDSVKGRFTISRDNSKNTLYL | NSASLTISGLQTEDEADYYCFGGGT |
| | QMNSLRAEDTAVYYCAKFRQYSGGFD | KLTVLGQPKAAPSVTLFPPSSEELQ |
| | YWGQGTLVTVSS (SEQ ID NO: 499) | (SEQ ID NO: 500) |
| Glaxo mAb | EVQLLESGGGLVQPGGSLRLSCAASGF | DIQLTQPNSVSTSLGSTVKLSCTLSS |
| | TFSSFPMAWVRQAPGKGLEWVSTISTS | GNIENNYVHWYQLYEGRSPTTMIY |
| | GGRTYYRDSVKGRFTISRDNSKNTLYL | DDDKRPDGVPDRFSGSIDRSSNSAF |
| | QMNSLRAEDTAVYYCAKFRQYSGGFD | LTIHNVAIEDEAIYFCHSYVSSFNVF |
| | YWGQGTLVTVSS (SEQ ID NO: 501) | GGGTKLTVLR (SEQ ID NO: 502) |
| Eureka | DVQLVQSGAEVKKPGASVKVSCKASG | DIVLTQSPATLSLSPGERATLSCRAS |
| mAb | YTFTRYTMHWVRQAPGQGLEWIGYIN | QSVSYMNWYQQKPGKAPKRWIYD |
| | PSRGYTNYADSVKGRFTITTDKSTSTA | TSKVASGVPARFSGSGSGTDYSLTI |
| | YMELSSLRSEDTATYYCARYYDDHYC | NSLEAEDAATYYCQQWSSNPLTFG |
| | LDYWGQGTPVTVSS (SEQ ID NO: 503) | GGTKVEIK (SEQ ID NO: 504) |
| | scFv: | |
| | DVQLVQSGAEVKKPGASVKVSCKASGYTFTRYTMHWVRQAPGQGLEWIGYI | |
| | NPSRGYTNYADSVKGRFTITTDKSTSTAYMELSSLRSEDTATYYCARYYDDHY | |
| | CLDYWGQGTTVTVSSGEGTSTGSGGSGGSGGADDIVLTQSPATLSLSPGERAT | |
| | LSCRASQSVSYMNWYQQKPGKAPKRWIYDTSKVASGVPARFSGSGSGTDYSL | |
| | TINSLEAEDAATYYCQQWSSNPLTFGGGTKVEIK (SEQ ID NO: 505) | |
| Muromonab | QVQLVQSGGGVVQPGRSLRLSCKASG | DDIQMTQSPSSLSASVGDRVTITCS |
| | YTFTRYTMHWVRQAPGKGLEWIGYIN | ASSSVSYMNWYQQTPGKAPKRWIY |
| | PSRGYTNYNQKVKDRFTISRDNSKNTA | DTSKLASGVPSRFSGSGSGTDYTFTI |
| | FLQMDSLRPEDTGVYFCARYYDDHYC | SSLQPEDIATYYCQQWSSNPFTFGQ |
| | LDYWGQGTPVTVSS (SEQ ID NO: 506) | GTKLQIT (SEQ ID NO: 507) |
| MacroGenics | QVQLVQSGGGVVQPGRSLRLSCKASG | DIQMTQSPSSLSASVGDRVTITCSAS |
| mAb | YTFTRYTMHWVRQAPGKGLEWIGYIN | SSVSYMNWYQQTPGKAPKRWIYD |
| humanized | PSRGYTNYNQKFKDRFTISTDKSKSTA | TSKLASGVPSRFSGSGSGTDYTFTIS |
| OKT3 | FLQMDSLRPEDTAVYYCARYYDDHYC | SLQPEDIATYYCQQWS SNPFTFGQG |
| | LDYWGQGTPVTVSS (SEQ ID NO: 508) | TKLQITR (SEQ ID NO: 509) |
| Roche | EVQLLESGGGLVQPGGSLRLSCAASGF | QAVVTQEPSLTVSPGGTVTLTCGSS |
| CH2527 | TFSTYAMNWVRQAPGKGLEWVSRIRS | TGAVTTSNYANWVQEKPGQAFRG |
| | KYNNYATYYADSVKGRFTISRDDSKN | LIGGTNKRAPGTPARFSGSLLGGKA |
| | TLYLQMNSLRAEDTAVYYCVRHGNFG | ALTLSGAQPEDEAEYYCALWYSNL |
| | NSYVSWFAYWGQGTLVTVSS (SEQ ID | WVFGGGTKLTVL (SEQ ID NO: 601) |
| | NO: 600) | |
| Regeneron | EVQLVESGGGLVQPGRSLRLSCAASGF | AEIVMTQSPATLSVSPGERATLSCR |
| mAb (anti- | TFDDYTMHWVRQAPGKGLEWVSGIS | ASQSVSSNLAWYQQKPGQAPRLLI |
| CD3/anti- | WNSGSIGYADSVKGRFTISRDNAKKSL | YGASTRATGIPARFSGSGSGTEFTLT |
| CD20) | YLQMNSLRAEDTALYYCAKDNSGYG | ISSLQSEDFAVYYCQHYINWPLTFG |
| | HYYYGMDVWGQGTTVTVAS (SEQ ID | GGTKVEIK (SEQ ID NO: 603) |
| | NO: 602) | |
| WuXi | QVQLVQSGAEVKKPGSSVKVSCKASG | DIVMTQSPDSLAVSLGERATINCKS |
| WBP3311_ | YSFTTYYIHWVRQAPGQGLEWMGWIF | SQSLLNSRTRKNYLAWYQQKPGQP |
| 2.166.48-z1 | PGNDNIKYSEKFKGRVTITADKSTSTA | PKLLIYWASTRKSGVPDRFSGSGSG |
| | YMELSSLRSEDTAVYYCAIDSVSIYYF | TDFTLTISSLQAEDVAVYYCTQSFIL |
| | DYWGQGTLVTVSS (SEQ ID NO: 604) | RTFGGGTKVEIK (SEQ ID NO: 605) |
| | HCDR1: GYSFTTYYIH (SEQ ID NO: | LCDR1: KSSQSLLNSRTRKNYLA |
| | 606) | (SEQ ID NO: 609) |
| | HCDR2: WIFPGNDNIKYSEKFKG (SEQ | LCDR2: WASTRKS (SEQ ID NO: 610) |
| | ID NO: 607) | LCDR3: TQSFILRT (SEQ ID NO: 611) |
| | HCDR3: DSVSIYYFDY (SEQ ID NO: | |
| | 608) | |
| WuXi | QVQLVQSGAEVKKPGSSVKVSCKASG | DIVMTQSPDSLAVSLGERATINCKS |
| WBP3311_ | FAFTDYYIHWVRQAPGQGLEWMGWIS | SQSLLNSRTRKNYLAWYQQKPGQP |
| 2.306.4-z1 | PGNVNTKYNENFKGRVTITADKSTSTA | PKLLIYWASTRQSGVPDRFSGSGSG |
| | YMELSSLRSEDTAVYYCARDGYSLYY | TDFTLTISSLQAEDVAVYYCTQSHT |
| | FDYWGQGTLVTVSS (SEQ ID NO: 612) | LRTFGGGTKVEIK (SEQ ID NO: 613) |
| | HCDR1: GFAFTDYYIH (SEQ ID NO: | LCDR1: KSSQSLLNSRTRKNYLA |
| | 614) | (SEQ ID NO: 617) |
| | HCDR2: WISPGNVNTKYNENFKG (SEQ | LCDR2: WASTRQS (SEQ ID NO: 618) |
| | ID NO: 615) | LCDR3: TQSHTLRT (SEQ ID NO: |
| | HCDR3: DGYSLYYFDY (SEQ ID NO: | 619) |
| | 616) | |
| ADLQ | MAESGGGSVQTGGSLRLSCAYTASSV | N/A |
| mAb-1 | CMAWFRQAPGKEREGVAVTREGLTKT | |
| | GYADSVKGRFAISQDYAKKTLYLQMS | |
| | SLKPEDTARYYCAARPTSPCTVDGELL | |
| | ASTYNYWGQGTQVTV (SEQ ID NO: | |
| | 620) | |
| ADLQ | MAESGGGSVQTGGSLRLSCAYTASSV | N/A |
| mAb-2 | CMAWFRQAPGKEREGVAVTREGLTKT | |
| | GYADSVKGRFAISQDYAKKTLYLQMS | |
| | SLKPEDTARYYCAARPTSPCTVDGELL | |
| | ASTYDYWGQGTQVTV (SEQ ID NO: | |
| | 621) | |
| ADLQ | MAESGGGSVQTGGSLRLSCAYTASSV | N/A |
| mAb-3 | CMAWFRQAPGKEREGVAVTREGLTQT | |
| | GYADSVKGRFAISQDYAKKTLYLQMS | |
| | SLKPEDTARYYCAARPTSPCTVDGELL | |
| | ASTYNYWGQGTQVTV (SEQ ID NO: | |
| | 622) | |
| ADLQ | MAESGGGSVQTGGSLRLSCAYTASSV | N/A |
| mAb-4 | CMAWFRQAPGKEREGVAVTREGLTQT | |
| | GYADSVKGRFAISQDYAKKTLYLQMS | |
| | SLKPEDTARYYCAARPTSPCTVDGELL | |
| | ASTYDYWGQGTQVTV (SEQ ID NO: | |
| | 623) | |

Where the VL and LCDR sequences are noted as “N/A,” the antigen-binding site is an sdAb having a VH (e.g., VHH) only.

In certain embodiments, the second antigen-binding site comprises a VH that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VH of an antibody disclosed in Table 3, and a VL that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the VL of the same antibody disclosed in Table 3. In certain embodiments, the antigen-binding site comprises the HCDR1, HCDR2, HCDR3, LCDR1, LCDR2, and LCDR3, determined under Kabat (see Kabat et al., (1991) Sequences of Proteins of Immunological Interest, NIH Publication No. 91-3242, Bethesda), Chothia (see, e.g., Chothia C & Lesk A M, (1987), J Mol Biol 196: 901-917), MacCallum (see MacCallum R M et al., (1996) J Mol Biol 262: 732-745), IMGT (see Lefranc, (1999) The Immunologist, 7, 132-136), or any other CDR determination method known in the art, of the VH and/or VL sequences of an antibody disclosed in Table 3. In certain embodiments, the antigen-binding site comprises the HCDR1, HCDR2, HCDR3, LCDR1, LCDR2, and LCDR3 sequences of an antibody disclosed in Table 3. In certain embodiments, the antigen-binding site comprises the VH and VL sequences of an antibody disclosed in Table 3.

In certain embodiments, the second antigen-binding site that binds CD3 is derived from CNG-CD3-1. In certain embodiments, the second antigen-binding site comprises a VH comprising HCDR1, HCDR2, and HCDR3 sequences set forth in SEQ ID NOs: 414, 416, and 417, respectively, and a VL comprising LCDR1, LCDR2, and LCDR3 sequences set forth in SEQ ID NOs: 419, 420, and 421, respectively. In certain embodiments, the second antigen-binding site comprises a VH comprising HCDR1, HCDR2, and HCDR3 sequences set forth in SEQ ID NOs: 415, 416, and 418, respectively, and a VL comprising LCDR1, LCDR2, and LCDR3 sequences set forth in SEQ ID NOs: 419, 420, and 421, respectively. In certain embodiments, the second antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 412, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 413. In certain embodiments, the VH and the VL of the second antigen-binding site comprise the amino acid sequences of SEQ ID NOs: 412 and 413, respectively.

In certain embodiments, the second antigen-binding site that binds CD3 is derived from CNG-CD3-2. In certain embodiments, the second antigen-binding site comprises a VH comprising HCDR1, HCDR2, and HCDR3 sequences set forth in SEQ ID NOs: 414, 416, and 425, respectively, and a VL comprising LCDR1, LCDR2, and LCDR3 sequences set forth in SEQ ID NOs: 419, 420, and 421, respectively. In certain embodiments, the second antigen-binding site comprises a VH comprising HCDR1, HCDR2, and HCDR3 sequences set forth in SEQ ID NOs: 415, 416, and 426, respectively, and a VL comprising LCDR1, LCDR2, and LCDR3 sequences set forth in SEQ ID NOs: 419, 420, and 421, respectively. In certain embodiments, the second antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 424, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 413. In certain embodiments, the VH and the VL of the second antigen-binding site comprise the amino acid sequences of SEQ ID NOs: 424 and 413, respectively.

In certain embodiments, the second antigen-binding site that binds CD3 is derived from CNG-CD3-3. In certain embodiments, the second antigen-binding site comprises a VH comprising HCDR1, HCDR2, and HCDR3 sequences set forth in SEQ ID NOs: 414, 431, and 417, respectively, and a VL comprising LCDR1, LCDR2, and LCDR3 sequences set forth in SEQ ID NOs: 419, 420, and 432, respectively. In certain embodiments, the second antigen-binding site comprises a VH comprising HCDR1, HCDR2, and HCDR3 sequences set forth in SEQ ID NOs: 415, 431, and 418, respectively, and a VL comprising LCDR1, LCDR2, and LCDR3 sequences set forth in SEQ ID NOs: 419, 420, and 432, respectively. In certain embodiments, the second antigen-binding site comprises a VH comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 429, and a VL that comprising an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to SEQ ID NO: 430. In certain embodiments, the VH and the VL of the second antigen-binding site comprise the amino acid sequences of SEQ ID NOs: 429 and 430, respectively.

Such antigen-binding site may take the form of scFv. In certain embodiments, the VH is positioned C-terminal to the VL. In certain embodiments, the VH is positioned N-terminal to the VL. In certain embodiments, the VH and the VL are linked by a peptide linker, for example, a linker disclosed in subsection D below titled “Linkers.” In certain embodiments, the second antigen-binding site comprises the amino acid sequence of SEQ ID NO: 422, 427, or 433. To stabilize the scFv, the amino acid residues at position 44 of the VH and at position 100 of the VL (under Kabat numbering) can be substituted by Cys, thereby facilitating the formation of a disulfide bond between the VH and the VL. Accordingly, in certain embodiments, the VH and VL comprise Cys at positions 100 and 44, respectively. In certain embodiments, the second antigen-binding site comprises the amino acid sequence of SEQ ID NO: 423, 428, or 434.

In other embodiments, the second antigen-binding site comprises an sdAb comprising a VH comprising complementarity determining regions HCDR1, HCDR2, and HCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an sdAb antibody provided in Table 3. In certain embodiments, the VH comprises the HCDR1, HCDR2, and HCDR3 sequences of the antibody provided in Table 3. In certain embodiments, the VH comprises the amino acid sequence of the VH of an sdAb provided in Table 3.

In certain embodiments, the second antigen-binding site competes for binding CD3 (e.g., human CD3 and/or Macaca CD3) with an antibody or antigen-binding fragment thereof comprising the VH, VL and/or scFv sequences provided in Table 3.

In certain embodiments, the second antigen-binding site of the multi-specific binding protein binds CD3 (e.g., human CD3 and/or Macaca CD3) with a dissociation constant (K_{D}) of about 0.1 nM-about 1 μM. The K_{D} can be measured by a method known in the art. In certain embodiments, the K_{D} is measured by SPR to CD3 or an extracellular fragment thereof immobilized on a chip. In certain embodiments, the K_{D} is measured by flow cytometry to CD3 expressed on the surface of cells, for example, following the method described in Example 6 below.

In certain embodiments, the second antigen-binding site binds CD3 with a K_{D} lower than or equal to 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, or 10 pM. For example, in certain embodiments, the first antigen-binding site binds CD3 with a K_{D} about 10 pM-about 1 nM, about 10 pM-about 0.9 nM, about 10 pM-about 0.8 nM, about 10 pM-about 0.7 nM, about 10 pM-about 0.6 nM, about 10 pM nM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, or about 9 nM-about 10 nM.

It is understood that a greater K_{D} (i.e., a lower affinity to CD3) may be desirable in the context of a multi-specific binding protein. Without wishing to be bound by theory, it is contemplated that a multi-specific binding protein having a very high affinity to CD3 may result in excessive cytokine release which narrows the therapeutic window. Accordingly, in certain embodiments, the second antigen-binding site binds CD3 (e.g., human CD3, e.g., human CD3ε) with a K_{D} greater than or equal to 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, or 100 nM. In certain embodiments, the second antigen-binding site binds CD3 with a K_{D} about 1 nM-about 100 nM, about 1 nM-about 90 nM, about 1 nM-about 80 nM, about 1 nM-about 70 nM, about 1 nM-about 60 nM, about 1 nM-about 50 nM, about 1 nM-about 40 nM, about 1 nM-about 30 nM, about 1 nM-about 20 nM, about 1 nM-about 10 nM, about 10 nM-about 100 nM, about 10 nM-about 90 nM, about 10 nM-about 80 nM, about 10 nM-about 70 nM, about 10 nM-about 60 nM, about 10 nM-about 50 nM, about 10 nM-about 40 nM, about 10 nM-about 30 nM, or about 10 nM-about 20 nM.

It is understood that the binding affinity to CD3 of the second antigen-binding site alone may be different from the binding affinity of the same antigen-binding site in the context of the multi-specific binding protein disclosed herein, possibly due to the conformational restraint from the other domains. The context-dependent binding affinity is described in the subsection E below titled “Binding Affinity”

In certain embodiments, the second antigen-binding site, when present in the form of an Fab, has a melting temperature of at least 60° C., at least 65° C., at least 70° C., at least 75° C., or at least 80° C. In certain embodiments, the second antigen-binding site, when present in the form of an Fab, has a melting temperature in the range of 60-85° C., 60-80° C., 60-75° C., 60-70° C., 60-65° C., 65-85° C., 65-80° C., 65-75° C., 65-70° C., 70-85° C., 70-80° C., 70-75° C., 75-85° C., 75-80° C., or 80-85° C.

### C. Construct Formats

The first, second, and third antigen-binding sites may take various forms. In certain embodiments, the first, second, and/or third antigen-binding sites comprises two antibody variable domains (e.g., a VH and a VL). The VH and the VL can be mutated to introduce a disulfide bond (e.g., between H44 and L100) that stabilizes the antigen-binding site (see, Zhao et al. (2010) Int. J. Mol. Sci., 12(1):1-11). In certain embodiments, the first, second, and/or third antigen-binding sites comprises a single antibody variable domain (e.g., an sdAb).

In an antigen-binding site that contains a VH and a VL, the VH and the VL can be linked to form an scFv. The VH can be positioned N-terminal or C-terminal to the VL. The VH and the VL are typically linked through a linker, such as a peptide linker. Exemplary sequences of peptide linkers are provided in subsection D below titled “Linkers.” In certain embodiments, the VH of an antigen-binding domain is connected to the VL of the antigen-binding domain through a peptide linker having an amino acid sequence listed in Table 4. In particular embodiments, the VH of an antigen-binding domain is connected to the VL of the antigen-binding domain through a peptide linker having the amino acid sequence of SEQ ID NO: 298, 299, or 302, wherein the VH is positioned N-terminal to the VL. In other particular embodiments, the VH of an antigen-binding domain is connected to the VL of the antigen-binding domain through a peptide linker having the amino acid sequence of SEQ ID NO: 298, 299, or 302, wherein the VH is positioned C-terminal to the VL.

Alternatively, the VH and the VL may be present on separate polypeptide chains, and the formation of a VH-VL complex may be facilitated by additional domains, such as antibody constant regions CH1 and CL. Accordingly, in certain embodiments, the multi-specific binding protein comprises an Fab comprising a VH and a VL disclosed herein.

In certain embodiments, a multi-specific binding protein of the present invention comprises a first antigen-binding site comprising a single antibody variable domain, a second antigen-binding site comprising a single antibody variable domain, and a third antigen-binding site comprising a single antibody variable domain. In certain embodiments, the multi-specific binding protein comprises a first antigen-binding site in an sdAb format, a second antigen-binding site in an sdAb format, and a third antigen-binding site in an sdAb format.

In certain embodiments, a multi-specific binding protein of the present invention comprises a first antigen-binding site comprising a single antibody variable domain, a second antigen-binding site comprising a single antibody variable domain, and a third antigen-binding site comprising two antibody variable domains. In certain embodiments, the multi-specific binding protein comprises a first antigen-binding site in an sdAb format, a second antigen-binding site in an sdAb format, and a third antigen-binding site in an scFv format.

In certain embodiments, a multi-specific binding protein of the present invention comprises a first antigen-binding site comprising a single antibody variable domain, a second antigen-binding site comprising two antibody variable domains, and a third antigen-binding site comprising a single antibody variable domain. In certain embodiments, the multi-specific binding protein comprises a first antigen-binding site in an sdAb format, a second antigen-binding site in an scFv format, and a third antigen-binding site in an sdAb format.

In certain embodiments, a multi-specific binding protein of the present invention comprises a first antigen-binding site comprising a single antibody variable domain, a second antigen-binding site comprising two antibody variable domains, and a third antigen-binding site comprising two antibody variable domains. In certain embodiments, the multi-specific binding protein comprises a first antigen-binding site in an sdAb format, a second antigen-binding site in an scFv format, and a third antigen-binding site in an scFv format.

In certain embodiments, a multi-specific binding protein of the present invention comprises a first antigen-binding site comprising two antibody variable domains, a second antigen-binding site comprising a single antibody variable domain, and a third antigen-binding site comprising a single antibody variable domain. In certain embodiments, the multi-specific binding protein comprises a first antigen-binding site in an scFv format, a second antigen-binding site in an sdAb format, and a third antigen-binding site in an sdAb format.

In certain embodiments, a multi-specific binding protein of the present invention comprises a first antigen-binding site comprising two antibody variable domains, a second antigen-binding site comprising a single antibody variable domain, and a third antigen-binding site comprising two antibody variable domains. In certain embodiments, the multi-specific binding protein comprises a first antigen-binding site in an scFv format, a second antigen-binding site in an sdAb format, and a third antigen-binding site in an scFv format.

In certain embodiments, a multi-specific binding protein of the present invention comprises a first antigen-binding site comprising two antibody variable domains, a second antigen-binding site comprising two antibody variable domains, and a third antigen-binding site comprising a single antibody variable domain. In certain embodiments, the multi-specific binding protein comprises a first antigen-binding site in an scFv format, a second antigen-binding site in an scFv format, and a third antigen-binding site in an sdAb format.

In certain embodiments, a multi-specific binding protein of the present invention comprises a first antigen-binding site comprising two antibody variable domains, a second antigen-binding site comprising two antibody variable domains, and a third antigen-binding site comprising two antibody variable domains. In certain embodiments, the multi-specific binding protein comprises a first antigen-binding site in an scFv format, a second antigen-binding site in an scFv format, and a third antigen-binding site in an scFv format.

The three antigen-binding sites of the multi-specific binding protein can be linked in any one of the following orientations in an amino-to-carboxyl direction:

(i) the first antigen-binding site (e.g., CD19 binding domain)—the second antigen-binding site (e.g., CD3 binding domain)—the third antigen-binding site (serum albumin binding domain);
(ii) the first antigen-binding site (e.g., CD19 binding domain)—the third antigen-binding site (serum albumin binding domain)—the second antigen-binding site (e.g., CD3 binding domain);
(iii) the second antigen-binding site (e.g., CD3 binding domain)—the first antigen-binding site (e.g., CD19 binding domain)—the third antigen-binding site (serum albumin binding domain);
(iv) the second antigen-binding site (e.g., CD3 binding domain)—the third antigen-binding site (serum albumin binding domain)—the first antigen-binding site (e.g., CD19 binding domain);
(v) the third antigen-binding site (serum albumin binding domain)—the first antigen-binding site (e.g., CD19 binding domain)—the second antigen-binding site (e.g., CD3 binding domain); and
(vi) the third antigen-binding site (serum albumin binding domain)—the second antigen-binding site (e.g., CD3 binding domain)—the first antigen-binding site (e.g., CD19 binding domain), wherein the dashes above represent a peptide bond and/or a linker (e.g., peptide linker).

In certain embodiments, the third antigen-binding site is not positioned between the first antigen-binding site and the second antigen-binding site. It is contemplated that constructs having such formats have favorable therapeutic efficacy and in vivo half-life. In certain embodiments, the third antigen-binding site is positioned N-terminal to both the first antigen-binding site and the second antigen-binding site or C-terminal to both the first antigen-binding site and the second antigen-binding site. In certain embodiments, the third antigen-binding site is positioned N-terminal to both the first antigen-binding site and the second antigen-binding site. In certain embodiments, the third antigen-binding site is positioned C-terminal to both the first antigen-binding site and the second antigen-binding site.

The position (N-terminal or C-terminal) of one antigen-binding site relative to another is determined under the definitions of “N-terminal” and “C-terminal” as known in the art if a single polypeptide chain comprises both antigen-binding sites. It is understood that if an antigen-binding site comprises two separate polypeptide chains, its position (N-terminal or C-terminal) relative to another antigen-binding site (either having a single polypeptide chain or two polypeptide chains) can be similarly determined if a single polypeptide chain comprises at least one polypeptide chain of the former and at least one polypeptide chain of the latter. It is further understood that if antigen-binding site A is N-terminal to antigen-binding site B and antigen-binding site B is N-terminal to antigen-binding site C, it is deemed that antigen-binding site A is positioned N-terminal to antigen-binding site C even if antigen-binding sites A and C are not present in any single, common polypeptide chain. More complex structures of multi-specific binding proteins are also contemplated, some of which may have orientations difficult to characterize using the terms of “N-terminal” and “C-terminal” as described above due to, for example, different relative positions of two antigen-binding sites on one polypeptide chain versus another polypeptide chain, or the presence of a loop structure.

According to the present invention, the multi-specific binding proteins and their constituent binding domains are in the form of one or more polypeptides. Such polypeptides may include proteinaceous parts and non-proteinaceous parts (e.g., chemical linkers or chemical cross-linking agents such as glutaraldehyde). In certain embodiments, a multi-specific binding protein of the present invention includes a first antigen-binding site, a second antigen-binding site, and a third antigen-binding site, all of which are linked together to form a single polypeptide chain. In certain embodiments, the first, second, and third antigen-binding sites take the forms of scFv and/or sdAb, for example, in a combination as described above, to form a single polypeptide chain.

### D. Linkers

As noted above, the antigen-binding sites of the multi-specific binding proteins of the present invention can be linked through a peptide bond or a linker (e.g., peptide linker). In certain embodiments, at least two adjacent antigen-binding sites are connected by a linker (e.g., peptide linker). In certain embodiments, each two adjacent antigen-binding sites are connected by a linker (e.g., peptide linker).

In certain embodiments, the three antigen-binding sites of the multi-specific binding protein can be linked by linkers (e.g., peptide linkers) denoted as L₁ and L₂ in any one of the following orientations in an amino-to-carboxyl direction:

(i) the first antigen-binding site (e.g., CD19 binding domain)—L₁— the second antigen-binding site (e.g., CD3 binding domain)—L₂— the third antigen-binding site (serum albumin binding domain);
(ii) the first antigen-binding site (e.g., CD19 binding domain)—L₁— the third antigen-binding site (serum albumin binding domain)—L₂— the second antigen-binding site (e.g., CD3 binding domain);
(iii) the second antigen-binding site (e.g., CD3 binding domain)—L₁— the first antigen-binding site (e.g., CD19 binding domain)—L₂— the third antigen-binding site (serum albumin binding domain);
(iv) the second antigen-binding site (e.g., CD3 binding domain)—L₁— the third antigen-binding site (serum albumin binding domain)—L₂— the first antigen-binding site (e.g., CD19 binding domain);
(v) the third antigen-binding site (serum albumin binding domain)—L₁— the first antigen-binding site (e.g., CD19 binding domain)—L₂— the second antigen-binding site (e.g., CD3 binding domain); and
(vi) the third antigen-binding site (serum albumin binding domain)—L₁— the second antigen-binding site (e.g., CD3 binding domain)—L₂— the first antigen-binding site (e.g., CD19 binding domain).

It is appreciated that in a given construct, L₁, L₂, or both L₁ and L₂ may be replaced with a peptide bond.

It is understood that if a single polypeptide chain comprises two adjacent antigen-binding sites, the peptide linker connecting the two antigen-binding sites represents the amino acid sequence between them. If an antigen-binding site comprises two separate polypeptide chains, one of which is present in a single, common polypeptide as an adjacent antigen-binding site or a polypeptide chain thereof, the peptide linker connecting the two antigen-binding sites represents the amino acid sequence between them in the common, single polypeptide.

In certain embodiments, the linkers L₁ and L₂ are peptide linkers. Suitable lengths of L₁ and L₂ can be independently selected. For example, in certain embodiments, L₁ and/or L₂ are about 50 or less amino acid residues in length. In certain embodiments, L₁ consists of about 50 or less amino acid residues. In certain embodiments, L₁ consists of about 20 or less amino acid residues. In certain embodiments, L₂ consists of about 50 or less amino acid residues. In certain embodiments, L₂ consists of about 20 or less amino acid residues. In certain embodiments, L₁ and L₂ independently consist of about 50 or less amino acid residues. In certain embodiments, L₁ and L₂ independently consist of about 20 or less amino acid residues.

In some embodiments, peptide linkers L₁ and L₂ have an optimized length and/or amino acid composition. In some embodiments, L₁ and L₂ are of the same length and have the same amino acid composition. In other embodiments, L₁ and L₂ are different. In certain embodiments, L₁ and/or L₂ are “short,” i.e., consist of 1, 2, 3, 4, 5, 6, 7, 8, 9, 10, 11 or 12 amino acid residues. Thus, in certain instances, the linkers consist of about 12 or less amino acid residues. In certain embodiments, L₁ and/or L₂ are “long,” e.g., consist of 15, 20 or 25 amino acid residues. In some embodiments, L₁ and/or L₂ consist of about 3 to about 15, for example 8, 9 or 10 contiguous amino acid residues.

Regarding the amino acid composition of L₁ and L₂, peptides are selected with properties that confer flexibility to multi-specific binding protein of the present invention, do not interfere with the binding domains as well as resist cleavage from proteases. For example, glycine and serine residues generally provide protease resistance. Examples of the linkers suitable for linking the domains in the multi-specific binding protein include but are not limited to (GS)ₙ, (GGS)ₙ, (GGGS)ₙ, (GGSG)ₙ, (GGSGG)ₙ, and (GGGGS)ₙ, wherein n is 1, 2, 3, 4, 5, 6, 7, 8, 9, 10, 11, 12, 13, 14, 15, 16, 17, 18, 19, or 20. In some embodiments, L₁ and/or L₂ are independently selected from the peptide sequences listed in Table 4. In some embodiments, L₁ and/or L₂ are independently selected from SEQ ID NOs: 292, 293, 294, 295, 296, 297, 298, 299, 300, 301, or 302. In some embodiments, L₁ and/or L₂ are independently selected from SEQ ID NOs: 298, 299, and 302. In some embodiments, L₁ and/or L₂ comprise the amino acid sequence of SEQ ID NO: 298, 299, or 302. In some embodiments, L₁ and/or L₂ consist of the amino acid sequence of 298, 299, or 302. In some embodiments, L₁ and L₂ each comprise the amino acid sequence of SEQ ID NO: 298, 299, or 302. In some embodiments, L₁ and L₂ each consist of the amino acid sequence of SEQ ID NO: 298, 299, or 302.

TABLE 4
Sequences of Exemplary Peptide Linkers

| | SEQ ID | | |
|---|---|---|---|
| Linker | NO | Length | Amino Acid Sequence |
| (GS)₁₀ | 292 | 20 | GSGSGSGSGSGSGSGSGSGS |
| (GGS)₁₀ | 293 | 30 | GGSGGSGGSGGSGGSGGSGGSGGSGGSGGS |
| (GGGS)₁₀ | 294 | 40 | GGGSGGGSGGGSGGGSGGGSGGGSGGGSGGGSGGGS |
| | | | GGGS |
| (GGSG)₁₀ | 295 | 40 | GGSGGGSGGGSGGGSGGGSGGGSGGGSGGGSGGGSG |
| | | | GGSG |
| (GGSGG)₁₀ | 296 | 50 | GGSGGGGSGGGGSGGGGSGGGGSGGGGSGGGGSGG |
| | | | GGSGGGGSGGGGSGG |
| (GGGGS)₁₀ | 297 | 50 | GGGGSGGGGSGGGGSGGGGSGGGGSGGGGSGGGGS |
| | | | GGGGSGGGGSGGGGS |
| (GGGGS)₄ | 298 | 20 | GGGGSGGGGSGGGGSGGGGS |
| (GGGGS)₃ | 299 | 15 | GGGGSGGGGSGGGGS |
| (GGGGS)₂₀ | 300 | 100 | GGGGSGGGGSGGGGSGGGGSGGGGSGGGGSGGGGS |
| | | | GGGGSGGGGSGGGGSGGGGSGGGGSGGGGSGGGGS |
| | | | GGGGSGGGGSGGGGSGGGGSGGGGSGGGGS |
| (GGSGG)₂₀ | 301 | 100 | GGSGGGGSGGGGSGGGGSGGGGSGGGGSGGGGSGG |
| | | | GGSGGGGSGGGGSGGGGSGGGGSGGGGSGGGGSGG |
| | | | GGSGGGGSGGGGSGGGGSGGGGSGGGGSGG |
| Assymetrical | 302 | 9 | GGGGSGGGS |
| linker | | | |

A linker, such as a peptide linker disclosed herein, can also be used to connect the VH and VL of an scFv, as mentioned in subsection C above titled “Construct Formats.”

In certain embodiments, the multi-specific binding protein further comprises a tag peptide, for example, a Flag tag, a 6×His tag, or 10×His tag (HHHHHHHHHH, SEQ ID NO: 711). Such tag peptides are useful for purifying the multi-specific binding protein. In certain embodiments, the tag peptide (e.g., the 10×His tag) is positioned at the C-terminus of the multi-specific binding protein. In certain embodiments, the tag peptide (e.g., the 10×His tag) is positioned at the N-terminus of the multi-specific binding protein.

### E. Binding Affinity

In certain embodiments, the multi-specific binding protein binds CD19 (e.g., human CD19), CD3 (e.g., human CD3 and/or Macaca CD3), and/or serum albumin (e.g., HSA) with a K_{D} in the range of about 0.1 nM-about 100 μM. The K_{D} can be measured by a method known in the art, such as by SPR or by flow cytometry as described in Example 1 or 6 below.

In certain embodiments, the multi-specific binding protein binds CD19, CD3, and/or serum albumin with a K_{D} lower than or equal to (i.e., binding stronger than or equal to) 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, or 10 pM. For example, in certain embodiments, the multi-specific binding protein binds CD19, CD3, and/or serum albumin with a K_{D} in the range of about 10 pM-about 1 nM, about 10 pM-about 0.9 nM, about 10 pM-about 0.8 nM, about 10 pM-about 0.7 nM, about 10 pM-about 0.6 nM, about 10 pM nM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, or about 9 nM-about 10 nM.

In certain embodiments, the multi-specific binding protein binds CD19, CD3, and/or serum albumin with a K_{D} greater than or equal to (i.e., binding weaker than or equal to) 10 nM, 20 nM, 30 nM, 40 nM, 50 nM, 60 nM, 70 nM, 80 nM, 90 nM, or 100 nM. In certain embodiments, the multi-specific binding protein binds CD19, CD3, and/or serum albumin with a K_{D} in the range of about 10 nM-about 1000 nM, about 10 nM-about 900 nM, about 10 nM-about 800 nM, about 10 nM-about 700 nM, about 10 nM-about 600 nM, about 10 nM-about 500 nM, about 10 nM-about 400 nM, about 10 nM-about 300 nM, about 10 nM-about 200 nM, about 10 nM-about 100 nM, about 10 nM-about 50 nM, about 50 nM-about 1000 nM, about 100 nM-about 1000 nM, about 200 nM-about 1000 nM, about 300 nM-about 1000 nM, about 400 nM-about 1000 nM, about 500 nM-about 1000 nM, about 600 nM-about 1000 nM, about 700 nM-about 1000 nM, about 800 nM-about 1000 nM, or about 900 nM-about 1000 nM.

In certain embodiments, the K_{D} of binding to CD19 or CD3 is measured in the absence of serum albumin (e.g., HSA). In certain embodiments, the K_{D} of binding to CD19 or CD3 is measured in substantial absence of serum albumin (e.g., HSA). In certain embodiments, the K_{D} of binding to CD19 or CD3 is measured in the presence of serum albumin (e.g., HSA), for example, in the presence of about 10 mg/mL, 15 mg/mL, 20 mg/mL, 25 mg/mL, 30 mg/mL, 35 mg/mL, 40 mg/mL, 45 mg/mL, or 50 mg/mL serum albumin (e.g., HSA).

In certain embodiments, the multi-specific binding protein of the present disclosure binds CD19, CD3, and/or serum albumin with a similar K_{D} value to that of the respective antigen-binding site alone or a monoclonal antibody having the same antigen-binding site. In certain embodiments, the K_{D} value of the multi-specific binding protein to CD19, CD3, and/or serum albumin is increased by no more than 1.5 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, or 50 fold compared to that of the respective antigen-binding site alone or a monoclonal antibody having the same antigen-binding site.

In certain embodiments, the multi-specific binding protein of the present disclosure binds CD19 and/or CD3 with a similar K_{D} value in the presence of serum albumin to that in the absence or substantial absence of serum albumin. In certain embodiments, the K_{D} value of the multi-specific binding protein for binding CD19 and/or CD3 in the presence of serum albumin is increased by no more than 1.5 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, or 50 fold compared to that in the absence or substantial absence of serum albumin.

### F. Therapeutic Activities

The multi-specific binding protein disclosed herein is designed to simultaneously bind B cells and T cells. Recruitment of T cells facilitates lysis of the B cells involving cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO2007042261A2. Accordingly, binding of the multi-specific binding proteins to the target B cells destroys the target cells and/or impairs the progression of B cell related diseases.

Cytotoxicity mediated by multi-specific binding proteins of the invention can be measured in various ways in vitro. Effector cells can be e.g., stimulated enriched (human) CD8 positive T cells or unstimulated (human) peripheral blood mononuclear cells (PBMC). If the target cells are of macaque origin or express or are transfected with macaque target cell surface antigen which is bound by the first domain, the effector cells should also be of macaque origin such as a macaque T cell line, e.g., 4119LnPx. The target cells should express the protein targeted by the first antigen-binding site, e.g., CD19, HER2, BCMA, CD33, or EGFR. In some embodiments, the target cells should express CD19, e.g., human or macaque CD19.

In some embodiments, the target cells can be a cell line (such as CHO) which is stably or transiently transfected with CD19. Alternatively, in some embodiments, the target cells can be a cell line naturally expressing CD19, such as B lymphocytes. The effector to target cell (E:T) ratio is usually about 10:1, but can also vary. Killing of the target cells can be measured in a ⁵¹Cr-release assay (incubation time of about 18 hours) or in a in a FACS-based cytotoxicity assay (incubation time of about 48 hours). Other methods of measuring cell death are well-known to the skilled person, such as MTT or MTS assays, ATP-based assays including bioluminescent assays, the sulforhodamine B (SRB) assay, WST assay, clonogenic assay and the ECIS technology.

In certain embodiments, the cytotoxic activity mediated by the multi-specific binding protein disclosed herein is measured in a cell-based cytotoxicity assay described above. It is represented by the EC₅₀ value, which corresponds to the half maximal effective concentration (concentration of the multi-specific binding protein which induces a cytotoxic response halfway between the baseline and maximum). In certain embodiments, the EC₅₀ value of the multi-specific binding proteins is ≤5000 pM, for example, ≤4000 pM, ≤3000 pM, ≤2000 pM, ≤1000 pM, ≤500 pM, ≤400 pM, ≤300 pM, ≤200 pM, ≤100 pM, ≤50 pM, ≤20 pM, ≤10 pM, ≤5 pM, ≤4 pM, ≤3 pM, ≤2 pM, or ≤1 pM.

It is understood that an EC₅₀ value is generally lower when stimulated/enriched CD8⁺ T cells are used as effector cells, compared with unstimulated PBMC. It is further understood that the EC₅₀ value is generally lower when the target cells express a high level of the target cell surface antigen compared with a low level of the target antigen. For example, when stimulated/enriched human CD8⁺ T cells are used as effector cells (and either target cell surface antigen transfected cells such as CHO cells or target cell surface antigen positive human cell lines are used as target cells), the EC₅₀ value of multi-specific binding protein is ≤1000 pM, for example, ≤500 pM, ≤250 pM, ≤100 pM, ≤50 pM, ≤10 pM, or ≤5 pM. When human PBMCs are used as effector cells, the EC₅₀ value of the multi-specific binding protein is ≤5000 pM, for example, ≤4000 pM, ≤2000 pM, ≤1000 pM, ≤500 pM, ≤200 pM, ≤150 pM, 100 pM, X50 pM, ≤10 pM, or ≤5 pM. When a macaque T cell line such as LnPx4119 is used as effector cells, and a macaque target cell surface antigen transfected cell line such as CHO cells is used as target cell line, the EC₅₀ value of the multi-specific binding protein is ≤2000 pM, for example, ≤1500 pM, ≤1000 pM, ≤500 pM, ≤300 pM, ≤250 pM, ≤100 pM, ≤50 pM, ≤10 pM, or ≤5 pM.

Accordingly, in certain embodiments, the EC₅₀ value is measured using stimulated/enriched human CD8⁺ T cells as effector cells. In certain embodiments, the EC₅₀ value is measured using human PBMCs as effector cells. In certain embodiments, the EC₅₀ value is measured using a macaque T cell line such as LnPx4119 as effector cells and cells (e.g., CHO cells) engineered to express macaque CD19 as target cells.

In certain embodiments, the multi-specific binding protein of the present invention does not induce or mediate lysis of cells that do not express CD19. The term “does not induce lysis” or “does not mediate lysis,” or grammatical equivalents thereof, means that the multi-specific binding protein, at a concentration of up to 500 nM, does not induce or mediate lysis of more than 30%, for example, no more than 30%, 25%, 20%, 15%, 10%, 9%, 8%, 7%, 6% or 5% of cells that do not express CD19, whereby lysis of a cell line that expresses CD19 is set to be 100%.

In certain embodiments, a multi-specific binding protein disclosed herein is more effective in killing CD19-expressing cells (e.g., cancer cells) than the corresponding respective anti-CD19 or anti-CD3 monoclonal antibody at the same molar concentration. In certain embodiments, the multi-specific binding protein is more effective in killing CD19-expressing cells (e.g., cancer cells) than a combination of the corresponding respective anti-CD19 and anti-CD3 monoclonal antibodies each at the same molar concentration.

The cytotoxic activity of the multi-specific binding protein can be measured in the presence or absence of serum albumin (e.g., HSA). In certain embodiments, the cytotoxic activity disclosed above is measured in the absence of serum albumin (e.g., HSA). In certain embodiments, the cytotoxic activity disclosed above is measured in substantial absence of serum albumin (e.g., HSA). In certain embodiments, the cytotoxic activity disclosed above is measured in the presence of serum albumin (e.g., HSA), for example, in the presence of about 10 mg/mL, 15 mg/mL, 20 mg/mL, 25 mg/mL, 30 mg/mL, 35 mg/mL, 40 mg/mL, 45 mg/mL, or 50 mg/mL serum albumin (e.g., HSA).

In certain embodiments, the multi-specific binding protein of the present disclosure kills CD19-expressing cells with a similar EC₅₀ value in the presence of serum albumin to that in the absence or substantial absence of serum albumin. In certain embodiments, the EC₅₀ value of the multi-specific binding protein for killing CD19-expressing cells in the presence of serum albumin is increased by no more than 1.5 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, or 50 fold compared to that in the absence or substantial absence of serum albumin. It is understood that the presence of serum albumin (e.g., about 10 mg/mL, 15 mg/mL, 20 mg/mL, 25 mg/mL, 30 mg/mL, 35 mg/mL, 40 mg/mL, 45 mg/mL, or 50 mg/mL serum albumin) may also alter the EC₅₀ value of a multi-specific binding protein nonspecifically. The nonspecific effect can be assessed by comparing the EC₅₀ values of a control protein, which does not contain a serum albumin binding domain, in the presence and absence of serum albumin. In certain embodiments, the fold change is offset by the nonspecific effect of serum albumin on a control protein, such as a bispecific protein that binds CD19 and CD3.

### G. Construct Size

In certain embodiments, the molecular weight of the multi-specific binding protein is from about 40 kD to about 100 kD. In certain embodiments, the molecular weight of the multi-specific binding protein is at least 60 kD, at least 65 kD, at least 70 kD, at least 75 kD, at least 80 kD, at least 85 kD, at least 90 kD, or at least 95 kD. It is understood that smaller size generally contributes to faster diffusion and tissue penetration, but size reduction may not be as critical for the purpose of treating the indications with substantial presence of target cells (e.g., cancer cells) in the blood.

In certain embodiments, the molecular weight of the multi-specific binding protein is from about 40 kD to about 90 kD, from about 40 kD to about 80 kD, from about 40 kD to about 70 kD, from about 40 kD to about 60 kD, from about 40 kD to about 50 kD, from about 50 kD to about 100 kD, from about 50 kD to about 90 kD, from about 50 kD to about 80 kD, from about 50 kD to about 70 kD, from about 50 kD to about 60 kD, from about 60 kD to about 100 kD, from about 60 kD to about 90 kD, from about 60 kD to about 80 kD, from about 60 kD to about 70 kD, from about 65 kD to about 100 kD, from about 65 kD to about 90 kD, from about 65 kD to about 80 kD, from about 65 kD to about 70 kD, from about 70 kD to about 100 kD, from about 70 kD to about 90 kD, from about 70 kD to about 80 kD, from about 80 kD to about 100 kD, from about 80 kD to about 90 kD, or from about 90 kD to about 100 kD. In certain embodiments, the multi-specific binding protein is lower than 40 kD. In certain embodiments, the multi-specific binding protein is about 50 kD-about 90 kD, about 50 kD-about 80 kD, about 50 kD-about 70 kD, about 50 kD-about 60 kD, about 60 kD-about 90 kD, about 60 kD-about 80 kD, about 60 kD-about 70 kD, about 65 kD-about 90 kD, about 65 kD-about 80 kD, about 65 kD-about 70 kD, about 70 kD-about 90 kD, or about 70 kD-about 80 kD.

### H. Serum Half-Life

Fusion proteins have been developed to increase the in vivo half-life of a small protein, particularly an antibody fragment. For example, fusion with a heterodimeric antibody Fc region, such as an Fc with one or more mutations that extend the in vivo half-life, is described in U.S. Patent Application Publication Nos. US20140302037A1, US20140308285A1, and PCT Publication Nos. WO2014144722A2, WO2014151910A1 and WO2015048272A1. An alternative strategy is fusion with human serum albumin (HSA) or an HSA-binding peptide (see, e.g., PCT Publication Nos. WO2013128027A1 and WO2014140358A1). The neonatal Fc receptor (FcRn) appears to be involved in prolonging the life-span of albumin in circulation (see, Chaudhury et al. (2003) J. Exp. Med., 3: 315-22). Albumin and IgG bind noncooperatively to distinct sites of FcRn and form a tri-molecular (see id.). Binding of human FcRn to HSA and to human IgG is pH dependent, stronger at acidic pH and weaker at neutral or physiological pH (see id.). This observation suggests that proteins and protein complexes containing albumin, similar to those containing IgG (particularly Fc), are protected from degradation through pH-sensitive interaction with FcRn (see id.). Using surface plasmon resonance (SPR) to measure the capacity of individual HSA domains to bind immobilized soluble human FcRn, it has been shown that FcRn and albumin interact via the D-III domain of albumin in a pH-dependent manner, on a site distinct from the IgG binding site (see, Chaudhury et al. (2006) Biochemistry 45:4983-90 and PCT Publication No. WO2008068280A1).

The present disclosure provides multi-specific binding proteins with extended half-life. In certain embodiments, the multi-specific binding protein has a serum half-life of at least 24, 36, 48, 60, 72, 84, or 96 hours. In certain embodiments, the multi-specific binding protein has a serum half-life of at least about 50 hours. In certain embodiments, the multi-specific binding protein has a serum half-life of at least about 100 hours. Methods of measuring serum half-life are known in the art, and exemplary methods are described in Example 5. In certain embodiments, the serum half-life is measured in a non-human primate. In certain embodiments, the serum half-life is measured in human.

In certain embodiments, 50 hours after intravenous administration to a subject, the serum concentration of the multi-specific binding protein is at least 10%, at least 20%, at least 30%, at least 40%, at least 50%, at least 60%, at least 70%, at least 80%, or at least 90% of the serum concentration of the multi-specific binding protein 1 hour after the administration in said subject.

In certain embodiments, the multi-specific binding protein has a serum half-life that is at least 20% longer than a control multi-specific binding protein, wherein the control multi-specific binding protein includes a first domain identical to the first antigen-binding site of the multi-specific binding protein, a second domain identical to the second antigen-binding site of the multi-specific binding protein, but not a third domain identical or substantially identical to the third antigen-binding site of the multi-specific binding protein. In certain embodiments, the control multi-specific binding protein is identical to the multi-specific binding protein but for the absence of the third antigen-binding site. In certain embodiments, the serum half-life of the multi-specific binding protein is at least 30%, at least 40%, at least 50%, at least 60%, at least 70%, at least 80%, or at least 90% longer than the serum half-life of the control multi-specific binding protein. In certain embodiments, the serum half-life of the multi-specific binding protein is longer than the serum half-life of the control multi-specific binding protein by at least 2 fold, at least 3 fold, at least 4 fold, at least 5 fold, at least 6 fold, at least 7 fold, at least 8 fold, at least 9 fold, or at least 10 fold.

### III. Methods of Preparation

The antibodies and multi-specific binding proteins described above can be made using recombinant DNA technology well known to a skilled person in the art. For example, one or more isolated polynucleotides encoding the antibody or the multi-specific binding protein can be ligated to other appropriate nucleotide sequences, including, for example, constant region coding sequences, and expression control sequences, to produce conventional gene expression constructs (i.e., expression vectors) encoding the desired antibodies or multi-specific binding proteins. Production of defined gene constructs is within routine skill in the art.

Nucleic acids encoding desired antibodies or multi-specific binding proteins can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the antibodies or multi-specific binding proteins.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The antibodies or multi-specific binding protein disclosed herein may comprise a single polypeptide chain. In this instance, a host cell can be transfected with a single vector expressing the polypeptide (e.g., containing an expression control sequence operably linked to a nucleotide sequence encoding the polypeptide). Alternatively, the antibodies or multi-specific binding proteins disclosed herein may comprise two or more polypeptides. In this instance, a host cell can be co-transfected with more than one expression vector, for example, one expression vector expressing each polypeptide. A host cell can also be transfected with a single expression vector that expresses the two or more polypeptides. For example, the coding sequences of the two or more polypeptides can be operably linked to different expression control sequences (e.g., promoter, enhancer, and/or internal ribosome entry site (IRES)). The coding sequences of the two or more polypeptides can also be separated by a ribosomal skipping sequence or self-cleaving sequence, such as a 2A peptide.

In certain embodiments, in order to express an antibody or multi-specific binding protein, an N-terminal signal sequence is included in the protein construct. Exemplary N-terminal signal sequences include signal sequences from interleukin-2, CD-5, IgG kappa light chain, trypsinogen, serum albumin, and prolactin.

After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of the antibodies or multi-specific binding proteins.

The antibodies or multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### IV. Pharmaceutical Compositions

The present disclosure also features pharmaceutical compositions that contain a therapeutically effective amount of the antibodies or multi-specific binding proteins described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing an antibody or a multi-specific binding protein disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a recombinant human sialidase, a recombinant human sialidase fusion protein, or an antibody conjugate disclosed herein is administered by IV infusion. In certain embodiments, a recombinant human sialidase, a recombinant human sialidase fusion protein, or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous drug delivery formulation may be contained in a syringe, pen, or bag. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1,000 mg/vial. In certain embodiments, the formulation may be a liquid formulation and stored as about 600 mg/vial. In certain embodiments, the formulation may be a liquid formulation and stored as about 250 mg/vial.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the antibody or multi-specific binding protein, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The antibody or multi-specific binding protein may be lyophilized to produce a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5. In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5,000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

### V. Therapeutic Applications

It is contemplated that the antibodies or multi-specific binding proteins can be used either alone or in combination with other therapeutic agents.

### A. Indications

The present disclosure provides methods for the treatment or amelioration of a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease in a subject in need thereof, the method comprising administration of a multi-specific binding protein or an antibody disclosed herein. In certain embodiments, the disease is associated with expression or overexpression of the target protein expressed on the target cell.

In certain embodiments, the cancer to be treated is non-Hodgkin's lymphoma, such as a B-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, chronic lymphocytic leukemia, or primary central nervous system lymphoma. In certain other embodiments, the cancer to be treated is multiple myeloma. In certain other embodiments, the cancer to be treated is acute lymphoblastic leukemia (ALL). In certain embodiments, the ALL is relapsed/refractory adult and pediatric ALL.

### B. Combination Therapies

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In one aspect, the present disclosure provides a method of treating a subject by the administration of a second therapeutic agent in combination with one or more of the multi-specific binding proteins and/or antibodies that bind CD19 disclosed herein.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with (i) a PD-1 or PD-L1 inhibitor, e.g., a PD-1 or PD-L1 inhibitor disclosed herein, and (ii) CTLA-4 inhibitor, e.g., a CTLA-4 inhibitor disclosed herein.

In certain embodiments, a method or composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

It is understood that the antibody or multi-specific binding protein disclosed herein, which is designed to activate T lymphocytes, may cause side effects such as neurotoxicity. Accordingly, in certain embodiments, the second therapeutic agent that can be used in combination with the antibody or multi-specific binding protein comprises an agent that mitigates a side effect of the antibody or multi-specific binding protein, e.g., reduces neurotoxicity. In certain embodiments, the second therapeutic agent inhibits T cell trafficking, for example, reduces or inhibits immune cells from crossing the blood-brain barrier. Non-limiting examples of such therapeutic agents include antagonists (e.g., antagonistic antibodies) of adhesion molecules on immune cells (e.g., α4 integrin), such as natalizumab. In certain embodiments, the second therapeutic agent increases the internalization of a sphingosine-1-phosphate (SIP) receptor (e.g., S1PR1 or S1PR5), such as fingolimod or ozanimod. In certain embodiments, the second therapeutic agent is a nitric oxide synthase (NOS) inhibitor, such as ronopterin, cindunistat, A-84643, ONO-1714, L-NOARG, NCX-456, VAS-2381, GW-273629, NXN-462, CKD-712, K_{D}-7040, or guanidinoethyldisulfide. In certain embodiments, the second therapeutic agent is an antagonist of CSF1 or CSF1R, such as pexidartinib, emactuzumab, cabiralizumab, LY-3022855, JNJ-40346527, or MCS110. Additional non-limiting examples of the second therapeutic agents include pentosan polysulfate, minocycline, anti-ICAM-1 antibodies, anti-P-selectin antibodies, anti-CD11a antibodies, anti-CD162 antibodies, and anti-IL-6R antibodies (e.g., tocilizumab).

The amount of the antibody or multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, an antibody or multi-specific binding protein may be administered during a time when the additional therapeutic agent(s) exerts its prophylactic or therapeutic effect, or vice versa.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and is not intended to limit the invention.

### Example 1. Characterization of New Anti-Serum Albumin Antibodies

This example describes new anti-serum albumin antibodies CNG-HSA-101 to -120. The amino acid sequences of these antibodies are provided in Table 1 above.

CNG-HSA-101 to -120 were optimized from parental antibodies CNG-HSA-1, a single domain antibody, by introducing diversities into the heavy chain variable region, generating random mutations by error prone PCR, and shuffling the VH fragments. The antibody clones were selected for improved binding affinity to biotinylated human serum albumin relative to the parental antibody. Additionally, a thermal selection pressure was employed for the VH shuffle optimization cycle. Thermal selection pressures were applied by incubating the libraries at various temperatures and then selecting for antibodies that retained antigen binding following thermal incubation. The selected antibodies were then produced from yeast cells and purified using a protein A column.

The binding affinity of the antibodies to isolated serum albumin was measured by surface plasmon resonance using a ForteBio Octet HTX system as previously described (see, e.g., Estep et al, High throughput solution-based measurement of antibody-antigen affinity and epitope binning Mabs 5(2), 270-278 (2013)). Briefly, ForteBio affinity measurements were performed by loading the heavy chain antibodies (HCAbs) on-line onto AHC sensors. The sensors were equilibrated off-line in assay buffer for 30 minutes and then monitored on-line for 60 seconds for baseline establishment. The sensors with loaded HCAbs were exposed to 100 nM human serum albumin for 3 minutes, and subsequently transferred to assay buffer for 3 minutes for off-rate measurement. All kinetics were analyzed using the 1:1 binding model.

The melting temperature (Tm) of the VHH fragments were measured by dynamic scanning fluorimetry (DSF). Briefly, 10 μL of 20× Sypro Orange dye is added to 20 μL of 0.2-1 mg/mL HCAb. A BioRad CFX96 RT PCR machine is used to raise the sample plate temperature from 40° to 95° C. at 0.5° C. increments, with 2 minutes equilibration at each temperature. The negative of the first derivative for the raw data is used to extract Tm.

TABLE 5
Binding of Anti-Serum Albumin Antibodies to Serum Albumin and Protein A

| | Affinity to | | | | | | Ratio | | | |
|---|---|---|---|---|---|---|---|---|---|---|
| | Human | | Affinity to | | Affinity to | | of | | | |
| | Serum | | Cynomolgus | | Mouse Serum | | MSA | | | |
| | Albumin | | Serum | | Albumin | | -K_{D} | Affinity to | | |
| | (HSA) | | Albumin | | (MSA) | | to | Protein A | | Melting |
| | K_{D} | k_{off} | K_{D} | k_{off} | K_{D} | k_{off} | HSA- | K_{d} | koff | Temperature |
| Construct | (M) | (1/s) | (M) | (1/s) | (M) | (1/s) | K_{D} | (M) | (1/s) | (° C.) |
| CNG- | 1.19E−08 | 1.77E−03 | 9.65E−09 | 1.39E−03 | 1.72E−07 | 2.16E−02 | 14.47 | 2.56E−09 | 4.74E−04 | 55.5 |
| HSA-1 | | | | | | | | | | |
| CNG- | 5.07E−09 | 7.16E−04 | 3.58E−09 | 5.32E−04 | 1.61E−08 | 3.41E−03 | 3.17 | 1.84E−09 | 2.00E−04 | 65.5 |
| HSA-101 | | | | | | | | | | |
| CNG- | 4.89E−09 | 7.48E−04 | 3.65E−09 | 5.68E−04 | 4.14E−08 | 7.38E−03 | 8.46 | 1.70E−09 | 2.00E−04 | 66.5 |
| HSA-102 | | | | | | | | | | |
| CNG- | 4.89E−09 | 7.42E−04 | 3.62E−09 | 5.41E−04 | 1.51E−08 | 3.24E−03 | 3.09 | 2.02E−09 | 2.21E−04 | 66.0 |
| HSA-103 | | | | | | | | | | |
| CNG- | 7.71E−09 | 1.12E−03 | 6.10E−09 | 8.74E−04 | 5.06E−08 | 1.05E−02 | 6.57 | 1.70E−09 | 2.00E−04 | 63.5 |
| HSA-104 | | | | | | | | | | |
| CNG- | 1.52E−08 | 2.00E−03 | 1.27E−08 | 1.63E−03 | 1.54E−07 | 2.06E−02 | 10.12 | 2.76E−09 | 2.97E−04 | 64.0 |
| HSA-105 | | | | | | | | | | |
| CNG- | 1.70E−08 | 2.97E−03 | 1.44E−08 | 2.38E−03 | 5.78E−08 | 1.24E−02 | 3.41 | N.B.¹ | | 65.0 |
| HSA-106 | | | | | | | | | | |
| CNG- | 1.68E−08 | 2.38E−03 | 1.33E−08 | 1.82E−03 | 4.56E−08 | 8.51E−03 | 2.72 | 3.09E−09 | 3.33E−04 | 59.0 |
| HSA-107 | | | | | | | | | | |
| CNG- | 6.19E−09 | 9.58E−04 | 5.27E−09 | 7.94E−04 | 8.06E−09 | 1.85E−03 | 1.30 | 2.94E−09 | 2.94E−04 | 64.0 |
| HSA-108 | | | | | | | | | | |
| CNG- | 3.50E−09 | 5.66E−04 | 3.10E−09 | 5.03E−04 | 1.19E−08 | 2.84E−03 | 3.40 | 1.78E−09 | 2.00E−04 | 63.5 |
| HSA-109 | | | | | | | | | | |
| CNG- | 5.70E−09 | 8.93E−04 | 5.20E−09 | 7.59E−04 | 3.89E−08 | 8.38E−03 | 6.82 | 3.67E−09 | 3.78E−04 | 57.5 |
| HSA-110 | | | | | | | | | | |
| CNG- | 8.35E−09 | 1.17E−03 | 6.13E−09 | 8.84E−04 | 1.02E−08 | 2.31E−03 | 1.23 | 2.84E−09 | 2.00E−04 | 59.0 |
| HSA-111 | | | | | | | | | | |
| CNG- | 8.45E−09 | 1.36E−03 | 7.43E−09 | 1.15E−03 | 6.67E−08 | 1.30E−02 | 7.90 | 3.44E−09 | 3.75E−04 | 54.5 |
| HSA-112 | | | | | | | | | | |
| CNG- | 1.54E−08 | 2.14E−03 | 1.21E−08 | 1.70E−03 | 5.20E−08 | 1.01E−02 | 3.38 | 1.70E−09 | 2.00E−04 | 62.5 |
| HSA-113 | | | | | | | | | | |
| CNG- | 1.47E−08 | 2.21E−03 | 1.20E−08 | 1.73E−03 | 1.75E−08 | 3.80E−03 | 1.20 | N.D.² | | 51.0 |
| HSA-114 | | | | | | | | | | |
| CNG- | 2.96E−09 | 4.41E−04 | 3.37E−09 | 6.18E−04 | 6.43E−09 | 1.32E−03 | 2.17 | N.D. | | 56.0 |
| HSA-115 | | | | | | | | | | |
| CNG- | 4.65E−09 | 6.99E−04 | 3.85E−09 | 5.97E−04 | 4.56E−09 | 1.08E−03 | 0.98 | 1.75E−09 | 2.00E−04 | 64.0 |
| HSA-116 | | | | | | | | | | |
| CNG- | 5.56E−09 | 7.93E−04 | 4.05E−09 | 6.08E−04 | 4.48E−08 | 8.77E−03 | 8.07 | 1.78E−09 | 2.00E−04 | 60.5 |
| HSA-117 | | | | | | | | | | |

| | | | | | | | | | |
|---|---|---|---|---|---|---|---|---|---|
| CNG- | 9.36E−09 | 1.30E−03 | 6.65E−09 | 1.02E−03 | 1.20E−08 | 2.74E−03 | 1.28 | N.D. | 58.0 |
| HSA-118 | | | | | | | | | |
| CNG- | 7.36E−09 | 1.09E−03 | 5.73E−09 | 8.64E−04 | 4.11E−08 | 8.54E−03 | 5.59 | N.D. | 53.5 |
| HSA-119 | | | | | | | | | |
| CNG- | 3.15E−09 | 5.07E−04 | 2.60E−09 | 4.22E−04 | 4.32E−09 | 1.07E−03 | 1.37 | N.D. | 66.0 |
| HSA-120 | | | | | | | | | |

¹N.B. means no binding was detected under the conditions of this assay.
²N.D. means not determined.

As shown in Table 5, CNG-HSA-101 to -120 showed higher binding affinity to human serum albumin, cynomolgus serum albumin, mouse serum albumin, and/or protein A than CNG-HSA-1. In particular, all these antibodies bound mouse serum albumin with lower K_{D} values than CNG-HSA-1. CNG-HSA-101, CNG-HSA-102, CNG-HSA-103, CNG-HSA-104, CNG-HSA-108, CNG-HSA-109, CNG-HSA-110, CNG-HSA-111, CNG-HSA-112, CNG-HSA-115, CNG-HSA-116, CNG-HSA-117, CNG-HSA-118, CNG-HSA-119, and CNG-HSA-120 bound human serum albumin and cynomolgus serum albumin with lower K_{D} values than CNG-HSA-1. CNG-HSA-101, CNG-HSA-103, CNG-HSA-106, CNG-HSA-107, CNG-HSA-108, CNG-HSA-109, CNG-HSA-111, CNG-HSA-113, CNG-HSA-114, CNG-HSA-115, CNG-HSA-116, CNG-HSA-118, and CNG-HSA-120 bound mouse serum albumin with a K_{D} value less than 4-fold higher than the K_{D} with which the same antibody bound human serum albumin. CNG-HSA-101, CNG-HSA-102, CNG-HSA-104, CNG-HSA-109, CNG-HSA-113, CNG-HSA-116, and CNG-HSA-117 bound protein A with lower K_{D} values than CNG-HSA-1. CNG-HSA-101, CNG-HSA-102, CNG-HSA-103, CNG-HSA-104, CNG-HSA-105, CNG-HSA-106, CNG-HSA-108, CNG-HSA-109, CNG-HSA-113, CNG-HSA-116, CNG-HSA-117, and CNG-HSA-120 showed a melting temperature greater than or equal to 60° C., among which CNG-HSA-101, CNG-HSA-102, CNG-HSA-103, CNG-HSA-106, and CNG-HSA-120 showed a melting temperature greater than or equal to 65° C.

### Example 2. Production of Multi-Specific Binding Proteins

This example describes the production and purification of multi-specific binding proteins.

Nucleic acids encoding single-chain multi-specific binding proteins (see Table 6) were constructed and codon optimized for expression in human cells and cloned into a mammalian expression vector following standard procedures. Following sequence verification, the expression vectors, in the form of plasmids, were prepared in sufficient quantity for transfection using Plasmid Plus purification kits (Qiagen). Human embryonic kidney 293 (HEK 293) cells were passaged to appropriate density for transient transfection. Cells were transiently transfected with the expression vectors and cultured for six days.

The amino acid sequences of the various multi-specific binding proteins are summarized in Table 6. Constructs tAb0027 to tAb0032 each contained an anti-CD19 scFv having the amino acid sequence set forth in SEQ ID NO: 9, an anti-CD3 scFv having the amino acid sequence set forth in SEQ ID NO: 105, and an anti-HSA sdAb having the amino acid sequence set forth in SEQ ID NO: 121. Constructs tAb0033 to tAb0038 each contained an anti-CD19 scFv having the amino acid sequence set forth in SEQ ID NO: 18, an anti-CD3 scFv having the amino acid sequence set forth in SEQ ID NO: 105, and an anti-HSA sdAb having the amino acid sequence set forth in SEQ ID NO: 121.

TABLE 6
Exemplary Multi-specific Binding Proteins

| Construct | Format | Amino Acid Sequence |
|---|---|---|
| tAb0027 | CD19:CD3: | QVQLVQSGAEVKKPGASVKVSCKASGYTFTDYIMHWVRQAPGQ |
| | HSA | GLEWMGYINPYNDGSKYTEKFQGRVTMTSDTSISTAYMELSRLRS |
| | | DDTAVYYCARGTYYYGPQLFDYWGQGTTVTVSSGGGGSGGGGS |
| | | GGGGSDIVMTQTPLSLSVTPGQPASISCKSSQSLETSTGTTYLNWY |
| | | LQKPGQSPQLLIYRVSKRFSGVPDRFSGSGSGTDFTLKISRVEAEDV |
| | | GVYYCLQLLEDPYTFGQGTKLEIKGGGGSGGGSDIVMTQSPDSLA |
| | | VSLGERATINCKSSQSLLNARTGKNYLAWYQQKPGQPPKLLIYWA |
| | | STRESGVPDRFSGSGSGTDFTLTISSLQAEDVAVYYCKQSYSRRTF |
| | | GGGTKVEIKGGGGSGGGGSGGGGSGGGGSQVQLVQSGAEVKKP |
| | | GASVKVSCKASGFNIKDYYMHWVRQAPGQRLEWMGWIDLENAN |
| | | TIYDAKFQGRVTITRDTSASTAYMELSSLRSEDTAVYYCARDAYG |
| | | RYFYDVWGQGTLVTVSSGGGGSGGGSEVQLVESGGGLVQPGNSL |
| | | RLSCAASGFTFSSFGMSWVRQAPGKGLEWVSSISGSGSDTLYADS |
| | | VKGRFTISRDNAKTTLYLQMNSLRPEDTAVYYCTIGGSLSRSSQGT |
| | | LVTVSSHHHHHHHHHH (SEQ ID NO: 303) |
| tAb0029 | CD3:CD19: | DIVMTQSPDSLAVSLGERATINCKSSQSLLNARTGKNYLAWYQQK |
| | HSA | PGQPPKLLIYWASTRESGVPDRFSGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSQVQLVQ |
| | | SGAEVKKPGASVKVSCKASGYTFTDYIMHWVRQAPGQGLEWMG |
| | | YINPYNDGSKYTEKFQGRVTMTSDTSISTAYMELSRLRSDDTAVY |
| | | YCARGTYYYGPQLFDYWGQGTTVTVSSGGGGSGGGGSGGGGSDI |
| | | VMTQTPLSLSVTPGQPASISCKSSQSLETSTGTTYLNWYLQKPGQS |
| | | PQLLIYRVSKRFSGVPDRFSGSGSGTDFTLKISRVEAEDVGVYYCL |
| | | QLLEDPYTFGQGTKLEIKGGGGSGGGSEVQLVESGGGLVQPGNSL |
| | | RLSCAASGFTFSSFGMSWVRQAPGKGLEWVSSISGSGSDTLYADS |
| | | VKGRFTISRDNAKTTLYLQMNSLRPEDTAVYYCTIGGSLSRSSQGT |
| | | LVTVSSHHHHHHHHHH (SEQ ID NO: 304) |
| tAb0030 | CD3:HSA: | DIVMTQSPDSLAVSLGERATINCKSSQSLLNARTGKNYLAWYQQK |
| | CD19 | PGQPPKLLIYWASTRESGVPDRFSGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSEVQLVE |
| | | SGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYC |
| | | TIGGSLSRSSQGTLVTVSSGGGGSGGGSQVQLVQSGAEVKKPGAS |
| | | VKVSCKASGYTFTDYIMHWVRQAPGQGLEWMGYINPYNDGSKY |
| | | TEKFQGRVTMTSDTSISTAYMELSRLRSDDTAVYYCARGTYYYGP |
| | | QLFDYWGQGTTVTVSSGGGGSGGGGSGGGGSDIVMTQTPLSLSV |
| | | TPGQPASISCKSSQSLETSTGTTYLNWYLQKPGQSPQLLIYRVSKRF |
| | | SGVPDRFSGSGSGTDFTLKISRVEAEDVGVYYCLQLLEDPYTFGQG |
| | | TKLEIKHHHHHHHHHH (SEQ ID NO: 305) |
| tAb0031 | HSA:CD3: | EVQLVESGGGLVQPGNSLRLSCAASGFTFSSFGMSWVRQAPGKGL |
| | CD19 | EWVSSISGSGSDTLYADSVKGRFTISRDNAKTTLYLQMNSLRPEDT |
| | | AVYYCTIGGSLSRSSQGTLVTVSSGGGGSGGGSDIVMTQSPDSLAV |
| | | SLGERATINCKSSQSLLNARTGKNYLAWYQQKPGQPPKLLIYWAS |
| | | TRESGVPDRFSGSGSGTDFTLTISSLQAEDVAVYYCKQSYSRRTFG |
| | | GGTKVEIKGGGGSGGGGSGGGGSGGGGSQVQLVQSGAEVKKPG |
| | | ASVKVSCKASGFNIKDYYMHWVRQAPGQRLEWMGWIDLENANT |
| | | IYDAKFQGRVTITRDTSASTAYMELSSLRSEDTAVYYCARDAYGR |
| | | YFYDVWGQGTLVTVSSGGGGSGGGSQVQLVQSGAEVKKPGASV |
| | | KVSCKASGYTFTDYIMHWVRQAPGQGLEWMGYINPYNDGSKYT |
| | | EKFQGRVTMTSDTSISTAYMELSRLRSDDTAVYYCARGTYYYGPQ |
| | | LFDYWGQGTTVTVSSGGGGSGGGGSGGGGSDIVMTQTPLSLSVTP |
| | | GQPASISCKSSQSLETSTGTTYLNWYLQKPGQSPQLLIYRVSKRFS |
| | | GVPDRFSGSGSGTDFTLKISRVEAEDVGVYYCLQLLEDPYTFGQG |
| | | TKLEIKHHHHHHHHHH (SEQ ID NO: 306) |
| tAb0032 | HSA:CD19 | EVQLVESGGGLVQPGNSLRLSCAASGFTFSSFGMSWVRQAPGKGL |
| | :CD3 | EWVSSISGSGSDTLYADSVKGRFTISRDNAKTTLYLQMNSLRPEDT |
| | | AVYYCTIGGSLSRSSQGTLVTVSSGGGGSGGGSQVQLVQSGAEVK |
| | | KPGASVKVSCKASGYTFTDYIMHWVRQAPGQGLEWMGYINPYN |
| | | DGSKYTEKFQGRVTMTSDTSISTAYMELSRLRSDDTAVYYCARGT |
| | | YYYGPQLFDYWGQGTTVTVSSGGGGSGGGGSGGGGSDIVMTQTP |
| | | LSLSVTPGQPASISCKSSQSLETSTGTTYLNWYLQKPGQSPQLLIYR |
| | | VSKRFSGVPDRFSGSGSGTDFTLKISRVEAEDVGVYYCLQLLEDPY |
| | | TFGQGTKLEIKGGGGSGGGSDIVMTQSPDSLAVSLGERATINCKSS |
| | | QSLLNARTGKNYLAWYQQKPGQPPKLLIYWASTRESGVPDRFSGS |
| | | GSGTDFTLTISSLQAEDVAVYYCKQSYSRRTFGGGTKVEIKGGGG |
| | | SGGGGSGGGGSGGGGSQVQLVQSGAEVKKPGASVKVSCKASGFN |
| | | IKDYYMHWVRQAPGQRLEWMGWIDLENANTIYDAKFQGRVTITR |
| | | DTSASTAYMELSSLRSEDTAVYYCARDAYGRYFYDVWGQGTLVT |
| | | VSSHHHHHHHHHH (SEQ ID NO: 307) |
| tAb0033 | CD19:CD3: | QVQLQESGPGLVKPSQTLSLTCTVSGGSISTSGMGVGWIRQHPGK |
| | HSA | GLEWIGHIWWDDDKRYNPALKSRVTISVDTSKNQFSLKLSSVTAA |
| | | DTAVYYCARMELWSYYFDYWGQGTLVTVSSGGGGSGGGGSGGG |
| | | GSEIVLTQSPATLSLSPGERATLSCSASSSVSYMHWYQQKPGQAPR |
| | | LLIYDTSKLASGIPARFSGSGSGTDFTLTISSLEPEDFAVYYCFQGSV |
| | | YPFTFGQGTKLEIKRGGGGSGGGSDIVMTQSPDSLAVSLGERATIN |
| | | CKSSQSLLNARTGKNYLAWYQQKPGQPPKLLIYWASTRESGVPD |
| | | RFSGSGSGTDFTLTISSLQAEDVAVYYCKQSYSRRTFGGGTKVEIK |
| | | GGGGSGGGGSGGGGSGGGGSQVQLVQSGAEVKKPGASVKVSCK |
| | | ASGFNIKDYYMHWVRQAPGQRLEWMGWIDLENANTIYDAKFQG |
| | | RVTITRDTSASTAYMELSSLRSEDTAVYYCARDAYGRYFYDVWG |
| | | QGTLVTVSSGGGGSGGGSEVQLVESGGGLVQPGNSLRLSCAASGF |
| | | TFSSFGMSWVRQAPGKGLEWVSSISGSGSDTLYADSVKGRFTISRD |
| | | NAKTTLYLQMNSLRPEDTAVYYCTIGGSLSRSSQGTLVTVSSHHH |
| | | HHHHHHH (SEQ ID NO: 308) |
| tAb0034 | CD19:HSA: | QVQLQESGPGLVKPSQTLSLTCTVSGGSISTSGMGVGWIRQHPGK |
| | CD3 | GLEWIGHIWWDDDKRYNPALKSRVTISVDTSKNQFSLKLSSVTAA |
| | | DTAVYYCARMELWSYYFDYWGQGTLVTVSSGGGGSGGGGSGGG |
| | | GSEIVLTQSPATLSLSPGERATLSCSASSSVSYMHWYQQKPGQAPR |
| | | LLIYDTSKLASGIPARFSGSGSGTDFTLTISSLEPEDFAVYYCFQGSV |
| | | YPFTFGQGTKLEIKRGGGGSGGGSEVQLVESGGGLVQPGNSLRLS |
| | | CAASGFTFSSFGMSWVRQAPGKGLEWVSSISGSGSDTLYADSVKG |
| | | RFTISRDNAKTTLYLQMNSLRPEDTAVYYCTIGGSLSRSSQGTLVT |
| | | VSSGGGGSGGGSDIVMTQSPDSLAVSLGERATINCKSSQSLLNART |
| | | GKNYLAWYQQKPGQPPKLLIYWASTRESGVPDRFSGSGSGTDFTL |
| | | TISSLQAEDVAVYYCKQSYSRRTFGGGTKVEIKGGGGSGGGGSGG |
| | | GGSGGGGSQVQLVQSGAEVKKPGASVKVSCKASGFNIKDYYMH |
| | | WVRQAPGQRLEWMGWIDLENANTIYDAKFQGRVTITRDTSASTA |
| | | YMELSSLRSEDTAVYYCARDAYGRYFYDVWGQGTLVTVSSHHH |
| | | HHHHHHH (SEQ ID NO: 309) |
| tAb0035 | CD3:CD19: | DIVMTQSPDSLAVSLGERATINCKSSQSLLNARTGKNYLAWYQQK |
| | HSA | PGQPPKLLIYWASTRESGVPDRFSGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSQVQLQE |
| | | SGPGLVKPSQTLSLTCTVSGGSISTSGMGVGWIRQHPGKGLEWIGH |
| | | IWWDDDKRYNPALKSRVTISVDTSKNQFSLKLSSVTAADTAVYYC |
| | | ARMELWSYYFDYWGQGTLVTVSSGGGGSGGGGSGGGGSEIVLTQ |
| | | SPATLSLSPGERATLSCSASSSVSYMHWYQQKPGQAPRLLIYDTSK |
| | | LASGIPARFSGSGSGTDFTLTISSLEPEDFAVYYCFQGSVYPFTFGQ |
| | | GTKLEIKRGGGGSGGGSEVQLVESGGGLVQPGNSLRLSCAASGFT |
| | | FSSFGMSWVRQAPGKGLEWVSSISGSGSDTLYADSVKGRFTISRD |
| | | NAKTTLYLQMNSLRPEDTAVYYCTIGGSLSRSSQGTLVTVSSHHH |
| | | HHHHHHH (SEQ ID NO: 310) |
| tAb0036 | CD3:HSA: | DIVMTQSPDSLAVSLGERATINCKSSQSLLNARTGKNYLAWYQQK |
| | CD19 | PGQPPKLLIYWASTRESGVPDRFSGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSEVQLVE |
| | | SGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYC |
| | | TIGGSLSRSSQGTLVTVSSGGGGSGGGSQVQLQESGPGLVKPSQTL |
| | | SLTCTVSGGSISTSGMGVGWIRQHPGKGLEWIGHIWWDDDKRYN |
| | | PALKSRVTISVDTSKNQFSLKLSSVTAADTAVYYCARMELWSYYF |
| | | DYWGQGTLVTVSSGGGGSGGGGSGGGGSEIVLTQSPATLSLSPGE |
| | | RATLSCSASSSVSYMHWYQQKPGQAPRLLIYDTSKLASGIPARFSG |
| | | SGSGTDFTLTISSLEPEDFAVYYCFQGSVYPFTFGQGTKLEIKRHHH |
| | | HHHHHHH (SEQ ID NO: 311) |
| tAb0037 | HSA:CD3: | EVQLVESGGGLVQPGNSLRLSCAASGFTFSSFGMSWVRQAPGKGL |
| | CD19 | EWVSSISGSGSDTLYADSVKGRFTISRDNAKTTLYLQMNSLRPEDT |
| | | AVYYCTIGGSLSRSSQGTLVTVSSGGGGSGGGSDIVMTQSPDSLAV |
| | | SLGERATINCKSSQSLLNARTGKNYLAWYQQKPGQPPKLLIYWAS |
| | | TRESGVPDRFSGSGSGTDFTLTISSLQAEDVAVYYCKQSYSRRTFG |
| | | GGTKVEIKGGGGSGGGGSGGGGSGGGGSQVQLVQSGAEVKKPG |
| | | ASVKVSCKASGFNIKDYYMHWVRQAPGQRLEWMGWIDLENANT |
| | | IYDAKFQGRVTITRDTSASTAYMELSSLRSEDTAVYYCARDAYGR |
| | | YFYDVWGQGTLVTVSSGGGGSGGGSQVQLQESGPGLVKPSQTLS |
| | | LTCTVSGGSISTSGMGVGWIRQHPGKGLEWIGHIWWDDDKRYNP |
| | | ALKSRVTISVDTSKNQFSLKLSSVTAADTAVYYCARMELWSYYFD |
| | | YWGQGTLVTVSSGGGGSGGGGSGGGGSEIVLTQSPATLSLSPGER |
| | | ATLSCSASSSVSYMHWYQQKPGQAPRLLIYDTSKLASGIPARFSGS |
| | | GSGTDFTLTISSLEPEDFAVYYCFQGSVYPFTFGQGTKLEIKRHHH |
| | | HHHHHHH (SEQ ID NO: 312) |
| tAb0038 | HSA:CD19: | EVQLVESGGGLVQPGNSLRLSCAASGFTFSSFGMSWVRQAPGKGL |
| | CD3 | EWVSSISGSGSDTLYADSVKGRFTISRDNAKTTLYLQMNSLRPEDT |
| | | AVYYCTIGGSLSRSSQGTLVTVSSGGGGSGGGSQVQLQESGPGLV |
| | | KPSQTLSLTCTVSGGSISTSGMGVGWIRQHPGKGLEWIGHIWWDD |
| | | DKRYNPALKSRVTISVDTSKNQFSLKLSSVTAADTAVYYCARMEL |
| | | WSYYFDYWGQGTLVTVSSGGGGSGGGGSGGGGSEIVLTQSPATL |
| | | SLSPGERATLSCSASSSVSYMHWYQQKPGQAPRLLIYDTSKLASGI |
| | | PARFSGSGSGTDFTLTISSLEPEDFAVYYCFQGSVYPFTFGQGTKLE |
| | | IKRGGGGSGGGSDIVMTQSPDSLAVSLGERATINCKSSQSLLNART |
| | | GKNYLAWYQQKPGQPPKLLIYWASTRESGVPDRFSGSGSGTDFTL |
| | | TISSLQAEDVAVYYCKQSYSRRTFGGGTKVEIKGGGGSGGGGSGG |
| | | GGSGGGGSQVQLVQSGAEVKKPGASVKVSCKASGFNIKDYYMH |
| | | WVRQAPGQRLEWMGWIDLENANTIYDAKFQGRVTITRDTSASTA |
| | | YMELSSLRSEDTAVYYCARDAYGRYFYDVWGQGTLVTVSSHHH |
| | | HHHHHHH (SEQ ID NO: 313) |
| tAb0042 | CD3:HSA: | EVQLVESGGGLVQPGGSLKLSCAASGFTFNKYAINWVRQAPGKG |
| | CD19 | LEWVARIRSKYNNYATYYADQVKDRFTISRDDSKNTAYLQMNNL |
| | | KTEDTAVYYCVRHANFGNSYISYWAYWGQGTLVTVSSGGGGSG |
| | | GGGSGGGGSQTVVTQEPSLTVSPGGTVTLTCASSTGAVTSGNYPN |
| | | WVQQKPGQAPRGLIGGTKFLVPGTPARFSGSLLGGKAALTLSGVQ |
| | | PEDEAEYYCTLWYSNRWVFGGGTKLTVLGGGGSGGGSEVQLVES |
| | | GGGLVQPGNSLRLSCAASGFTFSSFGMSWVRQAPGKGLEWVSSIS |
| | | GSGSDTLYADSVKGRFTISRDNAKTTLYLQMNSLRPEDTAVYYCT |
| | | IGGSLSRSSQGTLVTVSSGGGGSGGGSQVQLQESGPGLVKPSQTLS |
| | | LTCTVSGGSISTSGMGVGWIRQHPGKGLEWIGHIWWDDDKRYNP |
| | | ALKSRVTISVDTSKNQFSLKLSSVTAADTAVYYCARMELWSYYFD |
| | | YWGQGTLVTVSSGGGGSGGGGSGGGGSEIVLTQSPATLSLSPGER |

The cultures were harvested by centrifugation at 4000 rpm, and the supernatant filtered through a 0.22 mm filter. The multi-specific binding proteins, which carried a 10×His tag at the C-terminus, were purified in two steps. The first step was Nickel affinity chromatography with elution using PBS containing 400 mM imidazole. The second step was size exclusion chromatography with elution in PBS (phosphate buffered saline) pH7.2. Multi-specific binding protein concentrations were determined by UV spectroscopy, and the protein samples were concentrated when necessary. The purity of the proteins was determined by sodium dodecyl sulphate polyacrylamide gel electrophoresis (SDS-PAGE) and high performance liquid chromatography (HPLC). Specifically, HPLC was performed on an Agilent 1100 series instrument using MabPac size exclusion column run in PBS at 0.2 mL/min. The fractions with an elution time of about 225-240 minutes were collected for further characterization.

As noted above, the constructs produced contained an anti-CD19 scFv having the amino acid sequence set forth in SEQ ID NO: 9 or 18. The binding affinity of the two CD19 binding domains to CD19 were measured by SPR using a monomeric CD19 extracellular domain and a dimeric CD19 extracellular domain fused with human IgG1 Fc. Binding kinetic parameters were measured using a ForteBio instrument generally as previously described (see, Estep et al. (2013) MAbs, 5(2): 270-78). When measured with the monomeric CD19 protein, the K_{D} value of the CD19 binding domain having the sequence of SEQ ID NO: 9 was 7 nM, and the K_{D} value of the CD19 binding domain having the sequence of SEQ ID NO: 18 was 11 nM. When measured with the dimeric CD19 protein, the K_{D} value of the CD19 binding domain having the sequence of SEQ ID NO: 9 was 5 nM, and the K_{D} value of the CD19 binding domain having the sequence of SEQ ID NO: 18 was 15 nM.

### Example 3. Multi-Specific Binding Proteins Induce T Cell Cytotoxicity Against CD19⁺ Target Cells

This example describes the cytotoxic activity of multi-specific binding proteins.

The T cell redirection activity of multi-specific binding proteins and BiTE proteins were evaluated using the KILR Raji Cell Model. Briefly, pan T cells were isolated from primary human PBMCs from a single healthy donor by negative selection using a commercial kit (e.g. Easy Sep Human T Cell Enrichment Kit, StemCell Technologies). T cells were maintained in RPMI 1640 medium supplemented with 10% serum and 300 IU/mL IL-2 to expand T cells. The harvested T cells were washed twice to remove any serum.

KILR Raji cells, which expressed CD19 on the surface, were used as target cells. To opsonize the target cells, each multi-specific binding protein or BiTE (see Table 6) was incubated with the target cells for 30 minutes at 37° C. in RPMI 1640 medium supplemented with 5% heat inactivated low IgG fetal bovine serum and penicillin-streptomycin-glutamine. The proteins were added in serial dilution at 10 different doses, with each dose run in duplicate. Human serum albumin was added to the medium of certain samples at a final concentration of 15 mg/mL. Selective proteins were also evaluated with KILR SKOV3 cells, which were CD19-negative, as negative controls.

After opsonization, the target cells were incubated with the pan T cells at an effector-to-target (E:T) ratio of 10:1 for 6 hours at 37° C. Killing of the KILR Raji cells resulted in release of a labeled housekeeping protein from these cells into the medium, which was quantified by addition of a KILR detection reagent (DiscoverX). The luminescence signals from all wells were read on an Envision plate reader. Spontaneous release and total lysis controls were included on each plate to allow calculation of percent killing.

Percent killing was calculated from the luminescence signal values using the following formula:

% killing=(value from test protein sample−mean value from spontaneous release control)/(mean value from total lysis control−mean value from spontaneous release control)×100.

The EC50 values were calculated from the percent killing by fitting with a dose-response curve using the GraphPad Prism software.

Table 7 lists the EC50 values of T cell-redirected killing in the absence and presence of human serum albumin for exemplary multi-specific binding proteins and comparator anti-CD19 BiTE protein. No substantial killing was observed with the CD19-negative KILR SKOV3 cells.

TABLE 7
Cytotoxic Activity of Multi-specific Binding Proteins

| | | EC50 (pg/mL) | | Fold |
|---|---|---|---|---|
| Construct | Format | −HSA | +HSA | change |
| tAb0027 | CD19:CD3:HSA | 2.33 | 67.9 | 29.1 |
| tAb0029 | CD3:CD19:HSA | 5.48 | 188.1 | 34.3 |
| tAb0030 | CD3:HSA:CD19 | 3.17 | 141.7 | 44.7 |
| tAb0031 | HSA:CD3:CD19 | 6.55 | 189.3 | 28.9 |
| tAb0032 | HSA:CD19:CD3 | 5.71 | 88.4 | 15.5 |
| tAb0033 | CD19:CD3:HSA | 20.4 | 1294 | 63.4 |
| tAb0034 | CD19:HSA:CD3 | 59.3 | 2376 | 40.1 |
| tAb0035 | CD3:CD19:HSA | 51.4 | 2184 | 42.5 |
| tAb0036 | CD3:HSA:CD19 | 103.8 | 4195 | 40.4 |
| tAb0037 | HSA:CD3:CD19 | 175.1 | 2151 | 12.3 |
| tAb0038 | HSA:CD19:CD3 | 52.6 | 288 | 5.5 |
| tAb0042 | CD3:HSA:CD19 | 69.9 | 13290 | 190.1 |
| blinatumomab | CD19:CD3 | 2854 | 10750 | 3.8 |

As shown in Table 7, the multi-specific binding proteins containing the anti-CD19 scFv having the amino acid sequence of SEQ ID NO: 9 showed stronger cytotoxic activity than those containing the anti-CD19 scFv having the amino acid sequence of SEQ ID NO: 18, regardless of the construct format, CD3 binding domain, HSA binding domain, and the presence or absence of HSA in the assay medium. From this data, it is contemplated that constructs containing this anti-CD19 scFv with the higher binding affinity to CD19 will demonstrate stronger therapeutic activity than constructs containing the other anti-CD19 scFv with the lower binding affinity.

Furthermore, all the multi-specific binding proteins tested showed lower EC₅₀ value (namely, stronger ability to induce cytotoxicity) in the absence of HSA than in the presence of HSA. Without wishing to be bound by theory, it appears that the presence of HSA causes a change in the protein complex, which was specific to the multi-specific binding proteins containing an HSA binding domain, rather than a nonspecific effect as observed with blinatumomab. The ratio of the EC₅₀ value in the presence of HSA to the EC₅₀ value in the absence of HSA, also called “fold change” herein, was used to assess the effect of HSA on the potential therapeutic activity of the multi-specific binding protein. As shown in Table 7, the construct formats with the HSA binding domain N-terminal to both the CD19 binding domain and the CD3 binding domain (namely, tAb0031, tAb0032, tAb0037, and tAb0038) showed lower fold changes than the other construct formats, regardless of which CD19 binding domain was used in the construct.

Furthermore, among the constructs having the same CD19 binding domain, CD3 binding domain, and HSA binding domain, the constructs in the CD19:CD3:HSA format (i.e., the CD19 binding domain positioned N-terminal to the CD3 binding domain, and the CD3 binding domain positioned N-terminal to the HSA binding domain), namely, tAb0027 and tAb0033, showed the lowest or second lowest EC₅₀ values both in the absence and in the presence of HSA.

### Example 4. Cytotoxicity of Multi-Specific Binding Proteins Against CD19⁺ Target Cells

This example provides alternative methods for determining the cytotoxic activity of a multi-specific binding protein.

The multi-specific binding proteins disclosed herein can be evaluated in in vitro assays on their mediation of T cell dependent cytotoxicity to B cell antigen positive target cells. For example, the CD19-binding multi-specific binding protein disclosed herein is evaluated in in vitro assays on its mediation of T cell dependent cytotoxicity to CD19⁺ target cells.

Fluorescence labeled CD19⁺ MEC-1 cells (a CD19+ human chronic B cell leukemia cell line) are incubated with isolated PBMC of random donors or CB15 T-cells (standardized T-cell line) as effector cells in the presence of the CD19-binding multi-specific binding protein. After incubation for 4 hours at 37° C. in a humidified incubator, the release of the fluorescent dye from the target cells into the supernatant is determined in a spectrofluorimeter. Target cells incubated without the CD19-binding multi-specific binding protein and target cells totally lysed by the addition of saponin at the end of the incubation serve as negative and positive controls, respectively. Based on the measured remaining living target cells, the percentage of specific cell lysis can be calculated according to the following formula: [1−(number of living targets₍ₛₐₘₚₗₑ₎/number of living targets₍ₛₚₒₙₜₐₙₑₒᵤₛ₎]×100%. Sigmoidal dose response curves and EC₅₀ values are calculated by non-linear regression/4-parameter logistic fit using the GraphPad Software. The lysis values obtained for a given multi-specific binding protein concentration are used to calculate sigmoidal dose-response curves by 4 parameter logistic fit analysis using the Prism software. It is expected that the target cell lysis rate induced by CD19-binding multi-specific binding protein is higher than the target cell lysis rate induced by similar constructs lacking either a CD19-binding domain or a CD3-binding domain.

Alternatively, a human T-cell dependent cellular cytotoxicity (TDCC) assay is used to measure the ability of the multi-specific binding protein to direct T cells to kill tumor cells (Nazarian et al. 2015, J Biomol. Screen, 20:519-27). In this assay, T cells and target cancer cell line cells are mixed together at a 10:1 ratio in a 384 wells plate, and varying amounts of the multi-specific binding proteins are added. After 48 hours, the T cells are washed away leaving attached to the plate target cells that were not killed by the T cells. To quantitate the remaining viable cells, CellTiter-Glo® Luminescent Cell Viability Assay (Promega) is used. It is contemplated that the killing rate of B-cell antigen expressing cancer cell induced by CD19-binding multi-specific binding protein will be higher than that induced by similar constructs lacking either a CD19-binding domain or a CD3-binding domain and/or other negative control molecules.

### Example 5. Pharmacokinetics of Multi-Specific Binding Proteins with HSA Binding Domain

This example is designed to determine the pharmacokinetics of multi-specific binding proteins.

Multi-specific binding proteins containing a domain that binds CD19, a domain that binds CD3, and a domain that binds serum albumin are tested in the cynomolgus monkey in the context of pharmacokinetic (PK) studies to evaluate the serum elimination time of the multi-specific binding protein.

The multi-specific binding proteins are administered as intravenous bolus or intravenous infusion. The multi-specific binding proteins are administered in a dose-linear, pharmacokinetic relevant range of 0.5 μg/kg to 3 μg/kg, 6 μg/kg, 12 μg/kg, and 15 μg/kg, respectively. For purposes of comparability, the serum concentrations of the multi-specific binding proteins are does-normalized and molecular weight-normalized (described in nmol).

For each multi-specific binding protein, a group of at least two to three animals are used. Blood samples are collected and serum is prepared for determination of serum concentrations of the multi-specific binding proteins. Serum multi-specific binding protein levels are measured using an immunoassay. The assay is performed by capturing the multi-specific binding protein via its CD19-binding domain, while an antibody directed against the CD3-binding domain of the multi-specific binding protein is used for detection. The serum concentration-time profiles are used to determine PK parameters using known analytical methods such as those described in Ritschel W A and Kearns G L, 1999, IN: Handbook Of Basic Pharmacokinetics Including Clinical Applications, 5th edition, American Pharmaceutical Assoc., Washington, D.C. and softwares such as WinNonlin software (WinNonlin® Professional V. 3.1 WinNonlin™ Copyright 1998-1999. Pharsight Corporation. Mountain View, Calif.).

Alternatively, the serum half-life of the various multi-specific binding proteins containing the serum albumin binding domain is compared to that of control constructs capable of binding CD19 and CD3 but lacking a serum albumin binding domain by including in the experiment another cynomolgus monkey group that receives the control constructs. Additional domains can be included such that the control constructs are similar in size to the multi-specific binding proteins.

It is expected that CD19-binding multi-specific binding protein will have significantly longer serum half-life compared to similar constructs capable of binding CD19 and CD3 but lacking a serum albumin binding domain and/or other negative control molecules.

### Example 6. Determination of Antigen Affinity by Flow Cytometry

This example is designed to determine the affinity of a multi-specific binding protein to an antigen.

Various multi-specific binding proteins disclosed herein are tested for their binding affinities to human CD3⁺ cells and the corresponding B cell surface antigen positive cells, such as human CD19⁺ cells. The multi-specific binding proteins are also tested for their binding affinities to cynomolgus CD3⁺ cells and the corresponding B cell surface antigen positive cells, such as cynomolgus CD19⁺ cells.

CD3⁺ and CD19⁺ cells are incubated with 100 μL of serial dilutions of the multi-specific binding protein. After washing three times with FACS buffer the cells are incubated with 0.1 mL of 10 pg/mL mouse monoclonal anti-idiotype antibody in the same buffer for 45 mins on ice. After a second washing cycle, the cells are incubated with 0.1 mL of 15 pg/mL FITC-conjugated goat anti-mouse IgG antibodies under the same conditions as before. As a control, cells are incubated with the anti-His IgG followed by the FITC-conjugated goat anti-mouse IgG antibodies without the multi-specific binding protein. The cells are then washed again and resuspended in 0.2 mL of FACS buffer containing 2 pg/mL propidium iodide (PI) in order to exclude dead cells. The fluorescence of 1×10⁴ living cells is measured using a commercially available flow cytometer and software. Mean fluorescence intensities of the cell samples are calculated using software such as CXP software (Beckman-Coulter, Krefeld, Germany) or Incyte software (Merck Millipore, Schwalbach, Germany). K_{D} values for one-site binding can be calculated using normalized fluorescence intensity values with known computational equations such as those supplied in the GraphPad Prism software (GraphPad Software, La Jolla Calif. USA). CD3 binding affinity and cross-reactivity are evaluated in titration and flow cytometric experiments on CD3⁺ Jurkat cells and the cynomolgus CD3⁺ HSC-F cell line. CD19 binding and cross-reactivity are assessed on the human CD19⁺ tumor cell lines. The K_{D} ratio of cross-reactivity can be calculated using the K_{D} values determined on the CHO cell lines expressing either recombinant human or recombinant cynomolgus antigens.

### Example 7. Cytokine Production Induced by Multi-Specific Binding Proteins

This example is designed to determine the ability of a multi-specific binding protein to induce cytokine production from immune cells.

AlphaLISA assays (Perkin Elmer) for TNFα and Interferon γ are used to obtain evidence that T cells are activated by the multi-specific binding proteins of current invention, such as CD19-binding multi-specific binding protein, in the presence of target cells, such as CD19⁺ B cells. For this assay, primary human T cells and human tumor cells expressing B cell surface antigen are incubated in the presence of the CD19-binding multi-specific binding protein as described under cytotoxicity assays. After 48 hours of incubation, 2 microliter aliquots of the assay supernatants are analyzed according to the manufacturer's instructions. It is contemplated that the TNFα or Interferon γ level induced by CD19-binding multi-specific binding protein is higher than that induced by similar constructs lacking either a CD19-binding domain or a CD3-binding domain and/or other negative control molecules.

### INCORPORATION BY REFERENCE

All publications and patents cited throughout the text of this specification (including all patents, patent applications, scientific publications, manufacturer's specifications, instructions, etc.), whether supra or infra, are hereby incorporated by reference in their entirety for all purposes. To the extent the material incorporated by reference contradicts or is inconsistent with this specification, the specification will supersede any such material.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. An antigen-binding site that binds human serum albumin, comprising a VH comprising complementarity determining regions HCDR1, HCDR2, and HCDR3, wherein the HCDR1, HCDR2, and HCDR3 comprise the amino acid sequences of SEQ ID NOs: 184, 409, and 411, respectively, but not SEQ ID NOs: 129, 133, and 135, respectively.

2. The antigen-binding site of claim 1, wherein the HCDR1, HCDR2, and HCDR3 comprise the amino acid sequences of SEQ ID NOs: 184, 185, and 187, respectively, but not SEQ ID NOs: 129, 133, and 135, respectively.

3. The antigen-binding site of claim 1, wherein the HCDR1, HCDR2, and HCDR3 comprise the amino acid sequences of SEQ ID NOs: 189, 190, and 192, respectively, but not SEQ ID NOs: 129, 133, and 135, respectively.

4. The antigen-binding site of any one of claims 1-3, wherein the HCDR1, HCDR2, and HCDR3 comprise the amino acid sequences of SEQ ID NOs: 189, 193, and 195, respectively, but not SEQ ID NOs: 129, 133, and 135, respectively.

5. The antigen-binding site of claim 4, wherein the HCDR1, HCDR2, and HCDR3 comprise the amino acid sequences of SEQ ID NOs: 123, 124, and 126, respectively.

6. The antigen-binding site of any one of claims 1-5, wherein the VH comprises an amino acid sequence at least 85%, at least 90%, at least 95%, or at least 99% identical to SEQ ID NO: 121.

7. The antigen-binding site of any one of claims 1-6, wherein the VH comprises the amino acid sequence of SEQ ID NO: 121.

8. The antigen-binding site of any one of claims 1-7, wherein the antigen-binding site binds human serum albumin with a K_{D} lower than or equal to 10 nM.

9. The antigen-binding site of any one of claims 1-8, wherein the antigen-binding site binds protein A with a K_{D} lower than or equal to 2 nM.

10. The antigen-binding site of any one of claims 1-9, wherein the antigen-binding site has a melting temperature greater than or equal to 60° C.

11. A multi-specific binding protein comprising:
(a) a first antigen-binding site that binds a first target protein expressed on a target cell;
(b) a second antigen-binding site that binds a second target protein expressed on an immune effector cell; and
(c) a third antigen-binding site that binds human serum albumin, wherein the third antigen-binding site is an antigen-binding site of any one of claims 1-10.

12. The multi-specific binding protein of claim 11, wherein the first antigen-binding site binds human CD19.

13. The multi-specific binding protein of claim 11 or 12, wherein the second antigen-binding site binds human CD3.

14. The multi-specific binding protein of any one of claims 11-13, wherein the multi-specific binding protein comprises a single polypeptide chain.

15. The multi-specific binding protein of claim 14, wherein the third antigen-binding site is not positioned between the first antigen-binding site and the second antigen-binding site in the polypeptide chain.

16. The multi-specific binding protein of claim 15, wherein the third antigen-binding site is positioned N-terminal to both the first antigen-binding site and the second antigen-binding site in the polypeptide chain.

17. The multi-specific binding protein of claim 16, wherein the third antigen-binding site is positioned N-terminal to the first antigen-binding site, and the first antigen-binding site is positioned N-terminal to the second antigen-binding site in the polypeptide chain.

18. The multi-specific binding protein of claim 16, wherein the third antigen-binding site is positioned N-terminal to the second antigen-binding site, and the second antigen-binding site is positioned N-terminal to the first antigen-binding site in the polypeptide chain.

19. The multi-specific binding protein of claim 15, wherein the third antigen-binding site is positioned C-terminal to both the first antigen-binding site and the second antigen-binding site in the polypeptide chain.

20. The multi-specific binding protein of claim 19, wherein the first antigen-binding site is positioned N-terminal to the second antigen-binding site, and the second antigen-binding site is positioned N-terminal to the third antigen-binding site in the polypeptide chain.

21. The multi-specific binding protein of claim 19, wherein the second antigen-binding site is positioned N-terminal to the first antigen-binding site, and the first antigen-binding site is positioned N-terminal of the third antigen-binding site in the polypeptide chain.

22. The multi-specific binding protein of claim 14, wherein the first antigen-binding site is positioned N-terminal to the third antigen-binding site, and the third antigen-binding site is positioned N-terminal to the second antigen-binding site in the polypeptide chain.

23. The multi-specific binding protein of claim 14, wherein the second antigen-binding site is positioned N-terminal to the third antigen-binding site, and the third antigen-binding site is positioned N-terminal binding protein the first antigen-binding site in the polypeptide chain.

24. The multi-specific binding protein of any one of claims 11-23, wherein the first antigen-binding site comprises a single-chain variable fragment (scFv).

25. The multi-specific binding protein of any one of claims 11-24, wherein the third antigen-binding site comprises a single domain antibody (sdAb).

26. The multi-specific binding protein of any one of claims 11-25, wherein the second antigen-binding site comprises an scFv.

27. The multi-specific binding protein of any one of claims 13-26, wherein the second antigen-binding site binds human CD3ε.

28. The multi-specific binding protein of claim 27, wherein the second antigen-binding site binds human CD3ε with a K_{D} in the range of 1-100 nM.

29. The multi-specific binding protein of any one of claims 13-28, wherein the second antigen-binding site comprises a VH comprising complementarity determining regions HCDR1, HCDR2, and HCDR3, and a VL comprising complementarity determining regions LCDR1, LCDR2, and LCDR3, wherein the HCDR1, HCDR2, HCDR3, LCDR1, LCDR2, and LCDR3 comprise the amino acid sequences set forth in SEQ ID NOs: 415, 416, 418, 419, 420, and 421, respectively.

30. The multi-specific binding protein of claim 29, wherein the VH comprises an amino acid sequence at least 85%, at least 90%, at least 95%, at least 99%, or 100% identical to SEQ ID NO: 412, and the VL comprises an amino acid sequence at least 85%, at least 90%, at least 95%, at least 99%, or 100% identical to SEQ ID NO: 413.

31. The multi-specific binding protein of claim 29 or 30, wherein the antigen-binding site comprises the amino acid sequence of SEQ ID NO: 422 or 423.

32. The multi-specific binding protein of any one of claims 13-28, wherein the second antigen-binding site comprises a VH comprising complementarity determining regions HCDR1, HCDR2, and HCDR3, and a VL comprising complementarity determining regions LCDR1, LCDR2, and LCDR3, wherein the HCDR1, HCDR2, HCDR3, LCDR1, LCDR2, and LCDR3 comprise the amino acid sequences set forth in SEQ ID NOs: 415, 416, 426, 419, 420, and 421, respectively.

33. The multi-specific binding protein of claim 32, wherein the VH comprises an amino acid sequence at least 85%, at least 90%, at least 95%, at least 99%, or 100% identical to SEQ ID NO: 424, and the VL comprises an amino acid sequence at least 85%, at least 90%, at least 95%, at least 99%, or 100% identical to SEQ ID NO: 413.

34. The multi-specific binding protein of claim 32 or 33, wherein the antigen-binding site comprises the amino acid sequence of SEQ ID NO: 427 or 428.

35. The multi-specific binding protein of any one of claims 13-28, wherein the second antigen-binding site comprises a VH comprising complementarity determining regions HCDR1, HCDR2, and HCDR3, and a VL comprising complementarity determining regions LCDR1, LCDR2, and LCDR3, wherein the HCDR1, HCDR2, HCDR3, LCDR1, LCDR2, and LCDR3 comprise the amino acid sequences set forth in SEQ ID NOs: 415, 431, 418, 419, 420, and 432, respectively.

36. The multi-specific binding protein of claim 35, wherein the VH comprises an amino acid sequence at least 85%, at least 90%, at least 95%, at least 99%, or 100% identical to SEQ ID NO: 429, and the VL comprises an amino acid sequence at least 85%, at least 90%, at least 95%, at least 99%, or 100% identical to SEQ ID NO: 430.

37. The multi-specific binding protein of claim 35 or 36, wherein the antigen-binding site comprises the amino acid sequence of SEQ ID NO: 433 or 434.

38. The multi-specific binding protein of any one of claims 11-37, wherein at least two adjacent antigen-binding sites are connected by a peptide linker.

39. The multi-specific binding protein of claim 38, wherein each of the adjacent antigen-binding sites are connected by a peptide linker.

40. The multi-specific binding protein of claim 38 or 39, wherein the peptide linker comprises the amino acid sequence of SEQ ID NO: 298, 299, or 302.

41. The multi-specific binding protein of claim 38 or 39, wherein the peptide linker consists of the amino acid sequence of SEQ ID NO: 298, 299, or 302.

42. The multi-specific binding protein of any one of claims 11-41, wherein the multi-specific binding protein does not comprise an antibody Fc region.

43. The multi-specific binding protein of any one of claims 11-42, wherein the molecular weight of the multi-specific binding protein is at least 65 kD.

44. The multi-specific binding protein of any one of the preceding claims, wherein the serum half-life of the multi-specific binding protein is at least 24, 36, 48, or 60 hours.

45. An antibody comprising the antigen-binding site of any one of claims 1-10.

46. A pharmaceutical composition comprising:
(a) the multi-specific binding protein of any one of claims 11-44 or the antibody of claim 45; and
(b) a pharmaceutically acceptable carrier.

47. An isolated polynucleotide encoding the multi-specific binding protein of any one of claims 11-44 or the antibody of claim 45.

48. A vector comprising the polynucleotide of claim 47.

49. A recombinant host cell comprising the polynucleotide of claim 47 or the vector of claim 48.

50. A method of producing a multi-specific binding protein or an antibody, the method comprising culturing the host cell of claim 49 under suitable conditions that allow expression of the multi-specific binding protein or the antibody.

51. The method of claim 50, further comprising isolating the multi-specific binding protein or the antibody.

52. The method of claim 51, further comprising formulating the isolated multi-specific binding protein or antibody with a pharmaceutically acceptable carrier.

53. A method of stimulating an immune response against a target cell, the method comprising exposing the cell and a T lymphocyte to the multi-specific binding protein of any one of claims 11-44, the antibody of claim 45, or the pharmaceutical composition of claim 46.

54. A method of treating a hematologic cancer in a subject in need thereof, the method comprising administering to the subject an effective amount of the multi-specific binding protein of any one of claims 11-44, the antibody of claim 45, or the pharmaceutical composition of claim 46.

55. The method of claim 54, wherein the hematologic cancer is a B-cell hematologic malignancy.

56. A complex comprising a T cell expressing CD3, a B cell expressing CD19, and the multi-specific binding protein of any one of claims 13-44, wherein the multi-specific binding protein simultaneously bind both the T cell and the B cell.

57. The complex of claim 56, further comprising serum albumin.


SEQUENCE LISTING (719 sequences)

SEQ ID NO: 1-3 - skipped (3 entries)

SEQ ID NO: 4 - protein, 5 aa, Artificial Sequence
  Description of Artificial Sequence: Synthetic peptide
DYIMH

SEQ ID NO: 5-9 - skipped (5 entries)

SEQ ID NO: 10 - protein, 9 aa, Artificial Sequence
  Description of Artificial Sequence: Synthetic peptide
LQLLEDPYT

SEQ ID NO: 11-12 - skipped (2 entries)

SEQ ID NO: 13 - protein, 112 aa, Artificial Sequence
  Description of Artificial Sequence: Synthetic polypeptide
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 14-15 - skipped (2 entries)

SEQ ID NO: 16 - protein, 14 aa, Artificial Sequence
  Description of Artificial Sequence: Synthetic peptide
ARGTYYYGPQLFDY

SEQ ID NO: 17 - protein, 12 aa, Artificial Sequence
  Description of Artificial Sequence: Synthetic peptide
GTYYYGPQLFDY

SEQ ID NO: 18 - protein, 16 aa, Artificial Sequence
  Description of Artificial Sequence: Synthetic peptide
KSSQSLETSTGTTYLN

SEQ ID NO: 19 - protein, 7 aa, Artificial Sequence
  Description of Artificial Sequence: Synthetic peptide
RVSKRFS

SEQ ID NO: 20 - protein, 121 aa, Artificial Sequence
  Description of Artificial Sequence: Synthetic polypeptide
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSS

SEQ ID NO: 21 - skipped

SEQ ID NO: 22 - protein, 9 aa, Artificial Sequence
  Description of Artificial Sequence: Synthetic peptide
YTFTDYIMH

SEQ ID NO: 23 - protein, 17 aa, Artificial Sequence
  Description of Artificial Sequence: Synthetic peptide
YINPYNDGSKYTEKFQG

SEQ ID NO: 24-28 - skipped (5 entries)

SEQ ID NO: 29 - protein, 5 aa, Artificial Sequence
  Description of Artificial Sequence: Synthetic peptide
SYWMN

SEQ ID NO: 30-66 - skipped (37 entries)

SEQ ID NO: 67 - protein, 12 aa, Artificial Sequence
  Description of Artificial Sequence: Synthetic peptide
ARMELWSYYFDY

SEQ ID NO: 68 - protein, 10 aa, Artificial Sequence
  Description of Artificial Sequence: Synthetic peptide
MELWSYYFDY

SEQ ID NO: 69 - skipped

SEQ ID NO: 70 - protein, 7 aa, Artificial Sequence
  Description of Artificial Sequence: Synthetic peptide
DTSKLAS

SEQ ID NO: 71 - protein, 9 aa, Artificial Sequence
  Description of Artificial Sequence: Synthetic peptide
FQGSVYPFT

SEQ ID NO: 72-73 - skipped (2 entries)

SEQ ID NO: 74 - protein, 106 aa, Artificial Sequence
  Description of Artificial Sequence: Synthetic polypeptide
EIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIK

SEQ ID NO: 75-79 - skipped (5 entries)

SEQ ID NO: 80 - protein, 10 aa, Artificial Sequence
  Description of Artificial Sequence: Synthetic peptide
SASSSVSYMH

SEQ ID NO: 81-86 - skipped (6 entries)

SEQ ID NO: 87 - protein, 16 aa, Artificial Sequence
  Description of Artificial Sequence: Synthetic peptide
HIWWDDDKRYNPALKS

SEQ ID NO: 88-89 - skipped (2 entries)

SEQ ID NO: 90 - protein, 120 aa, Artificial Sequence
  Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 91 - skipped

SEQ ID NO: 92 - protein, 11 aa, Artificial Sequence
  Description of Artificial Sequence: Synthetic peptide
GSISTSGMGVG

SEQ ID NO: 93 - protein, 7 aa, Artificial Sequence
  Description of Artificial Sequence: Synthetic peptide
TSGMGVG

SEQ ID NO: 94-120 - skipped (27 entries)

SEQ ID NO: 121 - protein, 115 aa, Artificial Sequence
  Description of Artificial Sequence: Synthetic polypeptide
KVQLVESGGGLVQPGGSLRLSCAASGFTFSSFGMTWVRQAPGKGLEWVSSISGSGSDTLYADSVRGRFTISRDNSKNTLYLQMNSLRAEDTAVYYCTIGGSLSPSSQGTLVTVSS

SEQ ID NO: 122 - protein, 9 aa, Artificial Sequence
  Description of Artificial Sequence: Synthetic peptide
FTFSSFGMT

SEQ ID NO: 123 - protein, 5 aa, Artificial Sequence
  Description of Artificial Sequence: Synthetic peptide
SFGMT

SEQ ID NO: 124 - protein, 17 aa, Artificial Sequence
  Description of Artificial Sequence: Synthetic peptide
SISGSGSDTLYADSVRG

SEQ ID NO: 125 - protein, 8 aa, Artificial Sequence
  Description of Artificial Sequence: Synthetic peptide
TIGGSLSP

SEQ ID NO: 126 - protein, 6 aa, Artificial Sequence
  Description of Artificial Sequence: Synthetic peptide
GGSLSP

SEQ ID NO: 127 - protein, 115 aa, Artificial Sequence
  Description of Artificial Sequence: Synthetic polypeptide
KVQLLESGGGLVQPGGSLRLSCAASGFTFSSFGMSWVRQAPGKGLEWVSSISGSGSDTLYADSVRGRFTISRDNSKNTLYLQMNSLRPEDTAVYYCTIGGSLSPSSQGTLVTVSS

SEQ ID NO: 128 - protein, 9 aa, Artificial Sequence
  Description of Artificial Sequence: Synthetic peptide
FTFSSFGMS

SEQ ID NO: 129 - protein, 5 aa, Artificial Sequence
  Description of Artificial Sequence: Synthetic peptide
SFGMS

SEQ ID NO: 130 - protein, 115 aa, Artificial Sequence
  Description of Artificial Sequence: Synthetic polypeptide
KVQLVESGGGLVQPGGSLRLSCAASGFTFSSFGMTWVRQAPGKGLEWVSSISGSGSDTLYADSVRGRFTISRDNSKNTLYLQMNSLRPEDTAVYYCTIGGSLSPSSQGTLVTVSS

SEQ ID NO: 131 - protein, 115 aa, Artificial Sequence
  Description of Artificial Sequence: Synthetic polypeptide
KVQLLESGGGLVQPGGSLRLSCAASGFTFHSFGMSWVRQAPGKGLEWVSSISGSGSDTLYADSVKGRFTISRDNSKNTLYLQMNSLRPEDTAVYYCTIGGSLSRSSQGTLVTVSS

SEQ ID NO: 132 - protein, 9 aa, Artificial Sequence
  Description of Artificial Sequence: Synthetic peptide
FTFHSFGMS

SEQ ID NO: 133 - protein, 17 aa, Artificial Sequence
  Description of Artificial Sequence: Synthetic peptide
SISGSGSDTLYADSVKG

SEQ ID NO: 134 - protein, 8 aa, Artificial Sequence
  Description of Artificial Sequence: Synthetic peptide
TIGGSLSR

SEQ ID NO: 135 - protein, 6 aa, Artificial Sequence
  Description of Artificial Sequence: Synthetic peptide
GGSLSR

SEQ ID NO: 136 - protein, 115 aa, Artificial Sequence
  Description of Artificial Sequence: Synthetic polypeptide
EVQLLESGGGLVQPGGSLRLSCAASGFVFSSFGMSWVRQAPGKGLEWVSSISGSGSDTLYADSVKGRFTISRDNSKNTLYLQMNSLRPEDTAVYYCTIGGSLSRSSQGTLVTVSS

SEQ ID NO: 137 - protein, 9 aa, Artificial Sequence
  Description of Artificial Sequence: Synthetic peptide
FVFSSFGMS

SEQ ID NO: 138 - protein, 115 aa, Artificial Sequence
  Description of Artificial Sequence: Synthetic polypeptide
KVQLLESGGGLVQPGGSLRLSCAASGFTFSSFGMSWVRQAPGKGLEWVSSISGSGSDTLYADSVRGRFTISRDNSKNTLYLQMDSLRAEDTAVYYCTIGGSRSISSQGTLVTVSS

SEQ ID NO: 139 - protein, 8 aa, Artificial Sequence
  Description of Artificial Sequence: Synthetic peptide
TIGGSRSI

SEQ ID NO: 140 - protein, 6 aa, Artificial Sequence
  Description of Artificial Sequence: Synthetic peptide
GGSRSI

SEQ ID NO: 141 - protein, 115 aa, Artificial Sequence
  Description of Artificial Sequence: Synthetic polypeptide
KVQLLESGGGLVQPGGSLRLSCAASGFTFSSFGMSWVRQAPGKGLEWVSSISGSGSDTLYADSVRGRFTISRDNSKNTLYLQMNSLRAEDTAVYYCSIGGSLIRSSQGTLVTVSS

SEQ ID NO: 142 - protein, 8 aa, Artificial Sequence
  Description of Artificial Sequence: Synthetic peptide
SIGGSLIR

SEQ ID NO: 143 - protein, 6 aa, Artificial Sequence
  Description of Artificial Sequence: Synthetic peptide
GGSLIR

SEQ ID NO: 144 - protein, 115 aa, Artificial Sequence
  Description of Artificial Sequence: Synthetic polypeptide
KVQLVESGGGLVQPGGSLRLSCAASGFTFGSFGMSWVRQAPGKGLEWVSSISGSGADTLYADSVKGRFTISRDNSKNTLYLQMNSLRAEDTAVYYCTIGGSLRASSQGTLVTVSS

SEQ ID NO: 145 - protein, 9 aa, Artificial Sequence
  Description of Artificial Sequence: Synthetic peptide
FTFGSFGMS

SEQ ID NO: 146 - protein, 17 aa, Artificial Sequence
  Description of Artificial Sequence: Synthetic peptide
SISGSGADTLYADSVKG

SEQ ID NO: 147 - protein, 8 aa, Artificial Sequence
  Description of Artificial Sequence: Synthetic peptide
TIGGSLRA

SEQ ID NO: 148 - protein, 6 aa, Artificial Sequence
  Description of Artificial Sequence: Synthetic peptide
GGSLRA

SEQ ID NO: 149 - protein, 115 aa, Artificial Sequence
  Description of Artificial Sequence: Synthetic polypeptide
KVQLVESGGGLVQPGNSLRLSCAASGFTFGSFGMSWVRQAPGKGPEWVSSISGSGSDTLYADSVKGRFTISRDNSKNTLYLQMNSLRPEDTAVYYCTIGGSLSRSSQGTLVTVSS

SEQ ID NO: 150 - protein, 115 aa, Artificial Sequence
  Description of Artificial Sequence: Synthetic polypeptide
KVQLVESGGGLVQPGNSLRLSCAASGFTFSSFGMSWVRQAPGKGLEWVSSISGSGGDTLYADSAKGRFTISRDNSKNTLYLQMNSLRPEDTAVYYCTIGGSLSRSSQGTLVTVSS

SEQ ID NO: 151 - protein, 17 aa, Artificial Sequence
  Description of Artificial Sequence: Synthetic peptide
SISGSGGDTLYADSAKG

SEQ ID NO: 152 - protein, 115 aa, Artificial Sequence
  Description of Artificial Sequence: Synthetic polypeptide
KVQLVESGGGLVQPGGSLRLSCAASGFTFSSFGMSWVRQAPGKGLEWVSSISGSGSDTLYADSVEGRFTISRDNSKNTLYLQMNSLRAEDTAVYYCTIGGSLKQSSQGTLVTVSS

SEQ ID NO: 153 - protein, 17 aa, Artificial Sequence
  Description of Artificial Sequence: Synthetic peptide
SISGSGSDTLYADSVEG

SEQ ID NO: 154 - protein, 8 aa, Artificial Sequence
  Description of Artificial Sequence: Synthetic peptide
TIGGSLKQ

SEQ ID NO: 155 - protein, 6 aa, Artificial Sequence
  Description of Artificial Sequence: Synthetic peptide
GGSLKQ

SEQ ID NO: 156 - protein, 115 aa, Artificial Sequence
  Description of Artificial Sequence: Synthetic polypeptide
KVQLLESGGGLVQPGGSLRLSCAASGFTFPSFGMSWVRQAPGKGLEWVSSISGSGSDTLYADSVKGRFTISRDNAKTTLYLQMNSLRPEDTAVYYCTIGGSVSKSSRGTLVTVSS

SEQ ID NO: 157 - protein, 9 aa, Artificial Sequence
  Description of Artificial Sequence: Synthetic peptide
FTFPSFGMS

SEQ ID NO: 158 - protein, 8 aa, Artificial Sequence
  Description of Artificial Sequence: Synthetic peptide
TIGGSVSK

SEQ ID NO: 159 - protein, 6 aa, Artificial Sequence
  Description of Artificial Sequence: Synthetic peptide
GGSVSK

SEQ ID NO: 160 - protein, 115 aa, Artificial Sequence
  Description of Artificial Sequence: Synthetic polypeptide
GVQLLESGGGLVQPGGSLRLSCAASGFTFSSFGMSWVRQAPGKGLEWVSSISGTGSDTLYADSVKGRFTISRDNSKNTLYLQMNSLRPEDTAVYYCTIGGSLRYSSQGTLVTVSS

SEQ ID NO: 161 - protein, 17 aa, Artificial Sequence
  Description of Artificial Sequence: Synthetic peptide
SISGTGSDTLYADSVKG

SEQ ID NO: 162 - protein, 8 aa, Artificial Sequence
  Description of Artificial Sequence: Synthetic peptide
TIGGSLRY

SEQ ID NO: 163 - protein, 6 aa, Artificial Sequence
  Description of Artificial Sequence: Synthetic peptide
GGSLRY

SEQ ID NO: 164 - protein, 115 aa, Artificial Sequence
  Description of Artificial Sequence: Synthetic polypeptide
KVQLVESGGGLVQPGNSLRLSCAASGFTFSSFGMSWVRQAPGKGLEWVSSISGSGSDTLTADSVKGRFTISRDNSKNTLYLQMNSLRAEDTAVYYCTIGGSLVRSSQGTLVTVSS

SEQ ID NO: 165 - protein, 17 aa, Artificial Sequence
  Description of Artificial Sequence: Synthetic peptide
SISGSGSDTLTADSVKG

SEQ ID NO: 166 - protein, 8 aa, Artificial Sequence
  Description of Artificial Sequence: Synthetic peptide
TIGGSLVR

SEQ ID NO: 167 - protein, 6 aa, Artificial Sequence
  Description of Artificial Sequence: Synthetic peptide
GGSLVR

SEQ ID NO: 168 - protein, 115 aa, Artificial Sequence
  Description of Artificial Sequence: Synthetic polypeptide
KVQLVESGGGLVQPGNSLRLSCAASGFTFSSFGMGWVRQAPGKGLEWVSSISGSGSDTLYAPSVKGRFTISRDNSKNTLYLQMNSLRPEDTAVYYCTFAGSLSRSSQGTLVTVSS

SEQ ID NO: 169 - protein, 9 aa, Artificial Sequence
  Description of Artificial Sequence: Synthetic peptide
FTFSSFGMG

SEQ ID NO: 170 - protein, 5 aa, Artificial Sequence
  Description of Artificial Sequence: Synthetic peptide
SFGMG

SEQ ID NO: 171 - protein, 17 aa, Artificial Sequence
  Description of Artificial Sequence: Synthetic peptide
SISGSGSDTLYAPSVKG

SEQ ID NO: 172 - protein, 8 aa, Artificial Sequence
  Description of Artificial Sequence: Synthetic peptide
TFAGSLSR

SEQ ID NO: 173 - protein, 6 aa, Artificial Sequence
  Description of Artificial Sequence: Synthetic peptide
AGSLSR

SEQ ID NO: 174 - protein, 115 aa, Artificial Sequence
  Description of Artificial Sequence: Synthetic polypeptide
KVQLVESGGGLVQPGGSLRLSCAASGFTFSSFGMSWVRQAPGKGPEWVSSISGSGSDTLYADSVKGRFTISRDNSKNTLYLQMNSLRAEDTAVYYCTIGGSLRASSQGTLVTVSS

SEQ ID NO: 175 - protein, 115 aa, Artificial Sequence
  Description of Artificial Sequence: Synthetic polypeptide
KVQLVESGGGLVQPGNSLRLSCAASGFTFSSFGMSWVRQAPGKGLEWVSSISGSGSDTLYADSVKGRFTISRDNSKNTLYLQMNSLRAEDTAVYYCTIGGSLTRSSQGTLVTVSS

SEQ ID NO: 176 - protein, 8 aa, Artificial Sequence
  Description of Artificial Sequence: Synthetic peptide
TIGGSLTR

SEQ ID NO: 177 - protein, 6 aa, Artificial Sequence
  Description of Artificial Sequence: Synthetic peptide
GGSLTR

SEQ ID NO: 178 - protein, 115 aa, Artificial Sequence
  Description of Artificial Sequence: Synthetic polypeptide
KVQLVESGGGLVQPGNSLRLSCAASGFTFSSFGMSWVRQAPGKGLEWVSSISGGGSDTLYADSVRGRFTISRDNSKNTLYLQMNSLRAEDTAVYYCTIGGSLRASSQGTLVTVSS

SEQ ID NO: 179 - protein, 17 aa, Artificial Sequence
  Description of Artificial Sequence: Synthetic peptide
SISGGGSDTLYADSVRG

SEQ ID NO: 180 - protein, 115 aa, Artificial Sequence
  Description of Artificial Sequence: Synthetic polypeptide
KVQLVESGGGLVQPGNSLRLSCAASGFTFSSFGMSWVRQAPGKGLEWVSSISGSGSDALYADSAKGRFTISRDNAKTTLYLQMNSLRAEDTAVYYCTIGGSLSPSSQGTLVTVSS

SEQ ID NO: 181 - protein, 17 aa, Artificial Sequence
  Description of Artificial Sequence: Synthetic peptide
SISGSGSDALYADSAKG

SEQ ID NO: 182 - protein, 115 aa, Artificial Sequence
  Description of Artificial Sequence: Synthetic polypeptide
KVQLVESGGGLVQPGGSLRLSCAASGFTFSSFGMSWVRQAPGKGPEWVSSISGSGSDTLYADSVKGRFTISRDNSKNTLYLQMNSLRPEDTAVYYCTIGGSLKQSSQGTLVTVSS

SEQ ID NO: 183 - protein, 9 aa, Artificial Sequence
  Description of Artificial Sequence: Synthetic peptide
  MOD_RES (4)..(4): Ser, His, Gly or Pro
  MOD_RES (9)..(9): Ser, Thr or Gly
FTFXSFGMX

SEQ ID NO: 184 - protein, 5 aa, Artificial Sequence
  Description of Artificial Sequence: Synthetic peptide
  MOD_RES (5)..(5): Ser, Thr or Gly
SFGMX

SEQ ID NO: 185 - protein, 17 aa, Artificial Sequence
  Description of Artificial Sequence: Synthetic peptide
  MOD_RES (5)..(5): Ser or Gly
  MOD_RES (7)..(7): Ser, Ala or Gly
  MOD_RES (9)..(9): Thr or Ala
  MOD_RES (13)..(13): Asp or Pro
  MOD_RES (15)..(15): Val or Ala
  MOD_RES (16)..(16): Lys, Arg or Glu
SISGXGXDXLYAXSXXG

SEQ ID NO: 186 - protein, 8 aa, Artificial Sequence
  Description of Artificial Sequence: Synthetic peptide
  MOD_RES (2)..(2): Ile or Phe
  MOD_RES (3)..(3): Gly or Ala
  MOD_RES (6)..(6): Leu or Val
  MOD_RES (7)..(7): Ser, Arg, Lys or Thr
  MOD_RES (8)..(8): Arg, Pro, Ala, Gln or Lys
TXXGSXXX

SEQ ID NO: 187 - protein, 6 aa, Artificial Sequence
  Description of Artificial Sequence: Synthetic peptide
  MOD_RES (1)..(1): Gly or Ala
  MOD_RES (4)..(4): Leu or Val
  MOD_RES (5)..(5): Ser, Arg, Lys or Thr
  MOD_RES (6)..(6): Arg, Pro, Ala, Gln or Lys
XGSXXX

SEQ ID NO: 188 - protein, 9 aa, Artificial Sequence
  Description of Artificial Sequence: Synthetic peptide
  MOD_RES (4)..(4): Ser, His or Gly
  MOD_RES (9)..(9): Ser or Thr
FTFXSFGMX

SEQ ID NO: 189 - protein, 5 aa, Artificial Sequence
  Description of Artificial Sequence: Synthetic peptide
  MOD_RES (5)..(5): Ser or Thr
SFGMX

SEQ ID NO: 190 - protein, 17 aa, Artificial Sequence
  Description of Artificial Sequence: Synthetic peptide
  MOD_RES (5)..(5): Ser or Thr
  MOD_RES (16)..(16): Lys or Arg
SISGXGSDTLYADSVXG

SEQ ID NO: 191 - protein, 8 aa, Artificial Sequence
  Description of Artificial Sequence: Synthetic peptide
  MOD_RES (7)..(7): Ser, Arg or Thr
  MOD_RES (8)..(8): Arg, Pro, Tyr or Ala
TIGGSLXX

SEQ ID NO: 192 - protein, 6 aa, Artificial Sequence
  Description of Artificial Sequence: Synthetic peptide
  MOD_RES (5)..(5): Ser, Arg or Thr
  MOD_RES (6)..(6): Arg, Pro, Tyr or Ala
GGSLXX

SEQ ID NO: 193 - protein, 17 aa, Artificial Sequence
  Description of Artificial Sequence: Synthetic peptide
  MOD_RES (16)..(16): Lys or Arg
SISGSGSDTLYADSVXG

SEQ ID NO: 194 - protein, 8 aa, Artificial Sequence
  Description of Artificial Sequence: Synthetic peptide
  MOD_RES (7)..(7): Ser, Arg or Thr
  MOD_RES (8)..(8): Arg, Pro or Ala
TIGGSLXX

SEQ ID NO: 195 - protein, 6 aa, Artificial Sequence
  Description of Artificial Sequence: Synthetic peptide
  MOD_RES (5)..(5): Ser, Arg or Thr
  MOD_RES (6)..(6): Arg, Pro or Ala
GGSLXX

SEQ ID NO: 196 - skipped

SEQ ID NO: 197 - protein, 121 aa, Artificial Sequence
  Description of Artificial Sequence: Synthetic polypeptide
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 198 - protein, 112 aa, Artificial Sequence
  Description of Artificial Sequence: Synthetic polypeptide
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 199 - protein, 5 aa, Artificial Sequence
  Description of Artificial Sequence: Synthetic peptide
SYVMH

SEQ ID NO: 200 - protein, 14 aa, Artificial Sequence
  Description of Artificial Sequence: Synthetic peptide
WIGYINPYNDGTKY

SEQ ID NO: 201 - protein, 12 aa, Artificial Sequence
  Description of Artificial Sequence: Synthetic peptide
GTYYYGTRVFDY

SEQ ID NO: 202 - protein, 16 aa, Artificial Sequence
  Description of Artificial Sequence: Synthetic peptide
RSSKSLQNVNGNTYLY

SEQ ID NO: 203 - protein, 7 aa, Artificial Sequence
  Description of Artificial Sequence: Synthetic peptide
RMSNLNS

SEQ ID NO: 204 - protein, 9 aa, Artificial Sequence
  Description of Artificial Sequence: Synthetic peptide
MQHLEYPIT

SEQ ID NO: 205 - protein, 124 aa, Artificial Sequence
  Description of Artificial Sequence: Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 206 - protein, 111 aa, Artificial Sequence
  Description of Artificial Sequence: Synthetic polypeptide
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 207 - protein, 5 aa, Artificial Sequence
  Description of Artificial Sequence: Synthetic peptide
SYWMN

SEQ ID NO: 208 - protein, 17 aa, Artificial Sequence
  Description of Artificial Sequence: Synthetic peptide
QIWPGDGDTNYNGKFKG

SEQ ID NO: 209 - protein, 15 aa, Artificial Sequence
  Description of Artificial Sequence: Synthetic peptide
RETTTVGRYYYAMDY

SEQ ID NO: 210 - protein, 15 aa, Artificial Sequence
  Description of Artificial Sequence: Synthetic peptide
KASQSVDYDGDSYLN

SEQ ID NO: 211 - protein, 7 aa, Artificial Sequence
  Description of Artificial Sequence: Synthetic peptide
DASNLVS

SEQ ID NO: 212 - protein, 9 aa, Artificial Sequence
  Description of Artificial Sequence: Synthetic peptide
QQSTEDPWT

SEQ ID NO: 213 - protein, 124 aa, Artificial Sequence
  Description of Artificial Sequence: Synthetic polypeptide
QVQLQQSGAEVKKPGSSVKVSCKASGYAFSSYWMNWVRQRPGQGLEWIGQIWPGDGDTNYNGKFKGRATITADESTNTAYMELSSLRSEDTAFYSCARRETTTVGRYYYAMDYWGQGTTVTVSS

SEQ ID NO: 214 - protein, 112 aa, Artificial Sequence
  Description of Artificial Sequence: Synthetic polypeptide
DIQLTQSPSSLSASVGDRVTITCKASQSVDYDGDSYLNWYQQIPGKAPKLLIYDASNLVSGIPPRFSGSGSGTDYTFTISSLQPEDIATYHCQQSTEDPWTFGGGTKLQIKR

SEQ ID NO: 215 - protein, 17 aa, Artificial Sequence
  Description of Artificial Sequence: Synthetic peptide
QIWPGDGDTNYNGKFKG

SEQ ID NO: 216 - protein, 15 aa, Artificial Sequence
  Description of Artificial Sequence: Synthetic peptide
RETTTVGRYYYAMDY

SEQ ID NO: 217 - protein, 15 aa, Artificial Sequence
  Description of Artificial Sequence: Synthetic peptide
KASQSVDYDGDSYLN

SEQ ID NO: 218 - protein, 7 aa, Artificial Sequence
  Description of Artificial Sequence: Synthetic peptide
DASNLVS

SEQ ID NO: 219 - protein, 9 aa, Artificial Sequence
  Description of Artificial Sequence: Synthetic peptide
QQSTEDPWT

SEQ ID NO: 220 - protein, 120 aa, Artificial Sequence
  Description of Artificial Sequence: Synthetic polypeptide
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 221 - protein, 104 aa, Artificial Sequence
  Description of Artificial Sequence: Synthetic polypeptide
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 222 - protein, 5 aa, Artificial Sequence
  Description of Artificial Sequence: Synthetic peptide
SNWMH

SEQ ID NO: 223 - protein, 10 aa, Artificial Sequence
  Description of Artificial Sequence: Synthetic peptide
EIDPSDSYTN

SEQ ID NO: 224 - protein, 11 aa, Artificial Sequence
  Description of Artificial Sequence: Synthetic peptide
GSNPYYYAMDY

SEQ ID NO: 225 - protein, 10 aa, Artificial Sequence
  Description of Artificial Sequence: Synthetic peptide
SASSGVNYMH

SEQ ID NO: 226 - protein, 7 aa, Artificial Sequence
  Description of Artificial Sequence: Synthetic peptide
DTDKTAS

SEQ ID NO: 227 - protein, 7 aa, Artificial Sequence
  Description of Artificial Sequence: Synthetic peptide
HQRGSYT

SEQ ID NO: 228 - protein, 121 aa, Artificial Sequence
  Description of Artificial Sequence: Synthetic polypeptide
EVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSS

SEQ ID NO: 229 - protein, 107 aa, Artificial Sequence
  Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 230 - protein, 5 aa, Artificial Sequence
  Description of Artificial Sequence: Synthetic peptide
SSWIG

SEQ ID NO: 231 - protein, 17 aa, Artificial Sequence
  Description of Artificial Sequence: Synthetic peptide
IIYPDDSDTRYSPSFQG

SEQ ID NO: 232 - protein, 12 aa, Artificial Sequence
  Description of Artificial Sequence: Synthetic peptide
HVTMIWGVIIDF

SEQ ID NO: 233 - protein, 11 aa, Artificial Sequence
  Description of Artificial Sequence: Synthetic peptide
RASQGISSALA

SEQ ID NO: 234 - protein, 7 aa, Artificial Sequence
  Description of Artificial Sequence: Synthetic peptide
DASSLES

SEQ ID NO: 235 - protein, 9 aa, Artificial Sequence
  Description of Artificial Sequence: Synthetic peptide
QQFNSYPFT

SEQ ID NO: 236 - protein, 121 aa, Artificial Sequence; identical to SEQ ID NO: 228
  Description of Artificial Sequence: Synthetic polypeptide

SEQ ID NO: 237 - protein, 107 aa, Artificial Sequence
  Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 238 - protein, 5 aa, Artificial Sequence
  Description of Artificial Sequence: Synthetic peptide
SSWIG

SEQ ID NO: 239 - protein, 17 aa, Artificial Sequence
  Description of Artificial Sequence: Synthetic peptide
IIYPDDSDTRYSPSFQG

SEQ ID NO: 240 - protein, 12 aa, Artificial Sequence
  Description of Artificial Sequence: Synthetic peptide
HVTMIWGVIIDF

SEQ ID NO: 241 - protein, 11 aa, Artificial Sequence
  Description of Artificial Sequence: Synthetic peptide
RASQGISSALA

SEQ ID NO: 242 - protein, 7 aa, Artificial Sequence
  Description of Artificial Sequence: Synthetic peptide
DASSLES

SEQ ID NO: 243 - protein, 9 aa, Artificial Sequence
  Description of Artificial Sequence: Synthetic peptide
QQFNSYPYT

SEQ ID NO: 244 - protein, 119 aa, Artificial Sequence
  Description of Artificial Sequence: Synthetic polypeptide
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 245 - protein, 108 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 246 - protein, 5 aa, Artificial Sequence
  Description of Artificial Sequence: Synthetic peptide
SYAIS

SEQ ID NO: 247 - protein, 17 aa, Artificial Sequence
  Description of Artificial Sequence: Synthetic peptide
GIIPIFGTTNYAQQFQG

SEQ ID NO: 248 - protein, 10 aa, Artificial Sequence
  Description of Artificial Sequence: Synthetic peptide
EAVAADWLDP

SEQ ID NO: 249 - protein, 12 aa, Artificial Sequence
  Description of Artificial Sequence: Synthetic peptide
RASQSVSSSYLA

SEQ ID NO: 250 - protein, 7 aa, Artificial Sequence
  Description of Artificial Sequence: Synthetic peptide
GASSRAT

SEQ ID NO: 251 - protein, 9 aa, Artificial Sequence
  Description of Artificial Sequence: Synthetic peptide
QQYGSSRFT

SEQ ID NO: 252 - protein, 124 aa, Artificial Sequence
  Description of Artificial Sequence: Synthetic polypeptide
EVQLVQSGAEVKKPGESLKISCKGSGYSFTSYWIAWVRQMPGKGLEWMGIIYPGDSDTRYSPSFQGQVTISADKSISTAYLQWSSLKASDTAMYYCARQGYSSGWDSYYGMGVWGQGTTVTVSS

SEQ ID NO: 253 - protein, 107 aa, Artificial Sequence; identical to SEQ ID NO: 237
  Description of Artificial Sequence: Synthetic polypeptide

SEQ ID NO: 254 - protein, 5 aa, Artificial Sequence
  Description of Artificial Sequence: Synthetic peptide
SYWIA

SEQ ID NO: 255 - protein, 17 aa, Artificial Sequence
  Description of Artificial Sequence: Synthetic peptide
IIYPGDSDTRYSPSFQG

SEQ ID NO: 256 - protein, 15 aa, Artificial Sequence
  Description of Artificial Sequence: Synthetic peptide
QGYSSGWDSYYGMGV

SEQ ID NO: 257 - protein, 11 aa, Artificial Sequence
  Description of Artificial Sequence: Synthetic peptide
RASQGISSALA

SEQ ID NO: 258 - protein, 7 aa, Artificial Sequence
  Description of Artificial Sequence: Synthetic peptide
DASSLES

SEQ ID NO: 259 - protein, 9 aa, Artificial Sequence
  Description of Artificial Sequence: Synthetic peptide
QQFNSYPYT

SEQ ID NO: 260 - protein, 123 aa, Artificial Sequence
  Description of Artificial Sequence: Synthetic polypeptide
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSS

SEQ ID NO: 261 - protein, 107 aa, Artificial Sequence
  Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIK

SEQ ID NO: 262 - protein, 5 aa, Artificial Sequence
  Description of Artificial Sequence: Synthetic peptide
SYTIN

SEQ ID NO: 263 - protein, 17 aa, Artificial Sequence
  Description of Artificial Sequence: Synthetic peptide
GIIPIFGIPNYAQKFQG

SEQ ID NO: 264 - protein, 14 aa, Artificial Sequence
  Description of Artificial Sequence: Synthetic peptide
ASGGSADYSYGMDV

SEQ ID NO: 265 - protein, 11 aa, Artificial Sequence
  Description of Artificial Sequence: Synthetic peptide
RASQGISSWLA

SEQ ID NO: 266 - protein, 7 aa, Artificial Sequence
  Description of Artificial Sequence: Synthetic peptide
AASSLQS

SEQ ID NO: 267 - protein, 9 aa, Artificial Sequence
  Description of Artificial Sequence: Synthetic peptide
QQYKRYPYT

SEQ ID NO: 268 - protein, 121 aa, Artificial Sequence
  Description of Artificial Sequence: Synthetic polypeptide
EVQLVQSGAEVKKPGESLNISCKGSGYSFTSYWIGWVRQMPGKGLEWMGIIYPGDSDTRYSPSFQGQVTISADKSINTAYLQWSSLKASDTAMYYCARGVSMIWGVIMDVWGQGTTVTVSS

SEQ ID NO: 269 - protein, 107 aa, Artificial Sequence
  Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPWTFGQGTKVEIK

SEQ ID NO: 270 - protein, 5 aa, Artificial Sequence
  Description of Artificial Sequence: Synthetic peptide
SYWIG

SEQ ID NO: 271 - protein, 17 aa, Artificial Sequence
  Description of Artificial Sequence: Synthetic peptide
IIYPGDSDTRYSPSFQG

SEQ ID NO: 272 - protein, 12 aa, Artificial Sequence
  Description of Artificial Sequence: Synthetic peptide
GVSMIWGVIMDV

SEQ ID NO: 273 - protein, 11 aa, Artificial Sequence
  Description of Artificial Sequence: Synthetic peptide
RASQGISSALA

SEQ ID NO: 274 - protein, 7 aa, Artificial Sequence
  Description of Artificial Sequence: Synthetic peptide
DASSLES

SEQ ID NO: 275 - protein, 9 aa, Artificial Sequence
  Description of Artificial Sequence: Synthetic peptide
QQFNSYPWT

SEQ ID NO: 276 - protein, 124 aa, Artificial Sequence
  Description of Artificial Sequence: Synthetic polypeptide
EVQLVQSGAEVKKPGESLQISCKGSGYTFTNYWIAWVRQMPGKGLEWMGIIYPGDSDTRYSPSFQGQVTISADKSISTAYLQWSGLKASDTAMYYCARQGYSSGWRSYYGMGVWGQGTTVTVSS

SEQ ID NO: 277 - protein, 107 aa, Artificial Sequence
  Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPHTFGQGTKLEIK

SEQ ID NO: 278 - protein, 5 aa, Artificial Sequence
  Description of Artificial Sequence: Synthetic peptide
NYWIA

SEQ ID NO: 279 - protein, 17 aa, Artificial Sequence
  Description of Artificial Sequence: Synthetic peptide
IIYPGDSDTRYSPSFQG

SEQ ID NO: 280 - protein, 15 aa, Artificial Sequence
  Description of Artificial Sequence: Synthetic peptide
QGYSSGWRSYYGMGV

SEQ ID NO: 281 - protein, 11 aa, Artificial Sequence
  Description of Artificial Sequence: Synthetic peptide
RASQGISSALA

SEQ ID NO: 282 - protein, 45 aa, Artificial Sequence
  Description of Artificial Sequence: Synthetic polypeptide
LKEAKEKAIEELKKAGITSDYYFDLINKAKTVEGVNALKDEILKA

SEQ ID NO: 283 - protein, 9 aa, Artificial Sequence
  Description of Artificial Sequence: Synthetic peptide
QQFNSYPHT

SEQ ID NO: 284 - protein, 124 aa, Artificial Sequence; identical to SEQ ID NO: 276
  Description of Artificial Sequence: Synthetic polypeptide

SEQ ID NO: 285 - protein, 107 aa, Artificial Sequence; identical to SEQ ID NO: 277
  Description of Artificial Sequence: Synthetic polypeptide

SEQ ID NO: 286 - protein, 5 aa, Artificial Sequence
  Description of Artificial Sequence: Synthetic peptide
NYWIA

SEQ ID NO: 287 - protein, 17 aa, Artificial Sequence
  Description of Artificial Sequence: Synthetic peptide
IIYPGDSDTRYSPSFQG

SEQ ID NO: 288 - protein, 15 aa, Artificial Sequence
  Description of Artificial Sequence: Synthetic peptide
QGYSSGWRSYYGMGV

SEQ ID NO: 289 - protein, 11 aa, Artificial Sequence
  Description of Artificial Sequence: Synthetic peptide
RASQGISSALA

SEQ ID NO: 290 - protein, 7 aa, Artificial Sequence
  Description of Artificial Sequence: Synthetic peptide
DASSLES

SEQ ID NO: 291 - protein, 9 aa, Artificial Sequence
  Description of Artificial Sequence: Synthetic peptide
QQFNSYPHT

SEQ ID NO: 292 - protein, 20 aa, Artificial Sequence
  Description of Artificial Sequence: Synthetic peptide
GSGSGSGSGSGSGSGSGSGS

SEQ ID NO: 293 - protein, 30 aa, Artificial Sequence
  Description of Artificial Sequence: Synthetic polypeptide
GGSGGSGGSGGSGGSGGSGGSGGSGGSGGS

SEQ ID NO: 294 - protein, 40 aa, Artificial Sequence
  Description of Artificial Sequence: Synthetic polypeptide
GGGSGGGSGGGSGGGSGGGSGGGSGGGSGGGSGGGSGGGS

SEQ ID NO: 295 - protein, 40 aa, Artificial Sequence
  Description of Artificial Sequence: Synthetic polypeptide
GGSGGGSGGGSGGGSGGGSGGGSGGGSGGGSGGGSGGGSG

SEQ ID NO: 296 - protein, 50 aa, Artificial Sequence
  Description of Artificial Sequence: Synthetic polypeptide
GGSGGGGSGGGGSGGGGSGGGGSGGGGSGGGGSGGGGSGGGGSGGGGSGG

SEQ ID NO: 297 - protein, 50 aa, Artificial Sequence
  Description of Artificial Sequence: Synthetic polypeptide
GGGGSGGGGSGGGGSGGGGSGGGGSGGGGSGGGGSGGGGSGGGGSGGGGS

SEQ ID NO: 298 - protein, 20 aa, Artificial Sequence
  Description of Artificial Sequence: Synthetic peptide
GGGGSGGGGSGGGGSGGGGS

SEQ ID NO: 299 - protein, 15 aa, Artificial Sequence
  Description of Artificial Sequence: Synthetic peptide
GGGGSGGGGSGGGGS

SEQ ID NO: 300 - protein, 100 aa, Artificial Sequence
  Description of Artificial Sequence: Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 301 - protein, 100 aa, Artificial Sequence
  Description of Artificial Sequence: Synthetic polypeptide
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 302 - protein, 9 aa, Artificial Sequence
  Description of Artificial Sequence: Synthetic peptide
GGGGSGGGS

SEQ ID NO: 303 - protein, 642 aa, Artificial Sequence
  Description of Artificial Sequence: Synthetic polypeptide
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 304 - protein, 642 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 305 - protein, 642 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKHHHHHHHHHH

SEQ ID NO: 306 - protein, 642 aa, Artificial Sequence
  Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKHHHHHHHHHH

SEQ ID NO: 307 - protein, 642 aa, Artificial Sequence
  Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSHHHHHHHHHH

SEQ ID NO: 308 - protein, 636 aa, Artificial Sequence
  Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 309 - protein, 636 aa, Artificial Sequence
  Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSHHHHHHHHHH

SEQ ID NO: 310 - protein, 636 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 311 - protein, 636 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRHHHHHHHHHH

SEQ ID NO: 312 - protein, 636 aa, Artificial Sequence
  Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRHHHHHHHHHH

SEQ ID NO: 313 - protein, 636 aa, Artificial Sequence
  Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSHHHHHHHHHH

SEQ ID NO: 314 - protein, 634 aa, Artificial Sequence
  Description of Artificial Sequence: Synthetic polypeptide
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRHHHHHHHHHH

SEQ ID NO: 315 - protein, 122 aa, Artificial Sequence
  Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGVSGIYNLHGFDIWGQGTLVTVSS

SEQ ID NO: 316 - protein, 108 aa, Artificial Sequence
  Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQGRFGSPFTFGQGTKVEIK

SEQ ID NO: 317 - protein, 8 aa, Artificial Sequence
  Description of Artificial Sequence: Synthetic peptide
GYSFTSYW

SEQ ID NO: 318 - protein, 8 aa, Artificial Sequence
  Description of Artificial Sequence: Synthetic peptide
IYPGDSDT

SEQ ID NO: 319 - protein, 15 aa, Artificial Sequence
  Description of Artificial Sequence: Synthetic peptide
ARGVSGIYNLHGFDI

SEQ ID NO: 320 - protein, 6 aa, Artificial Sequence
  Description of Artificial Sequence: Synthetic peptide
QSISSY

SEQ ID NO: 321 - protein, 3 aa, Artificial Sequence
  Description of Artificial Sequence: Synthetic peptide
AAS

SEQ ID NO: 322 - protein, 10 aa, Artificial Sequence
  Description of Artificial Sequence: Synthetic peptide
QQGRFGSPFT

SEQ ID NO: 323 - protein, 117 aa, Artificial Sequence
  Description of Artificial Sequence: Synthetic polypeptide
QVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARYYYSRLDYWGQGTLVTVSS

SEQ ID NO: 324 - protein, 113 aa, Artificial Sequence
  Description of Artificial Sequence: Synthetic polypeptide
QTVVTQEPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGTGTKLTVLGSR

SEQ ID NO: 325 - protein, 117 aa, Artificial Sequence
  Description of Artificial Sequence: Synthetic polypeptide
QVQLVESGGGLVQPGGSLRLSCAASGFTFSSYAMSWVRQAPGKGLEWVSGISASGGSTYYADSVKGRFTISRDNSKNTLYLQMNSLRAEDTAVYYCARYYLSQIDSWGQGTLVTVSS

SEQ ID NO: 326 - protein, 110 aa, Artificial Sequence
  Description of Artificial Sequence: Synthetic polypeptide
QSVLTQPPSVSAAPGQKVTISCSGSSSNIGNNYVSWYRQLPGTAPKLLIYENNKRPSGIPDRFSGSKSGTSATLGITGLQTGDEADYYCGTWDSSLRAGVFGTGTKVTVL

SEQ ID NO: 327 - protein, 118 aa, Artificial Sequence
  Description of Artificial Sequence: Synthetic polypeptide
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATGIYSRPLGYWGQGTLVTVSS

SEQ ID NO: 328 - protein, 111 aa, Artificial Sequence
  Description of Artificial Sequence: Synthetic polypeptide
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGHVVFGGGTKLTVL

SEQ ID NO: 329 - protein, 121 aa, Artificial Sequence
  Description of Artificial Sequence: Synthetic polypeptide
EVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARSDGKHFWQQYDAWGQGTLVTVSS

SEQ ID NO: 330 - protein, 110 aa, Artificial Sequence
  Description of Artificial Sequence: Synthetic polypeptide
SYVLTQPPSASGTPGQRVTISCSGSSSNIGSHTVNWYQQLPGTAPKLLIYSNNQRPSGVPDRFSGSKSGTSASLAISGLQSEDEADYYCAAWDDSLNGYVFGTGTKVTVL

SEQ ID NO: 331 - protein, 114 aa, Artificial Sequence
  Description of Artificial Sequence: Synthetic polypeptide
EVQLVESGGGLVQPGGSLRLSCAASGFTVSSNYMSWVRQAPGKGLEWVSAISGSGGSTYYADSVKGRFTISRDNSKNTLYLQMNSLRAEDTAVYYCARMNIDYWGQGTLVTVSS

SEQ ID NO: 332 - protein, 107 aa, Artificial Sequence
  Description of Artificial Sequence: Synthetic polypeptide
DIQLTQSPSSLSAYVGDRVTITCRASQGITNSLAWYQQKPGKAPKLLLHAASRLESGVPSRFSGSGFGTDFTLTISSLQPEDFAVYYCQHYLGTPYSFGQGTKVEIK

SEQ ID NO: 333 - protein, 124 aa, Artificial Sequence
  Description of Artificial Sequence: Synthetic polypeptide
EVQLVQSGAEVKRPGESLTISCKGSEYSFASYWITWVRQMPGKGLEWMGRIDPSDSYTNYSPSFQGHVTISADKSISTAYLQWSSLKASDTAIYYCARPFQYDYGGYSDAFDIWGQGTMVTVSS

SEQ ID NO: 334 - protein, 107 aa, Artificial Sequence
  Description of Artificial Sequence: Synthetic polypeptide
EIVLTQSPSSLSASVGDRVTISCRASQSVSRFLNWYQQKPGKAPKLLIYGVSTLERGVPSRFSGSGSGTDFTLTISSLQPEDFATYYCQESYIIPLTFGGGTKLEIK

SEQ ID NO: 335 - protein, 118 aa, Artificial Sequence
  Description of Artificial Sequence: Synthetic polypeptide
QMQLVQSGAEVKKAGSSVKVSCETSGGTFSSSSVNWVRQAPGQGLEWMGGIIPIVGTPNYAQKFQDRVTITAVESTFTAYMELSGLRSEDTAVYYCARGGYRDYMDVWGRGTTVTVSS

SEQ ID NO: 336 - protein, 111 aa, Artificial Sequence
  Description of Artificial Sequence: Synthetic polypeptide
EIVMTQSPLSLSVTPGEPASISCRSSQSLLDSNGFNSLDWYLQKPGQSPQLLIHLGSDRASGVPDRFSGSGSGTDFTLKISRVEAEDVGIYYCMQSLQIPTFGQGTKVEIK

SEQ ID NO: 337 - protein, 117 aa, Artificial Sequence
  Description of Artificial Sequence: Synthetic polypeptide
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGGFGAEFDYWGQGTLVTVSS

SEQ ID NO: 338 - protein, 110 aa, Artificial Sequence
  Description of Artificial Sequence: Synthetic polypeptide
SYELTQPPSASGTPGQRVTISCSGSSSNIGSNYVYWYQQLPGTAPKLLIYRNNQRPSGVPDRFSGSKSGTSASLAISGLRSEDEADYYCAAWDDSLSGYVFGTGTKVTVL

SEQ ID NO: 339 - protein, 123 aa, Artificial Sequence
  Description of Artificial Sequence: Synthetic polypeptide
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGGISDDYYGSGSYDNWGQGTLVTVSS

SEQ ID NO: 340 - protein, 107 aa, Artificial Sequence
  Description of Artificial Sequence: Synthetic polypeptide
SYVLTQPPSVSVSPGQTASITCSGDKLGDKYASWYQQKPGQSPVLVIYQDNKRPSGIPERFSGSNSGNTATLTISGTQAMDEADYYCQAWDSSTEDVFGPGTKVTVL

SEQ ID NO: 341 - protein, 118 aa, Artificial Sequence
  Description of Artificial Sequence: Synthetic polypeptide
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARERGMGYAFDIWGQGTMVTVSS

SEQ ID NO: 342 - protein, 107 aa, Artificial Sequence
  Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPFTFGGGTKVEIK

SEQ ID NO: 343 - protein, 114 aa, Artificial Sequence
  Description of Artificial Sequence: Synthetic polypeptide
QLQLQESGPGLVKPSETLSLTCSVSGVSMSENYWSWIRQPPGKRLEWIGCAHYTGDTHYNPSLKGRVTISLDTSMNQFSLRLNSVTAADTAVYYCASYHPFNYWGQGTLVTVSS

SEQ ID NO: 344 - protein, 104 aa, Artificial Sequence
  Description of Artificial Sequence: Synthetic polypeptide
DIQMTQSPSSLSASVGDRVTITCRASQGIGSYLAWYQQKPGKAPKLLIYPASTLQSGVPSRFSGSGSGTEFTLTISSLQPEDFATYYCQQLNSLFGQGTRLEIK

SEQ ID NO: 345 - protein, 126 aa, Artificial Sequence
  Description of Artificial Sequence: Synthetic polypeptide
EVQLVQSGAEVRRPGATVKISCKVSGYTFNDFYLHWVRQAPGKGLEWMGRIDPEDGKTRYAEKFQGRLTITADTSTDTLYMQLGGLTSDDTAVYYCTTDWGYSSSLREEDIWYDCWGQGTLVTVSS

SEQ ID NO: 346 - protein, 110 aa, Artificial Sequence
  Description of Artificial Sequence: Synthetic polypeptide
QAVLTQPPSASGTPGQRVTISCSGSSSNIGTKTVNWYQVLPGTAPKLLIYSNYRRPSGVPDRFSGSKSGTSASLAISGLQSDDEADYYCALWDDSLDGYVFGTGTKVTVL

SEQ ID NO: 347 - protein, 122 aa, Artificial Sequence
  Description of Artificial Sequence: Synthetic polypeptide
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDYGYGDYGDAFDIWGQGTMVTVSS

SEQ ID NO: 348 - protein, 108 aa, Artificial Sequence
  Description of Artificial Sequence: Synthetic polypeptide
SYELTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVL

SEQ ID NO: 349 - protein, 121 aa, Artificial Sequence
  Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVVGTIYSMQYDVWGQGTLVTVSS

SEQ ID NO: 350 - protein, 108 aa, Artificial Sequence
  Description of Artificial Sequence: Synthetic polypeptide
SYVLTQPPSVSVAPGKTARITCGGNNIGSKSVHWYQQRPGQAPVLVVYDDSDRPSGIPERFSGSNSGNTATLTISRVEAGDEADYSCQVWDSSSDHYVFGPGTKVTVL

SEQ ID NO: 351 - protein, 130 aa, Artificial Sequence
  Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNMDSWGQGTLVTVSS

SEQ ID NO: 352 - protein, 108 aa, Artificial Sequence
  Description of Artificial Sequence: Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVL

SEQ ID NO: 353 - protein, 121 aa, Artificial Sequence
  Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWSSTWDSMYMDYWGQGTLVTVSS

SEQ ID NO: 354 - protein, 110 aa, Artificial Sequence
  Description of Artificial Sequence: Synthetic polypeptide
QAVLTQPPSVSEAPRQRVTISCSGSSSNVGNNAVNWYQQVPGKAPKLLIYYDDLLSSGVSDRFSGSKSGTSASLAISGLQSEDEADYYCAAWDDSLNGPVFGGGTKLTVL

SEQ ID NO: 355 - protein, 121 aa, Artificial Sequence
  Description of Artificial Sequence: Synthetic polypeptide
EVQLVQSGAEVKKPGESLRISCKGSGYSFTSYWIGWVRQMPGKGLEWMGIIYPGDSDTRYSPSFQGQVTISADKSISTAYLQWSSLKASDTAMYYCARVTYSMDSYYFDSWGQGTLVTVSS

SEQ ID NO: 356 - protein, 108 aa, Artificial Sequence
  Description of Artificial Sequence: Synthetic polypeptide
QPVLTQPPSVSVAPGKTARITCGGNNIGSESVHWYQQKPGQAPMVVIYYDSNRPSGIPERFSGSNSGNTATLTVSRVEAEDEADYYCQVWNSSSDHRGVFGGGTKLTV

SEQ ID NO: 357 - protein, 121 aa, Artificial Sequence
  Description of Artificial Sequence: Synthetic polypeptide
EVQLQQSGPELVKPGASVKMSCKASGYTFTNYVIHWVKQKPGQGLEWIGYFNPYNDGTEYNEKFKAKATLTSDKSSSTAYMELSSLTSEDSAVYYCAKGPYYYGSSPFDYWGQGTTLTVSS

SEQ ID NO: 358 - protein, 112 aa, Artificial Sequence
  Description of Artificial Sequence: Synthetic polypeptide
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPYTFGGGTKLEIK

SEQ ID NO: 359 - protein, 10 aa, Artificial Sequence
  Description of Artificial Sequence: Synthetic peptide
GYTFTNYVIH

SEQ ID NO: 360 - protein, 17 aa, Artificial Sequence
  Description of Artificial Sequence: Synthetic peptide
YFNPYNDGTEYNEKFKA

SEQ ID NO: 361 - protein, 12 aa, Artificial Sequence
  Description of Artificial Sequence: Synthetic peptide
GPYYYGSSPFDY

SEQ ID NO: 362 - protein, 16 aa, Artificial Sequence
  Description of Artificial Sequence: Synthetic peptide
RSSQSLENSNGNTYLN

SEQ ID NO: 363 - protein, 7 aa, Artificial Sequence
  Description of Artificial Sequence: Synthetic peptide
RVSNRFS

SEQ ID NO: 364 - protein, 7 aa, Artificial Sequence
  Description of Artificial Sequence: Synthetic peptide
RVSNRFS

SEQ ID NO: 365 - protein, 121 aa, Artificial Sequence
  Description of Artificial Sequence: Synthetic polypeptide
QVQLQQSGAELVRPGSSVKISCKASGYAFSTYWMNWVKQRPGQGLEWIGQIYPGDDDTKYNGKFKGKASLTADKSSSTAYMQLISLTSEDSAVYFCARRYFRYDYWYSDVWGAGTTVTVTS

SEQ ID NO: 366 - protein, 107 aa, Artificial Sequence
  Description of Artificial Sequence: Synthetic polypeptide
DIQMTQTTSSLSASLGDRVTISCRASQDISNYLNWYQQKPDGTVKLLIYYTSRLHSGVPARFSGSGSGTDYSLTISNLEQEDIATYFCHQGNTLPLTFGAGTKLELK

SEQ ID NO: 367 - protein, 10 aa, Artificial Sequence
  Description of Artificial Sequence: Synthetic peptide
GYAFSTYWMN

SEQ ID NO: 368 - protein, 17 aa, Artificial Sequence
  Description of Artificial Sequence: Synthetic peptide
QIYPGDDDTKYNGKFKG

SEQ ID NO: 369 - protein, 12 aa, Artificial Sequence
  Description of Artificial Sequence: Synthetic peptide
RYFRYDYWYSDV

SEQ ID NO: 370 - protein, 11 aa, Artificial Sequence
  Description of Artificial Sequence: Synthetic peptide
RASQDISNYLN

SEQ ID NO: 371 - protein, 7 aa, Artificial Sequence
  Description of Artificial Sequence: Synthetic peptide
YTSRLHS

SEQ ID NO: 372 - protein, 9 aa, Artificial Sequence
  Description of Artificial Sequence: Synthetic peptide
HQGNTLPLT

SEQ ID NO: 373 - protein, 116 aa, Artificial Sequence
  Description of Artificial Sequence: Synthetic polypeptide
EIQLQQSGPELVKPGASVKVSCKASGYAFTSYNMYWVKQSHGKSLEWIGYIDPYNGDTTYNQKFKGKATLTVDKSSSTAYMHLNSLTSEDSAVYYCLTTAYAMDYWGQGTSVTVSS

SEQ ID NO: 374 - protein, 106 aa, Artificial Sequence
  Description of Artificial Sequence: Synthetic polypeptide
QIVLTQSPAIMSASLGEEITLTCSASSTVNYMHWYQQKSGTSPKLLIYSTSNLASGVPSRFSGSGSGTFYSLTIRSVEAEDAADYYCHQWSSYPYTFGGGTKLEIK

SEQ ID NO: 375 - protein, 10 aa, Artificial Sequence
  Description of Artificial Sequence: Synthetic peptide
GYAFTSYNMY

SEQ ID NO: 376 - protein, 17 aa, Artificial Sequence
  Description of Artificial Sequence: Synthetic peptide
YIDPYNGDTTYNQKFKG

SEQ ID NO: 377 - protein, 7 aa, Artificial Sequence
  Description of Artificial Sequence: Synthetic peptide
TAYAMDY

SEQ ID NO: 378 - protein, 10 aa, Artificial Sequence
  Description of Artificial Sequence: Synthetic peptide
SASSTVNYMH

SEQ ID NO: 379 - protein, 7 aa, Artificial Sequence
  Description of Artificial Sequence: Synthetic peptide
STSNLAS

SEQ ID NO: 380 - protein, 9 aa, Artificial Sequence
  Description of Artificial Sequence: Synthetic peptide
HQWSSYPYT

SEQ ID NO: 381 - protein, 121 aa, Artificial Sequence
  Description of Artificial Sequence: Synthetic polypeptide
QVQLVQSGAEVKKPGSSVKVSCKASGYTFTDYVIHWVRQAPGQGLEWMGYFNPYNDGTEYNEKFKARVTITADKSTSTAYMELSSLRSEDTAVYYCARGPYYYGSSPFDYWGQGTTVTVSS

SEQ ID NO: 382 - protein, 112 aa, Artificial Sequence
  Description of Artificial Sequence: Synthetic polypeptide
DIVMTQTPLSLPVTPGEPASISCRSSQSLENSNHNTYINWYLQKPGQSPQLLIYRVSKRFSGVPDRFSGSGSGTDFTLKISRVEAEDVGVYYCHQVTHVPYTFGQGTKLEIK

SEQ ID NO: 383 - protein, 10 aa, Artificial Sequence
  Description of Artificial Sequence: Synthetic peptide
GYTFTNYVIH

SEQ ID NO: 384 - protein, 17 aa, Artificial Sequence
  Description of Artificial Sequence: Synthetic peptide
YFNPYNDGTEYNEKFKA

SEQ ID NO: 385 - protein, 12 aa, Artificial Sequence
  Description of Artificial Sequence: Synthetic peptide
GPYYYGSSPFDY

SEQ ID NO: 386 - protein, 16 aa, Artificial Sequence
  Description of Artificial Sequence: Synthetic peptide
RSSQSLENSNHNTYIN

SEQ ID NO: 387 - protein, 9 aa, Artificial Sequence
  Description of Artificial Sequence: Synthetic peptide
HQVTHVPYT

SEQ ID NO: 388 - protein, 121 aa, Artificial Sequence
  Description of Artificial Sequence: Synthetic polypeptide
QVQLVQSGAEVKKPGASVKVSCKASGYAFSTYWMNWVRQAPGQGLEWMGQIYPGDDDTKYSGKFKGRVTITADKSTSTAYMELSSLRSEDTAVYYCARRYFRYDYWYSDVWGQGTTVTVSS

SEQ ID NO: 389 - protein, 107 aa, Artificial Sequence
  Description of Artificial Sequence: Synthetic polypeptide
DIQMTQSPSSLSASVGDRVTITCRASQDISNYLNWYQQKPGKVPKLLIYYTSRLHSGVPSRFSGSGSGTDFTLTISSLQPEDVATYYCHQGNTLPLTFGQGTKLEIK

SEQ ID NO: 390 - protein, 10 aa, Artificial Sequence
  Description of Artificial Sequence: Synthetic peptide
GYAFSTYWMN

SEQ ID NO: 391 - protein, 17 aa, Artificial Sequence
  Description of Artificial Sequence: Synthetic peptide
QIYPGDDDTKYSGKFKG

SEQ ID NO: 392 - protein, 12 aa, Artificial Sequence
  Description of Artificial Sequence: Synthetic peptide
RYFRYDYWYSDV

SEQ ID NO: 393 - protein, 11 aa, Artificial Sequence
  Description of Artificial Sequence: Synthetic peptide
RASQDISNYLN

SEQ ID NO: 394 - protein, 7 aa, Artificial Sequence
  Description of Artificial Sequence: Synthetic peptide
YTSRLHS

SEQ ID NO: 395 - protein, 9 aa, Artificial Sequence
  Description of Artificial Sequence: Synthetic peptide
HQGNTLPLT

SEQ ID NO: 396 - protein, 116 aa, Artificial Sequence
  Description of Artificial Sequence: Synthetic polypeptide
QMQLVQSGPEVKKPGTSVKVSCKASGYAFTSYNMYWVRQARGQRLEWIGYIDPYNADTTYNQKFKGRVTITRDMSTSTAYMELSSLRSEDTAVYYCLTTAYAMDYWGQGTLVTVSS

SEQ ID NO: 397 - protein, 106 aa, Artificial Sequence
  Description of Artificial Sequence: Synthetic polypeptide
DIQLTQSPSFLSASVGDRVTITCSASSTVNYMHWYQQKPGKAPKLLIYSTSNLASGVPSRFSGSGSGTEFTLTISSLQPEDFATYYCHQWSSYPYTFGQGTKLEIK

SEQ ID NO: 398 - protein, 10 aa, Artificial Sequence
  Description of Artificial Sequence: Synthetic peptide
GYAFTSYNMY

SEQ ID NO: 399 - protein, 17 aa, Artificial Sequence
  Description of Artificial Sequence: Synthetic peptide
YIDPYNADTTYNQKFKG

SEQ ID NO: 400 - protein, 7 aa, Artificial Sequence
  Description of Artificial Sequence: Synthetic peptide
TAYAMDY

SEQ ID NO: 401 - protein, 10 aa, Artificial Sequence
  Description of Artificial Sequence: Synthetic peptide
SASSTVNYMH

SEQ ID NO: 402 - protein, 7 aa, Artificial Sequence
  Description of Artificial Sequence: Synthetic peptide
STSNLAS

SEQ ID NO: 403 - protein, 9 aa, Artificial Sequence
  Description of Artificial Sequence: Synthetic peptide
HQWSSYPYT

SEQ ID NO: 404 - protein, 117 aa, Artificial Sequence
  Description of Artificial Sequence: Synthetic polypeptide
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 405 - protein, 5 aa, Artificial Sequence
  Description of Artificial Sequence: Synthetic peptide
INRMG

SEQ ID NO: 406 - protein, 16 aa, Artificial Sequence
  Description of Artificial Sequence: Synthetic peptide
SITVRGITNYADSVKG

SEQ ID NO: 407 - protein, 9 aa, Artificial Sequence
  Description of Artificial Sequence: Synthetic peptide
VSSNRDPDY

SEQ ID NO: 408 - protein, 9 aa, Artificial Sequence
  Description of Artificial Sequence: Synthetic peptide
  MOD_RES (2)..(2): Thr or Val
  MOD_RES (4)..(4): Ser, His, Gly or Pro
  MOD_RES (9)..(9): Ser, Thr or Gly
FXFXSFGMX

SEQ ID NO: 409 - protein, 17 aa, Artificial Sequence
  Description of Artificial Sequence: Synthetic peptide
  MOD_RES (5)..(5): Ser, Thr or Gly
  MOD_RES (7)..(7): Ser, Ala or Gly
  MOD_RES (9)..(9): Thr or Ala
  MOD_RES (11)..(11): Tyr or Thr
  MOD_RES (13)..(13): Asp or Pro
  MOD_RES (15)..(15): Val or Ala
  MOD_RES (16)..(16): Lys, Arg or Glu
SISGXGXDXLXAXSXXG

SEQ ID NO: 410 - protein, 8 aa, Artificial Sequence
  Description of Artificial Sequence: Synthetic peptide
  MOD_RES (1)..(1): Thr or Ser
  MOD_RES (2)..(2): Ile or Phe
  MOD_RES (3)..(3): Gly or Ala
  MOD_RES (6)..(6): Leu, Arg or Val
  MOD_RES (7)..(7): Ser, Ile, Arg, Lys, Val or Thr
  MOD_RES (8)..(8): Arg, Pro, Ile, Ala, Gln, Lys or Tyr
XXXGSXXX

SEQ ID NO: 411 - protein, 6 aa, Artificial Sequence
  Description of Artificial Sequence: Synthetic peptide
  MOD_RES (1)..(1): Gly or Ala
  MOD_RES (4)..(4): Leu, Arg or Val
  MOD_RES (5)..(5): Ser, Ile, Arg, Lys, Val or Thr
  MOD_RES (6)..(6): Arg, Pro, Ile, Ala, Gln, Lys or Tyr
XGSXXX

SEQ ID NO: 412 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 413 - protein, 112 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 414 - protein, 9 aa, Artificial Sequence
  Description of Artificial Sequence: Synthetic peptide
FNIKDYYMH

SEQ ID NO: 415 - protein, 5 aa, Artificial Sequence
  Description of Artificial Sequence: Synthetic peptide
DYYMH

SEQ ID NO: 416 - protein, 17 aa, Artificial Sequence
  Description of Artificial Sequence: Synthetic peptide
WIDLENANTIYDAKFQG

SEQ ID NO: 417 - protein, 12 aa, Artificial Sequence
  Description of Artificial Sequence: Synthetic peptide
ARDAYGRYFYDV

SEQ ID NO: 418 - protein, 10 aa, Artificial Sequence
  Description of Artificial Sequence: Synthetic peptide
DAYGRYFYDV

SEQ ID NO: 419 - protein, 17 aa, Artificial Sequence
  Description of Artificial Sequence: Synthetic peptide
KSSQSLLNARTGKNYLA

SEQ ID NO: 420 - protein, 7 aa, Artificial Sequence
  Description of Artificial Sequence: Synthetic peptide
WASTRES

SEQ ID NO: 421 - protein, 8 aa, Artificial Sequence
  Description of Artificial Sequence: Synthetic peptide
KQSYSRRT

SEQ ID NO: 422 - protein, 251 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 423 - protein, 251 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGCGTKVEIKGGGGSGGGGSGGGGSGGGGSQVQLVQSGAEVKKPGASVKVSCKASGFNIKDYYMHWVRQAPGQCLEWMGWIDLENANTIYDAKFQGRVTITRDTSASTAYMELSSLRSEDTAVYYCARDAYGRYFYDVWGQGTLVTVSS

SEQ ID NO: 424 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDQYGRYFYDVWGQGTLVTVSS

SEQ ID NO: 425 - protein, 12 aa, Artificial Sequence
  Description of Artificial Sequence: Synthetic peptide
ARDQYGRYFYDV

SEQ ID NO: 426 - protein, 10 aa, Artificial Sequence
  Description of Artificial Sequence: Synthetic peptide
DQYGRYFYDV

SEQ ID NO: 427 - protein, 251 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDQYGRYFYDVWGQGTLVTVSS

SEQ ID NO: 428 - protein, 251 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGCGTKVEIKGGGGSGGGGSGGGGSGGGGSQVQLVQSGAEVKKPGASVKVSCKASGFNIKDYYMHWVRQAPGQCLEWMGWIDLENANTIYDAKFQGRVTITRDTSASTAYMELSSLRSEDTAVYYCARDQYGRYFYDVWGQGTLVTVSS

SEQ ID NO: 429 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSS

SEQ ID NO: 430 - protein, 112 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSLRTFGGGTKVEIK

SEQ ID NO: 431 - protein, 17 aa, Artificial Sequence
  Description of Artificial Sequence: Synthetic peptide
WIDLEEGNTIYDAKFQG

SEQ ID NO: 432 - protein, 8 aa, Artificial Sequence
  Description of Artificial Sequence: Synthetic peptide
KQSYSLRT

SEQ ID NO: 433 - protein, 251 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSLRTFGGGTKVEIKGGGGSGGGGSGGGGSGGGGSQVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSS

SEQ ID NO: 434 - protein, 251 aa, Artificial Sequence
  Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSLRTFGCGTKVEIKGGGGSGGGGSGGGGSGGGGSQVQLVQSGAEVKKPGASVKVSCKASGFNIKDYYMHWVRQAPGQCLEWMGWIDLEEGNTIYDAKFQGRVTITRDTSASTAYMELSSLRSEDTAVYYCARDAYGRYFYDVWGQGTLVTVSS

SEQ ID NO: 435 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDVYGRYFYDLWGQGTLVTVSS

SEQ ID NO: 436 - protein, 112 aa, Artificial Sequence
  Description of Artificial Sequence: Synthetic polypeptide
DIVMTQSPDSLAVSLGERATINCKSSQSLLESRTGKNYLAWYQQKPGQPPKLLIYWASTRESGVPDRFSGSGSGTDFTLTISSLQAEDVAVYYCKQSYSRRTFGGGTKVEIK

SEQ ID NO: 437 - protein, 9 aa, Artificial Sequence
  Description of Artificial Sequence: Synthetic peptide
FNIKDYYMH

SEQ ID NO: 438 - protein, 17 aa, Artificial Sequence
  Description of Artificial Sequence: Synthetic peptide
WIDLENANTIYDAKFQG

SEQ ID NO: 439 - protein, 12 aa, Artificial Sequence
  Description of Artificial Sequence: Synthetic peptide
ARDVYGRYFYDL

SEQ ID NO: 440 - protein, 17 aa, Artificial Sequence
  Description of Artificial Sequence: Synthetic peptide
KSSQSLLNARTGKNYLA

SEQ ID NO: 441 - protein, 7 aa, Artificial Sequence
  Description of Artificial Sequence: Synthetic peptide
WASTRES

SEQ ID NO: 442 - protein, 8 aa, Artificial Sequence
  Description of Artificial Sequence: Synthetic peptide
KQSYSRRT

SEQ ID NO: 443 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGGYFYDVWGQGTLVTVSS

SEQ ID NO: 444 - protein, 112 aa, Artificial Sequence
  Description of Artificial Sequence: Synthetic polypeptide
DIVMTQSPDSLAVSLGERATINCKSSQSLLNSRTGKNYLAWYQQKPGQPPKLLIYWASTRESGVPDRFSGSGSGTDFTLTISSLQAEDVAVYYCKQSYSRRTFGCGTKVEIK

SEQ ID NO: 445 - protein, 9 aa, Artificial Sequence
  Description of Artificial Sequence: Synthetic peptide
FNIKDYYMH

SEQ ID NO: 446 - protein, 17 aa, Artificial Sequence
  Description of Artificial Sequence: Synthetic peptide
WIDLENANTIYDAKFQG

SEQ ID NO: 447 - protein, 12 aa, Artificial Sequence
  Description of Artificial Sequence: Synthetic peptide
ARDAYGGYFYDV

SEQ ID NO: 448 - protein, 17 aa, Artificial Sequence
  Description of Artificial Sequence: Synthetic peptide
KSSQSLLNARTGKNYLA

SEQ ID NO: 449 - protein, 7 aa, Artificial Sequence
  Description of Artificial Sequence: Synthetic peptide
WASTRES

SEQ ID NO: 450 - protein, 8 aa, Artificial Sequence
  Description of Artificial Sequence: Synthetic peptide
KQSYSRRT

SEQ ID NO: 451 - protein, 125 aa, Artificial Sequence
  Description of Artificial Sequence: Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFGNYAVSWFAHWGQGTLVTVSS

SEQ ID NO: 452 - protein, 109 aa, Artificial Sequence
  Description of Artificial Sequence: Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 453 - protein, 9 aa, Artificial Sequence
  Description of Artificial Sequence: Synthetic peptide
FTFDTYAMN

SEQ ID NO: 454 - protein, 19 aa, Artificial Sequence
  Description of Artificial Sequence: Synthetic peptide
RIRSKYNNYATYYADSVKD

SEQ ID NO: 455 - protein, 16 aa, Artificial Sequence
  Description of Artificial Sequence: Synthetic peptide
VRHGNFGNYAVSWFAH

SEQ ID NO: 456 - protein, 14 aa, Artificial Sequence
  Description of Artificial Sequence: Synthetic peptide
GSSTGAVTTSNYAN

SEQ ID NO: 457 - protein, 7 aa, Artificial Sequence
  Description of Artificial Sequence: Synthetic peptide
GTDKRAP

SEQ ID NO: 458 - protein, 9 aa, Artificial Sequence
  Description of Artificial Sequence: Synthetic peptide
ALWYSNHWV

SEQ ID NO: 459 - protein, 125 aa, Artificial Sequence
  Description of Artificial Sequence: Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGSFGNHIVSWFAHWGQGTLVTVSS

SEQ ID NO: 460 - protein, 109 aa, Artificial Sequence; identical to SEQ ID NO: 452
  Description of Artificial Sequence: Synthetic polypeptide

SEQ ID NO: 461 - protein, 9 aa, Artificial Sequence
  Description of Artificial Sequence: Synthetic peptide
FTFDTYAMN

SEQ ID NO: 462 - protein, 19 aa, Artificial Sequence
  Description of Artificial Sequence: Synthetic peptide
RIRSKYNNYATYYADSVKD

SEQ ID NO: 463 - protein, 16 aa, Artificial Sequence
  Description of Artificial Sequence: Synthetic peptide
VRHGSFGNHIVSWFAH

SEQ ID NO: 464 - protein, 14 aa, Artificial Sequence
  Description of Artificial Sequence: Synthetic peptide
GSSTGAVTTSNYAN

SEQ ID NO: 465 - protein, 7 aa, Artificial Sequence
  Description of Artificial Sequence: Synthetic peptide
GTDKRAP

SEQ ID NO: 466 - protein, 9 aa, Artificial Sequence
  Description of Artificial Sequence: Synthetic peptide
ALWYSNHWV

SEQ ID NO: 467 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYDAKFQGRVTITRDTSASTAYMELSSLRSEDTAVYYCARDGYGRYFYDVWGQGTLVTVSS

SEQ ID NO: 468 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYSRRTFGGGTKVEIK

SEQ ID NO: 469 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDVYGRYLYDVWGQGTLVTVSS

SEQ ID NO: 470 - protein, 112 aa, Artificial Sequence
  Description of Artificial Sequence: Synthetic polypeptide
DIVMTQSPDSLAVSLGERATINCKSSQSLLNNRTRKNYLAWYQQKPGQPPKLLIYWASTRESGVPDRFSGSGSGTDFTLTISSLQAEDVAVYYCKQSYSRRTFGGGTKVEIK

SEQ ID NO: 471 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDAYGRYFYDVWGQGTLVTVSS

SEQ ID NO: 472 - protein, 112 aa, Artificial Sequence
  Description of Artificial Sequence: Synthetic polypeptide
DIVMTQSPDSLAVSLGERATINCRSSQSLLNSRTRKNYLAWYQQKPGQPPKLLIYWASTRESGVPDRFSGSGSGTDFTLTISSLQAEDVAVYYCKQSYSRRTFGGGTKVEIK

SEQ ID NO: 473 - protein, 112 aa, Artificial Sequence
  Description of Artificial Sequence: Synthetic polypeptide
DIVMTQSPDSLAVSLGERATINCKSSQSLLNGRTRKNYLAWYQQKPGQPPKLLIYWASTRESGVPDRFSGSGTGTDFTLTISSLQAEDVAVYYCKQSYSRRTFGGGTKVEIK

SEQ ID NO: 474 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDNYGGYFYDVWGQGTLVTVSS

SEQ ID NO: 475 - protein, 112 aa, Artificial Sequence
  Description of Artificial Sequence: Synthetic polypeptide
DIVMTQSPDSLAVPLGERATINCKSSQSLLNSRTRKNYLAWYQQKPGQPPKLLIYWASTRESGVPDRFSGSGSGTDFTLTISSLQAEDVAVYYCKQSYSRRTFGGGTKVEIK

SEQ ID NO: 476 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYFYDVWGQGTLVTVSS

SEQ ID NO: 477 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYSLRTFGGGTKVEIK

SEQ ID NO: 478 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYFFDVWGQGTLVTVSS

SEQ ID NO: 479 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLRTFGGGTKVEIK

SEQ ID NO: 480 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCAREGYGRYFYDVWGQGTLVTVSS

SEQ ID NO: 481 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYFRRAFGGGTKVEIK

SEQ ID NO: 482 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYYYDVWGQGTLVTVSS

SEQ ID NO: 483 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLRTFGGGTKLEIK

SEQ ID NO: 484 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYQPKFQGRVTITRDTSASTAYMELSSLRSEDTAVYYCARDGYGRYFYDVWGQGTLVTVSS

SEQ ID NO: 485 - protein, 112 aa, Artificial Sequence
  Description of Artificial Sequence: Synthetic polypeptide
DIVMTQSPDSLAVSLGERATINCKSSQSLLNSRTRKNYLAWYQQKPGQSPKLLIYWASTRESGVPDRFTGSGSGTDFTLTISSLQAEDVAVYYCKQSYSLRTFGGGTKVEIK

SEQ ID NO: 486 - protein, 119 aa, Artificial Sequence
  Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYFYDYWGQGTLVTVSS

SEQ ID NO: 487 - protein, 112 aa, Artificial Sequence
  Description of Artificial Sequence: Synthetic polypeptide
DIVMTQSPDSLAVSLGERATINCKSSQSLLESRTGKNYLAWYQQKPGQPPKLLIYWASTRESGVPDRFSGSGSGTDFTLTISSLQAEDVAVYYCKQSYSLRTFGGGTKVEIK

SEQ ID NO: 488 - protein, 121 aa, Artificial Sequence
  Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 489 - protein, 108 aa, Artificial Sequence
  Description of Artificial Sequence: Synthetic polypeptide
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 490 - protein, 243 aa, Artificial Sequence
  Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 491 - protein, 118 aa, Artificial Sequence [sequence omitted]

SEQ ID NO: 492 - protein, 108 aa, Artificial Sequence [sequence omitted]

SEQ ID NO: 493 - protein, 5 aa, Artificial Sequence
  Description of Artificial Sequence: Synthetic peptide
GYGMH

SEQ ID NO: 494 - protein, 17 aa, Artificial Sequence
  Description of Artificial Sequence: Synthetic peptide
VIWYDGSKKYYVDSVKG

SEQ ID NO: 495 - protein, 9 aa, Artificial Sequence
  Description of Artificial Sequence: Synthetic peptide
QMGYWHFDL

SEQ ID NO: 496 - protein, 11 aa, Artificial Sequence
  Description of Artificial Sequence: Synthetic peptide
RASQSVSSYLA

SEQ ID NO: 497 - protein, 7 aa, Artificial Sequence
  Description of Artificial Sequence: Synthetic peptide
DASNRAT

SEQ ID NO: 498 - protein, 10 aa, Artificial Sequence
  Description of Artificial Sequence: Synthetic peptide
QQRSNWPPLT

SEQ ID NO: 499 - protein, 119 aa, Artificial Sequence [sequence omitted]

SEQ ID NO: 500 - protein, 101 aa, Artificial Sequence
  Description of Artificial Sequence: Synthetic polypeptide
DFMLTQPHSVSESPGKTVIISCWYQQRPGRAPTTVIFGVPDRFSGSIDRSSNSASLTISGLQTEDEADYYCFGGGTKLTVLGQPKAAPSVTLFPPSSEELQ

SEQ ID NO: 501 - protein, 119 aa, Artificial Sequence [sequence omitted]

SEQ ID NO: 502 - protein, 111 aa, Artificial Sequence
  Description of Artificial Sequence: Synthetic polypeptide
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 503 - protein, 119 aa, Artificial Sequence
  Description of Artificial Sequence: Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 504 - protein, 106 aa, Artificial Sequence
  Description of Artificial Sequence: Synthetic polypeptide
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 505 - protein, 243 aa, Artificial Sequence
  Description of Artificial Sequence: Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 506 - protein, 119 aa, Artificial Sequence [sequence omitted]

SEQ ID NO: 507 - protein, 107 aa, Artificial Sequence
  Description of Artificial Sequence: Synthetic polypeptide
DDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 508 - protein, 119 aa, Artificial Sequence
  Description of Artificial Sequence: Synthetic polypeptide
QVQLVQSGGGVVQPGRSLRLSCKASGYTFTRYTMHWVRQAPGKGLEWIGYINPSRGYTNYNQKFKDRFTISTDKSKSTAFLQMDSLRPEDTAVYYCARYYDDHYCLDYWGQGTPVTVSS

SEQ ID NO: 509 - protein, 107 aa, Artificial Sequence
  Description of Artificial Sequence: Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 510-599 - skipped (90 entries)

SEQ ID NO: 600 - protein, 125 aa, Artificial Sequence
  Description of Artificial Sequence: Synthetic polypeptide
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 601 - protein, 109 aa, Artificial Sequence
  Description of Artificial Sequence: Synthetic polypeptide
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 602 - protein, 123 aa, Artificial Sequence
  Description of Artificial Sequence: Synthetic polypeptide
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 603 - protein, 108 aa, Artificial Sequence
  Description of Artificial Sequence: Synthetic polypeptide
AEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 604 - protein, 119 aa, Artificial Sequence
  Description of Artificial Sequence: Synthetic polypeptide
QVQLVQSGAEVKKPGSSVKVSCKASGYSFTTYYIHWVRQAPGQGLEWMGWIFPGNDNIKYSEKFKGRVTITADKSTSTAYMELSSLRSEDTAVYYCAIDSVSIYYFDYWGQGTLVTVSS

SEQ ID NO: 605 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRKSGVPDRFSGSGSGTDFTLTISSLQAEDVAVYYCTQSFILRTFGGGTKVEIK

SEQ ID NO: 606 - protein, 10 aa, Artificial Sequence
  Description of Artificial Sequence: Synthetic peptide
GYSFTTYYIH

SEQ ID NO: 607 - protein, 17 aa, Artificial Sequence
  Description of Artificial Sequence: Synthetic peptide
WIFPGNDNIKYSEKFKG

SEQ ID NO: 608 - protein, 10 aa, Artificial Sequence
  Description of Artificial Sequence: Synthetic peptide
DSVSIYYFDY

SEQ ID NO: 609 - protein, 17 aa, Artificial Sequence
  Description of Artificial Sequence: Synthetic peptide
KSSQSLLNSRTRKNYLA

SEQ ID NO: 610 - protein, 7 aa, Artificial Sequence
  Description of Artificial Sequence: Synthetic peptide
WASTRKS

SEQ ID NO: 611 - protein, 8 aa, Artificial Sequence
  Description of Artificial Sequence: Synthetic peptide
TQSFILRT

SEQ ID NO: 612 - protein, 119 aa, Artificial Sequence
  Description of Artificial Sequence: Synthetic polypeptide
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSS

SEQ ID NO: 613 - protein, 112 aa, Artificial Sequence
  Description of Artificial Sequence: Synthetic polypeptide
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIK

SEQ ID NO: 614 - protein, 10 aa, Artificial Sequence
  Description of Artificial Sequence: Synthetic peptide
GFAFTDYYIH

SEQ ID NO: 615 - protein, 17 aa, Artificial Sequence
  Description of Artificial Sequence: Synthetic peptide
WISPGNVNTKYNENFKG

SEQ ID NO: 616 - protein, 10 aa, Artificial Sequence
  Description of Artificial Sequence: Synthetic peptide
DGYSLYYFDY

SEQ ID NO: 617 - protein, 17 aa, Artificial Sequence
  Description of Artificial Sequence: Synthetic peptide
KSSQSLLNSRTRKNYLA

SEQ ID NO: 618 - protein, 7 aa, Artificial Sequence
  Description of Artificial Sequence: Synthetic peptide
WASTRQS

SEQ ID NO: 619 - protein, 8 aa, Artificial Sequence
  Description of Artificial Sequence: Synthetic peptide
TQSHTLRT

SEQ ID NO: 620 - protein, 120 aa, Artificial Sequence
  Description of Artificial Sequence: Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYNYWGQGTQVTV

SEQ ID NO: 621 - protein, 120 aa, Artificial Sequence
  Description of Artificial Sequence: Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYDYWGQGTQVTV

SEQ ID NO: 622 - protein, 120 aa, Artificial Sequence
  Description of Artificial Sequence: Synthetic polypeptide
MAESGGGSVQTGGSLRLSCAYTASSVCMAWFRQAPGKEREGVAVTREGLTQTGYADSVKGRFAISQDYAKKTLYLQMSSLKPEDTARYYCAARPTSPCTVDGELLASTYNYWGQGTQVTV

SEQ ID NO: 623 - protein, 120 aa, Artificial Sequence
  Description of Artificial Sequence: Synthetic polypeptide
MAESGGGSVQTGGSLRLSCAYTASSVCMAWFRQAPGKEREGVAVTREGLTQTGYADSVKGRFAISQDYAKKTLYLQMSSLKPEDTARYYCAARPTSPCTVDGELLASTYDYWGQGTQVTV

SEQ ID NO: 624-710 - skipped (87 entries)

SEQ ID NO: 711 - protein, 10 aa, Artificial Sequence
  Description of Artificial Sequence: Synthetic peptide
HHHHHHHHHH

SEQ ID NO: 712 - protein, 7 aa, Artificial Sequence
  Description of Artificial Sequence: Synthetic peptide
DASSLES

SEQ ID NO: 713 - protein, 40 aa, Artificial Sequence
  Description of Artificial Sequence: Synthetic polypeptide
  MISC_FEATURE (1)..(40): This sequence may encompass 1-20 “Gly Ser” repeating units
GSGSGSGSGSGSGSGSGSGSGSGSGSGSGSGSGSGSGSGS

SEQ ID NO: 714 - protein, 60 aa, Artificial Sequence
  Description of Artificial Sequence: Synthetic polypeptide
  MISC_FEATURE (1)..(60): This sequence may encompass 1-20 “Gly Gly Ser” repeating units
GGSGGSGGSGGSGGSGGSGGSGGSGGSGGSGGSGGSGGSGGSGGSGGSGGSGGSGGSGGS

SEQ ID NO: 715 - protein, 80 aa, Artificial Sequence
  Description of Artificial Sequence: Synthetic polypeptide
  MISC_FEATURE (1)..(80): This sequence may encompass 1-20 “Gly Gly Gly Ser” repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 716 - protein, 80 aa, Artificial Sequence
  Description of Artificial Sequence: Synthetic polypeptide
  MISC_FEATURE (1)..(80): This sequence may encompass 1-20 “Gly Gly Ser Gly” repeating units
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 717 - protein, 100 aa, Artificial Sequence; identical to SEQ ID NO: 301
  Description of Artificial Sequence: Synthetic polypeptide
  MISC_FEATURE (1)..(100): This sequence may encompass 1-20 “Gly Gly Ser Gly Gly” repeating units

SEQ ID NO: 718 - protein, 100 aa, Artificial Sequence; identical to SEQ ID NO: 300
  Description of Artificial Sequence: Synthetic polypeptide
  MISC_FEATURE (1)..(100): This sequence may encompass 1-20 “Gly Gly Gly Gly Ser” repeating units

SEQ ID NO: 719 - protein, 6 aa, Artificial Sequence
  Description of Artificial Sequence: Synthetic 6xHis tag
HHHHHH